(19) United States
(12) Patent Application Publication
(10) Pub. No.: US 2024/0287010 A1
(43) Pub. Date: Aug. 29, 2024

(54) Process For Making Epoxide Derivatives of Farnesene and Use Thereof in Further Synthesis

(71) Applicant: Givaudan SA, VERNIER (CH)

(72) Inventors: Giacomo PERSIANI, Basel (CH); Corinne BAUMGARTNER, Hinwil (CH); Thierry GRANIER, Duebendorf (CH); Martin LOVCHIK, Duebendorf (CH); Fridtjof SCHROEDER, Hettlingen (CH)

(21) Appl. No.: 18/566,075

(22) PCT Filed: Jun. 21, 2022
(86) PCT No.: PCT/EP2022/066928

§ 371 Date: Nov. 30, 2023

(30) Foreign Application Priority Data
Jun. 23, 2021 (GB) 2108985.9

(51) Int. Cl. C07D 303/04; C07C 33/02; C07C 69/732

(52) U.S. Cl. CPC C07D 303/04; C07C 33/02; C07C 69/732

(57) ABSTRACT

Described herein are methods for making intermediates in the production of fragrance ingredients starting from β-farnesene. In particular, methods for making acetylmethylfarnesol and hydroxy-farnesylacetone are described. Methods for making other intermediates in the process are also described. In addition, compositions comprising intermediates in the process and uses of the intermediates are described.

### TECHNICAL FIELD

The present invention relates to methods for making intermediates in the production of fragrances such as Amberketal and Amberketal homologues starting β-farnesene. In particular, the invention relates to the production of acetylmethylfarnesol and hydroxy-farnesylacetone.

### BACKGROUND

Amberketal provides a powerful and tenacious ambery and woody odour that is useful in fragrance compositions alone or in combination with other woody or ambery ingredients. Amberketal is traditionally prepared from Manool via a number of chemical transformations. However, the supply of natural Manool is limited. It is therefore desirable to provide a new efficient and cost effective synthetic route to obtain Amberketal and Amberketal homologues.

A synthetic route to Amberketal homologues (Formula A) comprising contacting a compound of Formula B (wherein R¹ is H, methyl or ethyl) with a squalene-hopene cyclase (SHC) enzyme or enzyme variant has been disclosed in WO2021/209482 (PCT/EP2021/059618).

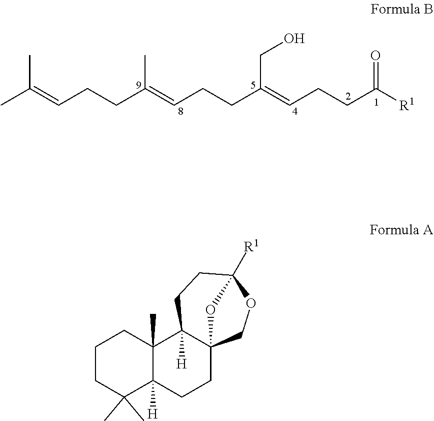

It is therefore desirable to provide a new efficient access to obtain compounds of Formula B, which can then be used in the production of Amberketal and Amberketal homologues.

### SUMMARY

In accordance with a first aspect of the present invention, there is provided a method for making a compound of Formula (VIII), a compound of Formula (IX) or a mixture thereof,

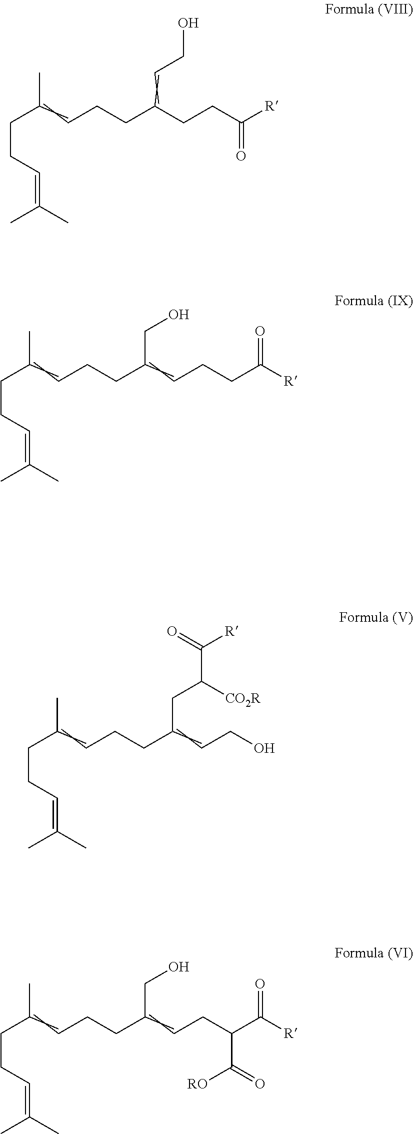

wherein the method comprises hydrolysis and decarboxylation of a compound of Formula (V), a compound of Formula (VI), or a mixture thereof, respectively, wherein R′ is selected from the group consisting of C1 to C6 alkyl (e.g. methyl, ethyl, propyl, isopropyl) and an OR group, and wherein R is selected from the group consisting of hydrogen and a C1 to C6 alkyl group (e.g. methyl, ethyl, propyl, isopropyl), for example, by contacting the compound of Formula (V), the compound of Formula (VI), or the mixture thereof with an acid, a base, water (e.g., water in a polar solvent such as DMSO), or a nucleophile (e.g., a salt, such as a halide salt, a cyanide salt or a thiolate, in a polar solvent).

In a second aspect of the present invention, there is provided a method for making a compound of Formula (VIII), a compound of Formula (IX) or a mixture thereof,

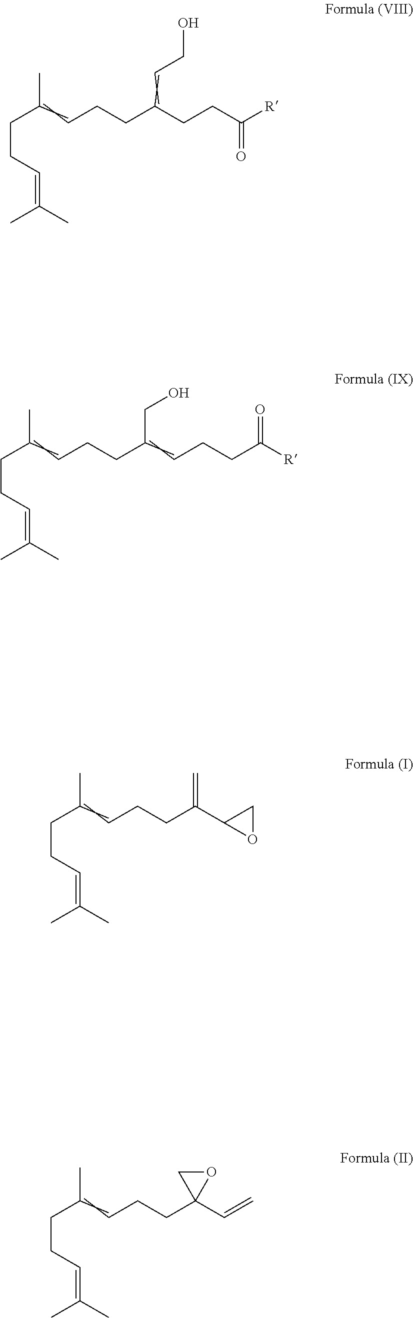

wherein R′ is methyl and wherein the method comprises contacting a compound of Formula (I), a compound of Formula (II) or a mixture thereof, respectively, with acetone and a palladium catalyst.

In a third aspect of the present invention, there is provided a method for making a compound of Formula (IX), a compound of Formula (VIII), or a mixture thereof, wherein R′ is methyl,

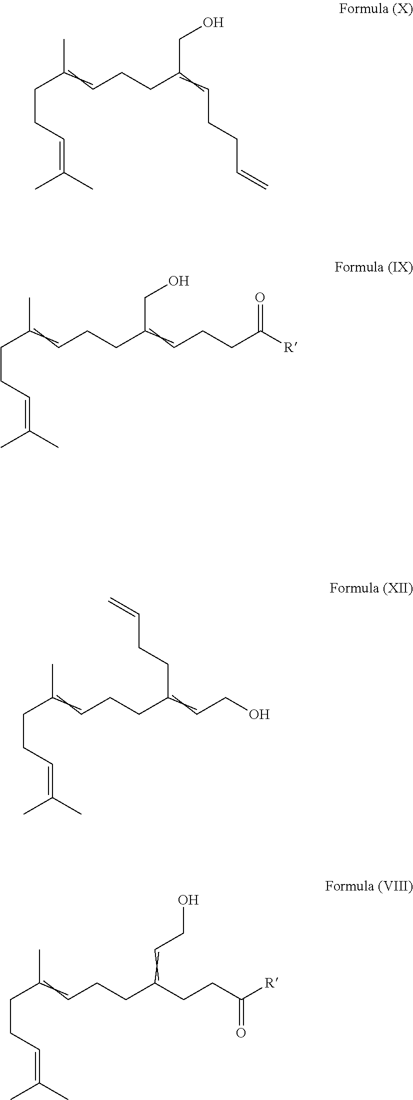

the method comprising a palladium-catalysed Wacker oxidation of a compound of Formula (X), a compound of Formula (XII), or a mixture thereof, such as contacting the compound of Formula (X), a compound of Formula (XII), or a mixture thereof, respectively, with a palladium catalyst (e.g., palladium (II) chloride) and gaseous oxygen.

In accordance with a fourth aspect of the present invention, there is provided a method for making a compound of Formula (V), a compound of Formula (VI) or a mixture thereof,

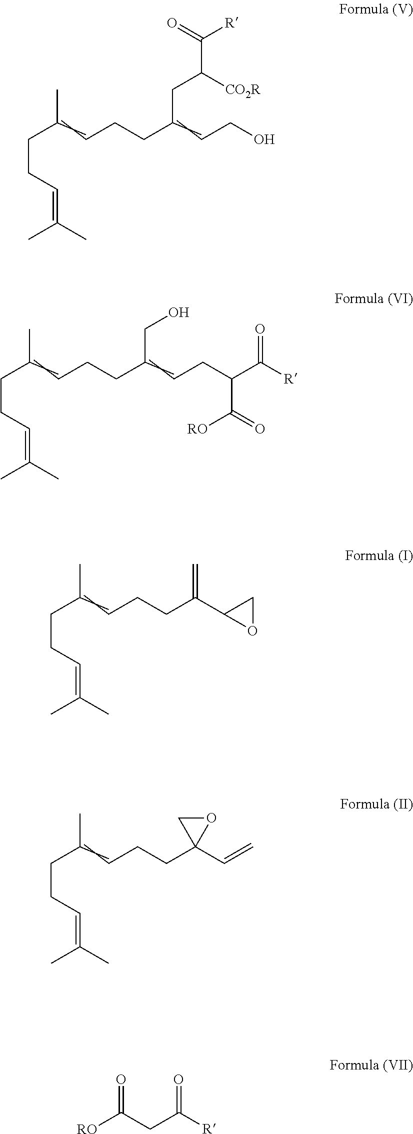

wherein the method comprises contacting a compound of Formula (I), a compound of Formula (II) or a mixture thereof, respectively, with a compound of Formula (VII) and a palladium catalyst, wherein R′ is selected from the group consisting of a C1 to C6 alkyl and an OR group, and R is selected from the group consisting of hydrogen and a C1 to C6 alkyl group.

In a fifth aspect of the present invention, there is provided a method for making a compound of Formula (X), a compound of Formula (XII), or a mixture thereof,

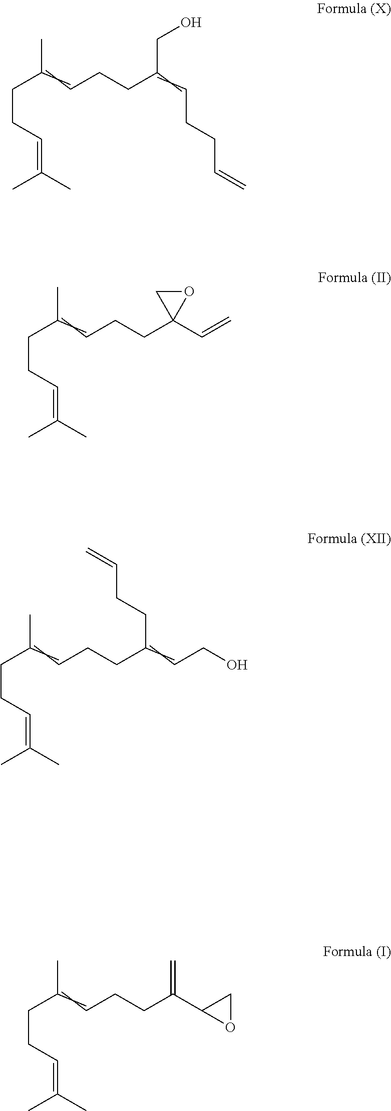

wherein the method comprises a copper-catalysed alkylation of a compound of Formula (II), a compound of Formula (I), or a mixture thereof, such as contacting the compound of Formula (II), a compound of Formula (I), or a mixture thereof with a copper catalyst and a Grignard reagent, such as an allyl magnesium halide.

In accordance with a sixth aspect of the present invention, there is provided a method for making a compound of Formula (I), a compound of Formula (II) or a mixture thereof,

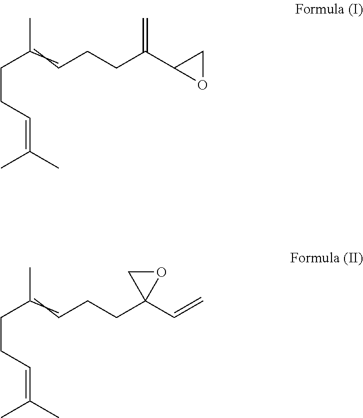 [SMILES: C=C(CCC=C(C)CCC=C(C)C)C1CO1 ; C=CC1(CCC=C(C)CCC=C(C)C)CO1]

wherein the method comprises contacting a compound of Formula (III) with a catalyst comprising a metal complexed with a salen ligand or a porphyrin ligand, wherein the salen ligand has Formula (IV) and wherein the porphyrin ligand has Formula (XI)

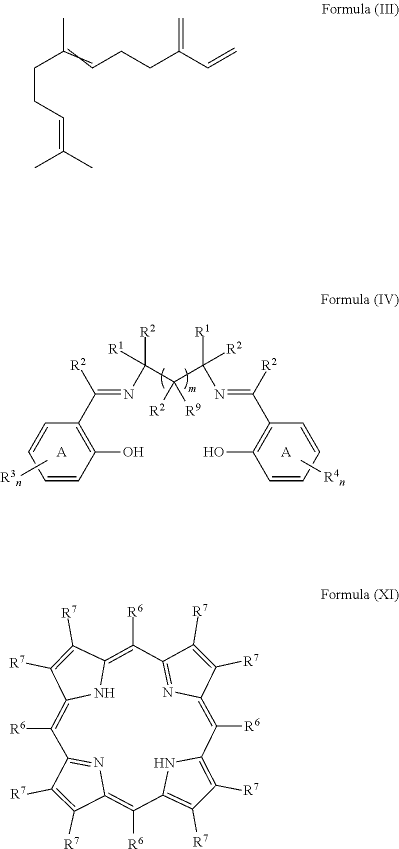

wherein
each ring A is independently selected from phenyl or naphthyl; each R¹ is independently hydrogen, an alkyl group, an aryl group, or both R¹ together with the carbon atoms to which R¹ are attached form a cycloalkylene group, an arylene group, two or more cycloalkylene groups connected by a single bond or an alkyl group or two or more arylene groups connected by a single bond or an alkyl group, or a cycloalkylene and an arylene group connected by a single bond or an alkyl group;
each R² is independently hydrogen or an alkyl group;
R³ is independently an alkyl group, an alkoxy group, an alkylaryl group, an alkylsilyl ether group, or a halide;
R⁴ is independently an alkyl group, an alkoxy group, an alkylaryl group, an alkylsilyl ether group, or a halide;
R⁹ is selected from hydrogen, an optionally substituted alkyl group, an optionally substituted aryl group, a hydroxyl, an amino group;
each n is selected from 0 to 4;
m is selected from 0 to 4;

each R⁶ is independently selected from hydrogen, an optionally substituted alkyl group, an optionally substituted aryl group, or an optionally substituted heteroaromatic group; and
each R⁷ is independently selected from hydrogen, an alkyl group, an ester-containing alkyl group or a carboxylic acid containing alkyl group.

In a seventh aspect of the present invention, there is provided a method of separation of a mixture of compounds of Formulae (I) and (II),

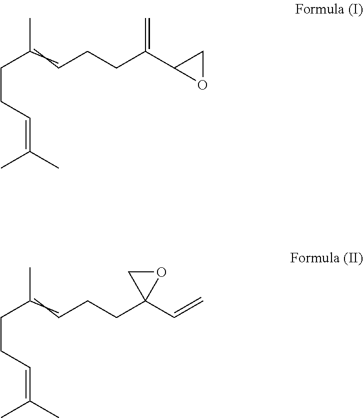 [SMILES: C=C(CCC=C(C)CCC=C(C)C)C1CO1 ; C=CC1(CCC=C(C)CCC=C(C)C)CO1]

the method comprising the distillation of the mixture of compounds of Formulae (I) and (II).

In an eight aspect of the present invention, there is provided a compound of Formula (V), a compound of Formula (VI) or a mixture thereof, wherein R′ is selected from the group consisting of a C1 to C6 alkyl and an OR group; and R is selected from the group consisting of hydrogen and C1 to C6 alkyl.

In a ninth aspect of the present invention, there is provided a compound of Formula (X).

In a tenth aspect of the present invention, there is provided a compound of Formula (VIII), a compound of Formula (IX) or a mixture thereof obtained by or obtainable by the method of the first aspect or the second aspect.

In an eleventh aspect of the present invention, there is provided a compound of Formula (IX) obtained by or obtainable by the method of the third aspect.

In a twelfth aspect of the present invention, there is provided a compound of Formula (V), a compound of Formula (VI) or a mixture thereof obtained by or obtainable by the method of the fourth aspect.

In a thirteenth aspect of the present invention, there is provided a compound of Formula (X) obtained by or obtainable by the method of the fifth aspect.

In a fourteenth aspect of the present invention, there is provided a compound of Formula (I), a compound of Formula (II) or a mixture thereof obtained by or obtainable by the method of the sixth aspect.

In a fifteenth aspect of the present invention, there is provided a composition comprising a compound of Formula (V), a compound of Formula (VI) or a mixture thereof, wherein R′ is selected from the group consisting of a C1 to C6 alkyl and an OR group; and R is selected from the group consisting of hydrogen and C1 to C6 alkyl.

In a sixteenth aspect of the present invention, there is provided a composition comprising a compound of Formula (X).

In a seventeenth aspect of the present invention, there is provided the use of a composition comprising a compound of Formula (V), a compound of Formula (VI) or a mixture thereof in a method for making a fragrance composition, wherein R′ is selected from the group consisting of a C1 to C6 alkyl and an OR group; and R is selected from the group consisting of hydrogen and C1 to C6 alkyl.

In a eighteenth aspect of the present invention, there is provided the use of a composition comprising a compound of Formula (X) in a method for making a fragrance composition. In a nineteenth aspect of the present invention, there is provided a method for making a fragrance composition comprising a method according to the first aspect, the second aspect, the third aspect, the fourth aspect, the fifth aspect, the sixth aspect or the seventh aspect.

The details, examples and preferences provided in relation to any particular one or more of the stated aspects of the present invention will be further described herein and apply equally to all aspects of the present invention. Any combination of the embodiments, examples and preferences described herein in all possible variations thereof is encompassed by the present invention unless otherwise indicated herein, or otherwise clearly contradicted by context.

### BRIEF DESCRIPTION OF THE FIGURES

FIG. 1 shows the reaction scheme for the production of a compound of Formula (VIII), a compound of Formula (IX) or a mixture thereof from a compound of Formula (III) via various intermediate compounds.

FIG. 2 shows Formulae (I) to (XII).

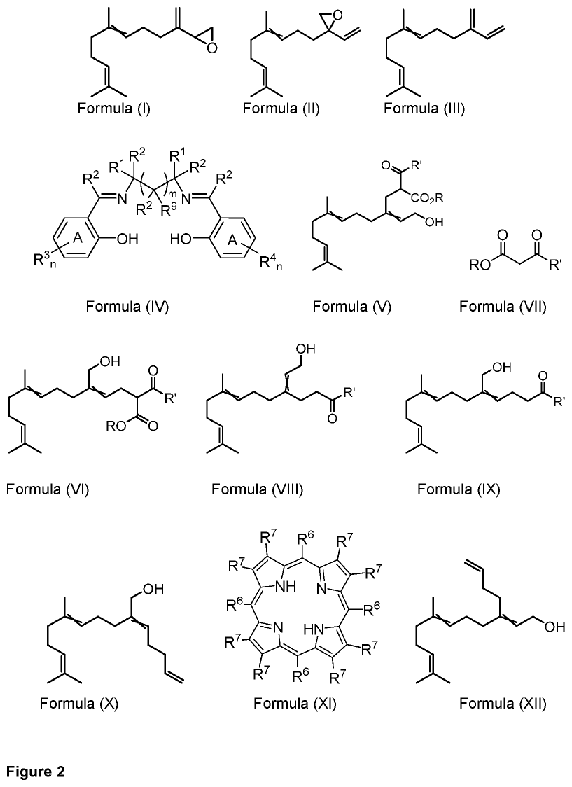

### DETAILED DESCRIPTION

The present invention provides novel and surprising methods for the production of a compound of Formula (VIII), a compound of Formula (IX) or mixtures thereof, which may be used in the production of Amberketal and Amberketal homologues.

In particular, the present invention is based, at least in part, on the surprising finding that a catalyst comprising a metal complexed with a salen ligand selectively oxidises a compound of Formula (III) at the 1,3-diene unit, producing a compound of Formula (I), a compound of Formula (II), or a mixture thereof. It is particularly surprising that neither of the other double bonds in the compound are oxidised, resulting in only two of the possible regioisomeric products, that is, compounds of Formula (I) and Formula (II). This surprising finding allows, for example, the selective production of compounds of Formulae (VIII) and (IX) and mixtures thereof.

Although the Jacobsen epoxidation (sometimes called the Jacobsen-Katsuki epoxidation) is known to allow enantioselective epoxidation of un-functionalized alkyl- and aryl-substituted alkenes and conjugated dienes, the compound of Formula (III) is a tetraene, which is a more challenging substrate due to the potential for epoxidation to occur at multiple different double bonds. However, surprisingly the inventors have found that the reaction selectively produces only compounds of Formula (I) and Formula (II), with no epoxidation occurring at the remaining two carbon-carbon double bonds, significantly reducing the number of undesired side products produced from the epoxidation reaction.

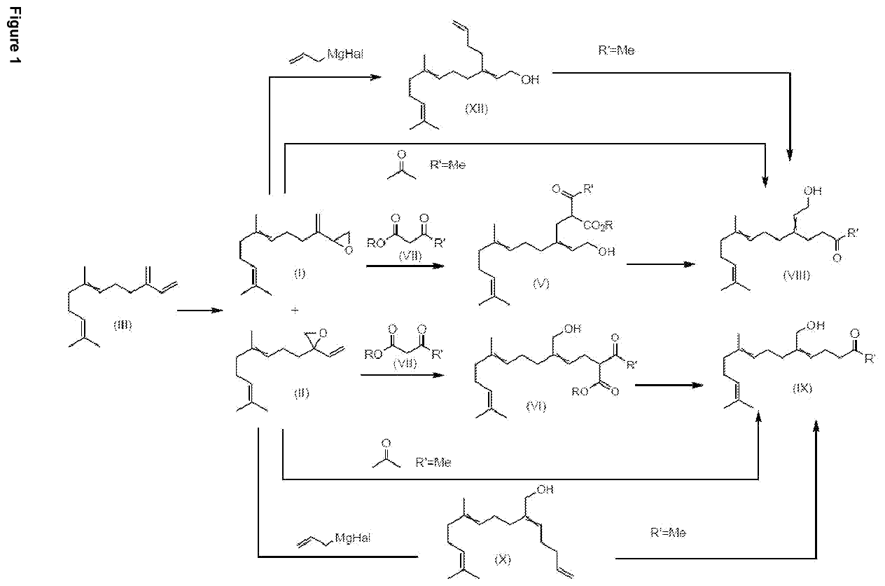

The synthetic routes of the present invention are summarised in the schematic shown in FIG. 1.

### Method for Making Compounds of Formulae (I) and (II)

There is provided herein in an aspect a method for making a compound of Formula (I), a compound of Formula (II) or a mixture thereof,

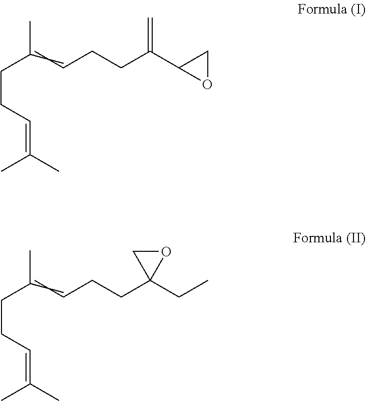

wherein the method comprises contacting a compound of Formula (III) with a catalyst comprising a metal complexed with a salen ligand or a porphyrin ligand, wherein the salen ligand has Formula (IV); and wherein the porphyrin ligand has Formula (XI),

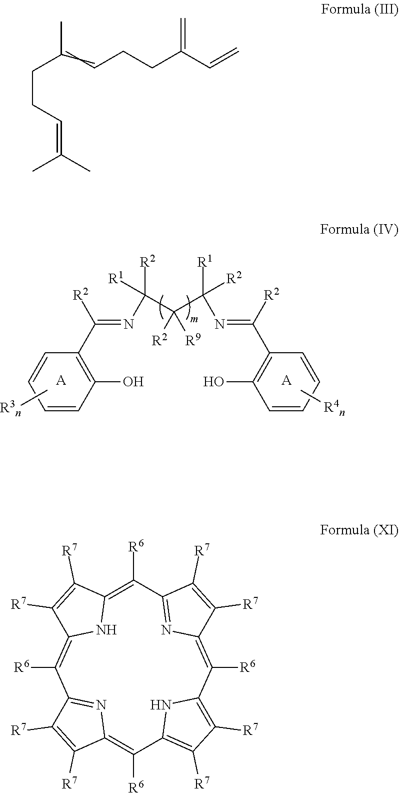

wherein each ring A is independently selected from phenyl or naphthyl; each R¹ is independently hydrogen, an alkyl group, an aryl group, or both R¹ together with the carbon atoms to which R¹ are attached form a cycloalkylene group, an arylene group, two or more cycloalkylene groups connected by a single bond or an alkyl group, two or more arylene groups connected by a single bond or an alkyl group, or a cycloalkylene and an arylene group connected by a single bond or an alkyl group; each R² is independently hydrogen or an alkyl group; each R³ is independently an alkyl group, an alkoxy group, an alkaryl group, an alkylsilyl ether group, or a halide; each R⁴ is independently an alkyl group, an alkoxy group, an alkaryl group, an alkylsilyl ether group, or a halide; R⁹ is selected from hydrogen, an optionally substituted alkyl group, an optionally substituted aryl group, hydroxyl or an amino group; each n is selected from 0 to 4; m is selected from 0 to 4; each R⁶ is independently selected from hydrogen, an optionally substituted alkyl group, an optionally substituted aryl group, or an optionally substituted heteroaromatic group; and each R⁷ is independently selected from the group consisting of hydrogen, an alkyl group, an ester-containing alkyl group or a carboxylic acid containing alkyl group.

The compound of Formula (III) is an isomer of farnesene. The compound of Formula (III) is B-farnesene, which may be referred to as 7,11-dimethyl-3-methylene-1,6,10-dodecatriene. The compound of Formula (III) may be (6E)-β-farnesene, which may be referred to as 7,11-dimethyl-3-methylene-1,6E,10-dodecatriene. The compound of Formula (III) may be the compound with CAS number [18794-84-8] or the compound with CAS number [77129-48-7]. In some examples, the stereochemistry of the double bond (i.e., the double bond between C6 and C7 of the compound of Formula (III)) remains unchanged during the method for making a compound of Formula (I), a compound of Formula (II) or a mixture thereof.

The compound of formula (III) may be replaced by a mixture comprising optionally a further isomer of farnesene, e.g., a-farnesene (3,7,11-trimethyldodeca-1,3,6,10-tetraene), which includes (E,E)-α-farnesene (CAS 502-61-4) and (Z,Z)-α-farnesene (CAS 18794-84-8).

The method for making a compound of Formula (I), a compound of Formula (II) or a mixture thereof may be a method for making a mixture of a compound of Formula (I) and a compound of Formula (II). In some examples, the mixture of a compound of Formula (I) and a compound of Formula (II) may be about a 99:1 to about 1:99 mixture, for example, about a 90:1 to about a 1:90 mixture, about a 80:20 to 20:80 mixture, about a 60:40 to about a 40:60 mixture, for example, about a 55:45 to about a 45:50 mixture or about a 50:50 mixture. In some examples, the mixture may be about a 1:1 mixture of compounds of Formula (I) and Formula (II).

The method for making a compound of Formula (I), a compound of Formula (II) or a mixture thereof may comprise contacting a compound of Formula (III) with a catalyst comprising a metal complexed with a salen ligand or a porphyrin ligand, wherein the salen ligand has Formula (IV), and wherein the porphyrin ligand has Formula (XI). The metal may be a transition metal, for example, manganese, chromium, iron, nickel, cobalt, titanium, vanadium, ruthenium or osmium. The metal may be manganese.

The catalyst comprising a metal complexed with a salen ligand or a porphyrin ligand may comprise or consist of manganese complexed with a salen ligand or a porphyrin ligand. The catalyst comprising a metal complexed with a salen ligand or a porphyrin ligand may comprise or consist of manganese (III) complexed with a salen ligand or a porphyrin ligand.

The salen ligand is a ligand of Formula (IV), wherein each ring A is independently selected from phenyl or naphthyl; each R¹ is independently hydrogen, an alkyl group, or an aryl group, or both R¹ together with the carbon atoms to which R¹ are attached form a cycloalkylene group, an arylene group, two or more cycloalkylene groups connected by a single bond or an alkyl group, two or more arylene groups connected by a single bond or an alkyl group, or a cycloalkylene and an arylene group connected by a single bond or an alkyl group; each R² is independently hydrogen or an alkyl group; each R³ is independently an alkyl group, an alkoxy group, an alkaryl group, an alkylsilyl ether group, or a halide; each R⁴ is independently an alkyl group, an alkoxy group, an alkaryl group, an alkylsilyl ether group, or a halide; R⁹ is selected from hydrogen, an optionally substituted alkyl group, an optionally substituted aryl group, hydroxyl or an amino group; each n is selected from 0 to 4, for example, 1 to 4; m is selected from 0 to 4.

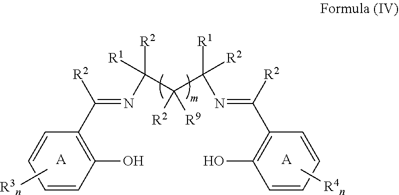

In some examples, each ring A is independently selected from phenyl or naphthyl. In some examples, each ring A is a phenyl group.

In some examples, each R¹ is independently hydrogen, a C1 to C10 alkyl group, or a C5 to C10 aryl group or both R¹ together with the carbon atoms to which R¹ are attached form a C5 to C10 cycloalkylene group, a C5 to C10 arylene group, two or more C5 to C10 cycloalkylene groups connected by a single bond or a C1 to C5 alkyl group (for example, a methyl group), two or more C5 to C10 arylene groups connected by a single bond or a C1 to C5 alkyl group (for example, a methyl group) or a C5 to C10 cycloalkylene group and a C5 to C10 arylene group connected by a single bond or a C1 to C5 alkyl group (for example, a methyl group). In some examples, each R¹ is the same or different or both R¹ together with the carbon atoms to which R¹ are attached form a cycloalkylene group. In some examples, each R¹ is the same. In some examples, both R¹ together with the carbon atoms to which R¹ are attached form a cycloalkylene group, wherein m is optionally 0.

In some examples, each R¹ is independently hydrogen, a C1 to C10 alkyl group, or a C5 to C10 aryl group or both R¹ together with the carbon atoms to which R¹ are attached form a C5 to C10 cycloalkylene group. In some examples, both R¹ together with the carbon atoms to which R¹ are attached form a C5 to C10 cycloalkylene group and wherein m is 0. In some examples, each R¹ is independently a C1 to C6 alkyl group, or a C5 or C6 aryl group or both R¹ together with the carbon atoms to which R¹ are attached are a C5 to C7 cycloalkylene group and m is optionally 0. In some examples, both R¹ together with the carbon atoms to which R¹ are attached form a cyclohexylene group and m is 0. In some examples, each R² is independently hydrogen or a C1 to C10 alkyl group. In some examples, each R² is the same or different. In some examples, each R² is the same. In some examples, each R² is hydrogen.

In some examples, each R³ is independently a C1 to C20 alkyl group, a C1 to C20 alkoxy group, a C1 to C20 alkaryl group, a C1 to C10 alkyl silyl ether group or a halide. In some examples, each R³ is independently methyl, ethyl, propyl, butyl (e.g., tert-butyl), pentyl, hexyl, methoxy, ethoxy, propoxy, butyoxy (e.g., tert-butyoxy), pentoxy, hexoxy, (butylphenyl)propyl (e.g., (tert-butylphenyl)propyl), trimethylsilyl ether, triethylsilyl ether, tripropylsilyl ether (e.g., tri(isopropyl)silyl ether), tributylsilyl ether, tripentylsilyl ether, trihexylsilyl ether, iodide, bromide, chloride, or fluoride.

In some examples, each R⁴ is independently a C1 to C20 alkyl group, a C1 to C20 alkoxy group, a C1 to C20 alkaryl group, a C1 to C10 alkyl silyl ether group or a halide. In some examples, each R⁴ is independently methyl, ethyl, propyl, butyl (e.g., tert-butyl), pentyl, hexyl, methoxy, ethoxy, propoxy, butyoxy (e.g., tert-butyoxy), pentoxy, hexoxy, (butylphenyl)propyl (e.g., (tert-butylphenyl)propyl), trimethylsilyl ether, triethylsilyl ether, tripropylsilyl ether (e.g., tri(isopropyl)silyl ether), tributylsilyl ether, tripentylsilyl ether, trihexylsilyl ether, iodide, bromide, chloride, or fluoride.

In some examples, each n is selected from 1 to 4. In some examples, n is 0. In some examples, each n is independently, 1, 2, 3 or 4. In some examples, each n is the same or different. In some examples, each n is the same. In some examples, each n is 2.

In some examples, each R³, is the same or different. In some examples, each R³, is the same. In some examples, each R⁴, is the same or different. In some examples, each R⁴, is the same. In some examples R³ is the same as R⁴.

In some examples, R⁹ is selected from hydrogen, an optionally substituted C1 to C10 alkyl group, an optionally substituted C6 to C14 aryl group, hydroxyl or an amino group.

In some examples, m is selected from 0 to 1. In some examples, m is 0.

In some examples, the salen ligand has Formula (IV). In some examples, the salen ligand has Formula (IV) and m is 0. In some examples, the salen ligand has Formula (IV), each ring A is phenyl and m is 0. In some examples, the salen ligand has Formula (IV-1) wherein each R¹ is as defined herein, each R² is as defined herein; each R³ is as defined herein; each R⁴ is as defined herein; and n is as defined herein.

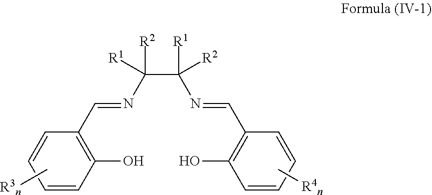

In some examples, the salen ligand has Formula (IV-2) wherein each R¹ is as defined herein, each R² is as defined herein; each R³ is as defined herein; each R⁴ is as defined herein; and n is as defined herein.

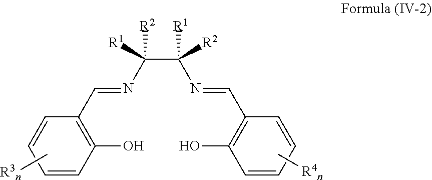

In some examples, the salen ligand has Formula (IV-i), for example, Formula (IV-i-1), wherein each R⁴ is independently selected from a group as defined herein and each R⁴ is independently selected from a group as defined herein. In some examples, the salen ligand has Formula (IV-i), for example, Formula (IV-i-1), wherein each R³ is the same or different and each R⁴ is the same or different. In some examples, the salen ligand has Formula (IV-i), for example, Formula (IV-i-1), wherein each R³ is the same and/or each R⁴ is the same.

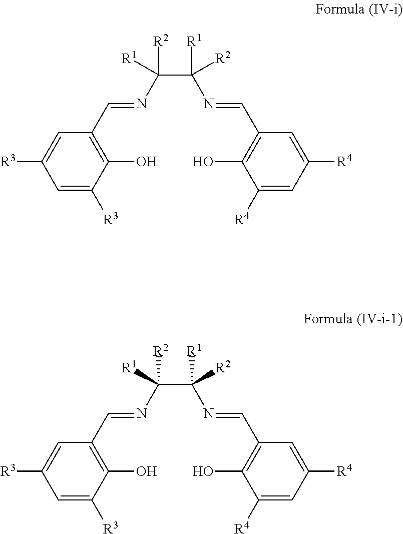

In some examples, the salen ligand has Formula (IV-ii) or Formula (IV-iii), wherein R³ and R⁴ are as defined herein.

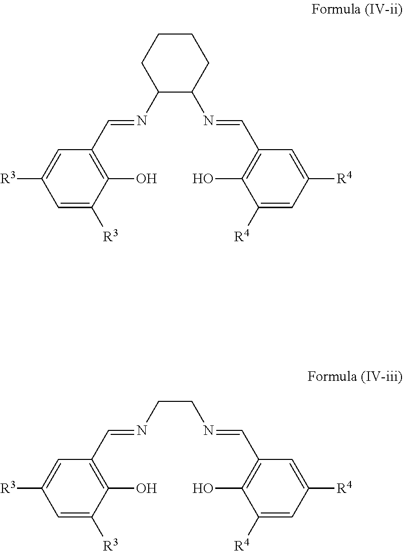

In some examples, the salen ligand has Formula (IV-iv) or Formula (IV-v).

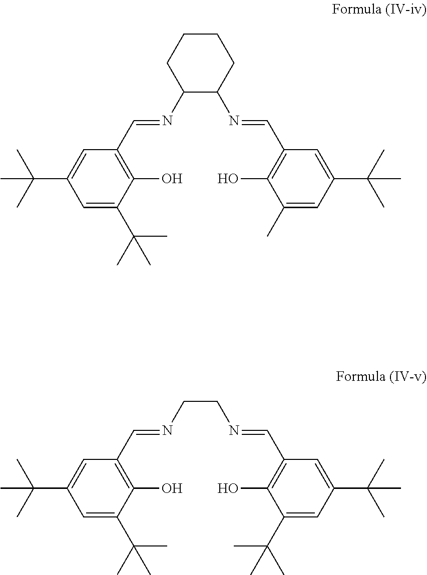

In some examples, the catalyst comprising a metal complexed with a salen ligand is Jacobsen's catalyst, wherein Jacobsen's catalyst has Formula (IVa) or Formula (IVb) or is a mixture of Formula (IVa) and Formula (IVb), which may be an optically active mixture or a racemic mixture.

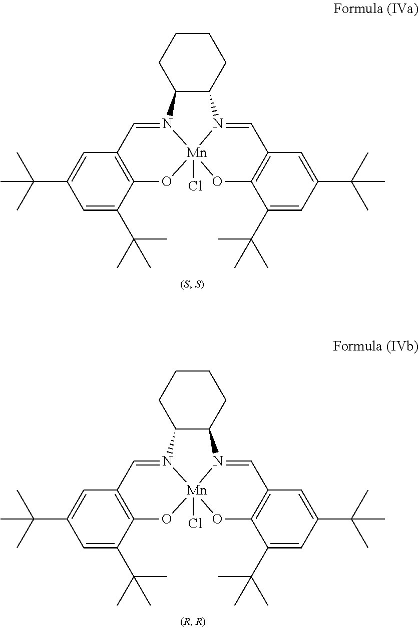

In some examples, the catalyst comprising a metal complexed with a salen ligand is a compound of Formula (IVc) (which includes an optically active mixture or a cis/trans mixture), or a compound of Formula (IVd).

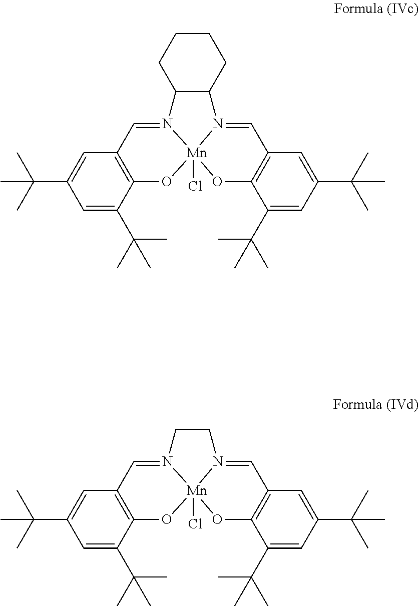

In some examples, the porphyrin ligand is a ligand of Formula (XI), wherein each R⁶ is independently selected from hydrogen, an optionally substituted alkyl group, an optionally substituted aryl group, or an optionally substituted heteroaromatic group; and each R⁷ is independently selected from hydrogen, an alkyl group, an ester-containing alkyl group or a carboxylic acid containing alkyl group.

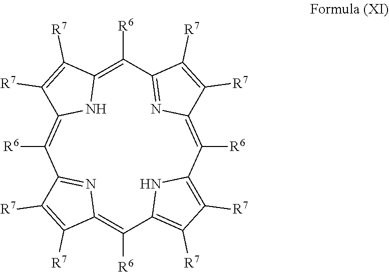

In some examples, the porphyrin ligand is a ligand of Formula (XI), wherein each R⁶ is independently selected from hydrogen, an optionally substituted alkyl group, an optionally substituted aryl group, or an optionally substituted heteroaromatic group; and each R⁷ is independently selected from hydrogen, an alkyl group, an ester-containing alkyl group or a carboxylic acid containing alkyl group; wherein an optionally substituted alkyl group is an alkyl group wherein one or more hydrogen atoms is replaced by an alkyl or aryl group, amino group, a hydroxy group an alkoxy group a carboxylic acid or a salt thereof, a carboxylic acic ester, halides such as chlorides, fluorides, a sulfonic acid or a salt thereof, a nitro group; wherein an optionally substituted aryl group is an aryl group wherein one or more hydrogen atoms is replaced by an alkyl or aryl group, amino group, a hydroxy group an alkoxy group a carboxylic acid or a salt thereof, a carboxylic acic ester, halides such as chlorides, fluorides, a sulfonic acid or a sald thereof, a nitro group; wherein an optionally substituted heteroaromatic group is a heteroaromatic group wherein one or more hydrogen atoms is replaced by an alkyl or aryl group. All substituents can form further ring systems.

In some examples, the porphyrin ligand is a ligand of Formula (XI), each R⁶ is independently selected from hydrogen, an alkyl group, an aryl group, or a heteroaromatic group; and each R⁷ is independently selected from hydrogen, an alkyl group, an ester-containing alkyl group or a carboxylic acid containing alkyl group. the porphyrin ligand is a ligand of Formula (XI), each R⁶ is independently selected from hydrogen, a C1 to C10 alkyl group, a C6 to C14 aryl group, or a C3 to C10 heteroaromatic group; and each R⁷ is independently selected from hydrogen, an C1 to C10 alkyl group, an ester-containing C2 to C10 alkyl group or a carboxylic acid containing C1 to C10 alkyl group.

In some examples, each R⁶ is the same or different and each R⁷ is the same or different. In some examples, each R⁶ is the same and each R⁷ is the same or different. In some examples, each R⁶ is the same or different and each R⁷ is the same. In some examples, each R⁶ is the same and each R⁷ is the same.

In some examples, each R⁶ is hydrogen or an aryl group, for example, a C6 to C10 aryl group. In some examples, each R⁷ is hydrogen. In some examples, each R⁶ is hydrogen or an aryl group, for example, a C6 to C10 aryl group and each R⁷ is hydrogen. In some examples, each R⁶ is an aryl group, for example, a C6 to C10 aryl group and each R⁷ is hydrogen. In some examples, each R⁶ is phenyl and each R⁷ is hydrogen.

In some examples, the porphyrin ligand is a ligand of Formula (XI-1), wherein each R⁶ is as defined herein.

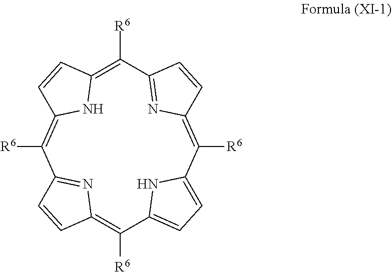

In some examples, the porphyrin ligand is a ligand of Formula (XI-1), wherein each R⁶ is independently selected from hydrogen, an optionally substituted alkyl group, an optionally substituted aryl group, or an optionally substituted heteroaromatic group. In some examples, the porphyrin ligand is a ligand of Formula (XI-1), wherein each R⁶ is independently a C6 to C10 aryl group.

In some examples, the porphyrin ligand is a ligand of formula (XI-2). In some examples, the catalyst comprising a metal complexed with a porphyrin ligand is a catalyst of Formula (XIa).

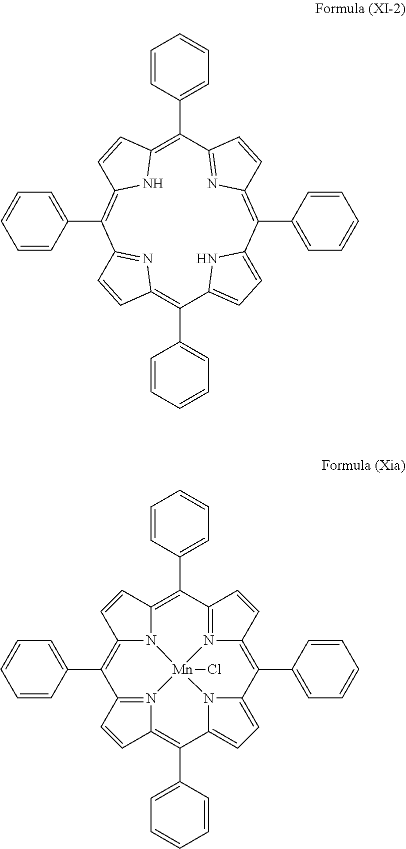

In some examples, contacting the compound of Formula (III) with a catalyst comprising a metal complexed with a salen ligand or a porphyrin ligand may comprise contacting the compound of Formula (III) with a catalyst comprising a metal complexed with a salen ligand or a porphyrin ligand and an oxidant. In some examples, contacting the compound of Formula (III) with a catalyst comprising a metal complexed with a salen ligand or a porphyrin ligand may comprise contacting the compound of Formula (III) with a catalyst comprising a metal complexed with a salen ligand or a porphyrin ligand and a co-catalyst or additive. In some examples, contacting the compound of Formula (III) with a catalyst comprising a metal complexed with a salen ligand or a porphyrin ligand may comprise contacting the compound of Formula (III) with a catalyst comprising a metal complexed with a salen ligand or a porphyrin ligand, an oxidant and a co-catalyst or additive.

In some examples, contacting the compound of Formula (III) with a catalyst comprising a metal complexed with a salen ligand may comprise contacting the compound of Formula (III) with a catalyst comprising a metal complexed with a salen ligand and an oxidant. In some examples, contacting the compound of Formula (III) with a catalyst comprising a metal complexed with a salen ligand may comprise contacting the compound of Formula (III) with a catalyst comprising a metal complexed with a salen ligand and a co-catalyst or additive. In some examples, contacting the compound of Formula (III) with a catalyst comprising a metal complexed with a salen ligand may comprise contacting the compound of Formula (III) with a catalyst comprising a metal complexed with a salen ligand, an oxidant and a co-catalyst or additive.

In some examples, the oxidant may be selected from a hypochlorite, a peroxide, a periodate and molecular oxygen. In some examples, the hypochlorite may be sodium hypochlorite (NaOCl). In some examples, the peroxide may be hydrogen peroxide, anhydrous urea-hydrogen peroxide, dimethyldioxirane, or tert-butylhydroperoxide (tBuOOH). In some examples, the periodate may be IO₄⁻ or IO₆⁵⁻. In some examples, the oxidant may be sodium hypochlorite or hydrogen peroxide.

In some examples, the co-catalyst or additive may be any compound capable of axial coordination with the metal of the catalyst. In some examples, the co-catalyst or additive may be an organonitrile, an N-oxide ligand, an alkylsulfonate, a nitrogen-containing heterocycle, a salt (e.g., an acetate, a carboxylate, a carbonate, or a quaternary amine salt), a phosphate, a phosphine oxide, a phosphoramide, an aldehyde (e.g, pivaldehyde) or a mixture thereof. An aldehyde, such as pivaldehyde, may be used in combination with molecular oxygen as the oxidant and act as, for example, a reductant for molecular oxygen.

The organonitrile may be acetonitrile. The N-oxide ligand may be a tertiary amine N-oxide (such as trimethylamine N-oxide, p-CN-N,N-dimethylaniline N-oxide, N-methyl morpholine N-oxide (NMO),), or a pyridine N-oxide (e.g., pyridine N-oxide, 4-tert-butylpyridne N-oxide, 4-methylpyridine N-oxide, or 4-phenylpyridine N-oxide. The alkylsulfonate may be trifluoromethane sulfonate. The nitrogen-containing heterocycle may be pyridine, an alkylpyridine (such as 4-tert-butylpyridine or dimethylaminopyridine) or an imidazole. In some examples, the acetate may be sodium acetate (NaOAc). In some examples, the carboxylate may be sodium benzoate (NaOCOPh). In some examples, the carbonate may be sodium bicarbonate (NaHCO₃). In some examples, the quaternary amine salt may be NH₄BF₄, NH₄F, NH₄ClO₄, NH₄PF₆, NH₄Oac, NH₄OCHO a salt of a quinine or a derivative thereof, a salt of cinchonidine or a derivative thereof, a salt of a cinchona alkaloide or a derivative thereof, a salt of quinidine or a derivative thereof, or a salt of ephedrine or a derivative thereof. In some examples, the phosphate may be Na₃PO₄. The phosphine oxide may be Ph₃PO. The phorphoramide may be hexamethylphosphoramide.

In some examples, the co-catalyst or additive may be trimethylamine N-oxide or a mixture of NH₄BF₄ and Na₃PO₄.

In some examples, the method comprises contacting the compound of Formula (III) with a catalyst comprising a metal complexed with a salen ligand in a solvent. In some examples, the solvent may be a solvent capable of axial coordination with the metal of the catalyst. In some examples, the solvent is capable of axial coordination with the metal of the catalyst and no co-catalyst or additive is included. In some examples, the solvent is selected from the group consisting of phenyl chloride, toluene, methyl tert-butyl ether, xylene, dichloroethane, dichloromethane, cyclohexane, cyclohexane, dimethyl isosorbide, dimethyl carbonate, dimethoxyethane, substituted N-methyl pyrrolidones (e.g. N-methyl-pyrrolidone, N-butyl-pyrrolidone, N-octyl-pyrrolidone), (trifluoromethyl)benzene, benzene, substituted esters (e.g. ethyl acetate, propyl acetate, butyl acetate, methyl pivalate, ethyl pivalate), substituted dialkyl ethers (e.g. diethyl ether, dipropyl ether, dibutyl ether), methyl tetrahydrofuran, dioxane, dimethoxy ethane, dimethyl diglycol ether, dimethyl tetraglycol ether, substituted alkanes (e,g, pentane, hexane, heptane, octane, decane, dodecane), and cyclohexane. In some examples, the solvent is methanol, toluene, benzene, xylene, chlorobenzene, ethyl acetate, dichloromethane or (trifluoromethyl)benzene. In some examples, no organic solvent is used.

In some examples, contacting the compound of Formula (III) with a catalyst comprising a metal complexed with a salen ligand or porphyrin ligand comprises contacting the compound of Formula (III) with a catalyst comprising a metal complexed with a salen ligand and aqueous NaOCl and a solvent.

In some examples, contacting the compound of Formula (III) with a catalyst comprising a metal complexed with a salen ligand or porphyrin ligand comprises contacting the compound of Formula (III) with a catalyst comprising a metal complexed with a salen ligand and either a) aqueous NaOCl, MesNO and PhMe; or b) aqueous H₂O₂, NH₄BF₄, Na₃PO₄ and MeOH or c) aqueous NaOCl and toluene, benzene, chlorobenzene, ethyl acetate, or (trifluoromethyl)benzene.

In some examples, the method for making a compound of Formula (I), a compound of Formula (II) or a mixture thereof comprises contacting a compound of Formula (III) with a catalyst comprising a metal complexed with a salen ligand or a porphyrin ligand at a temperature of room temperature or below, for example, from about 0° C. to room temperature, or from about 5° C. to room temperature. Room temperature may be a temperature of from about 20° C. to about 25° C.

In some examples, the method for making a compound of Formula (I), a compound of Formula (II) or a mixture thereof comprises contacting a compound of Formula (III) with a catalyst comprising a metal complexed with a salen ligand or a porphyrin ligand at a temperature of room temperature (about 20° C. to about 25° C.) or above, for example, from about room temperature up to 150° C. (e.g. up to 120° C. or up to 60° C.), which includes a temperature of about 25° C. to about 60° C.

In some examples, the method for making a compound of Formula (I), a compound of Formula (II) or a mixture thereof comprises contacting the compound of Formula (III) with a catalyst at a temperature which is the reflux temperature of the respective solvent used.

In some examples, the method for making a compound of Formula (I), a compound of Formula (II) or a mixture thereof comprises contacting a compound of Formula (III) with a catalyst comprising a metal complexed with a salen ligand or a porphyrin ligand for 1 hour or more, for example, 5 hours-120 hours (including 10, 12, 18, 24, 30, 36,42, 48, 54, 60, 66, 72, 78, 84, 90, 96, 102, 108, 114 hours) or more.

In some examples, the method for making a compound of Formula (I), a compound of Formula (II) or a mixture thereof comprises contacting a compound of Formula (III) with a catalyst comprising a metal complexed with a salen ligand or a porphyrin ligand for 120 hours or less, for example, up to 114 hours (which includes 5, 10, 12, 18, 24, 30, 36,42, 48, 54, 60, 66, 72, 78, 84, 90, 96, 102, 108 hours)or less.

In some examples, the method produces a compound of Formula (I). In some examples, the method produces a compound of Formula (II). In some examples, the method produces a mixture of the compounds of Formula (I) and (II).

In some examples, the method has a conversion of less than 100%, that is, less than 100% of the compound of Formula (III) reacts. In some examples, at least some of the compound of Formula (III) remains after the method has been performed. In some examples, the method produces a mixture of a compound of Formula (I) and the unreacted compound of Formula (III). In some examples, the method produces a mixture of a compound of Formula (II) and the unreacted compound of Formula (III). In some examples, the method produces a mixture of a compound of Formula (I), a compound of Formula (II) and the unreacted compound of Formula (III). In some examples, unreacted compound of Formula (III) is separated from the product mixture. In some examples, unreacted compound of Formula (III) remains part of the product mixture.

In some examples, the method produces a mixture of a compound of Formula (I) and a compound of Formula (II) and the method further comprises separating the compounds of Formula (I) and (II). In some examples, the compounds of Formula (I) and (II) are separated by distillation. Compounds of Formula (I) and Formula (II) degrade on silica gel and have similar boiling points (Formula (I): 83° C./0.03 mbar; Formula (II): 78° C./0.03 mbar). However, it was surprisingly found that compounds of Formula (I) and (II) can be separated by distillation in spite of their very similar boiling points. In some examples, separation by distillation also removes remaining unreacted reagent from the product mixture.

In some examples, the compound of Formula (I) is E-Formula (I). In some examples, the compound of Formula (II) is E-Formula (II).

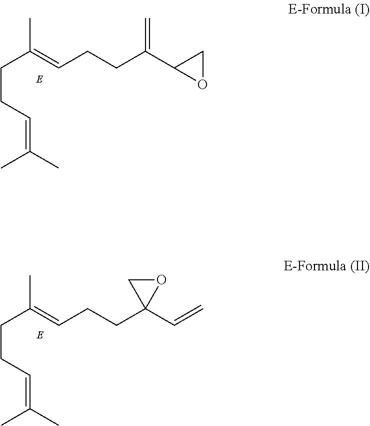 [SMILES: C=C(CC/C=C(\C)CCC=C(C)C)C1CO1 ; C=CC1(CC/C=C(\C)CCC=C(C)C)CO1]

In some examples, the compound of Formula (I) is farnesene 1,2-epoxide, which is the compound with CAS number [83637-40-5].

In some examples, the compound of Formula (II) is farnesene exo-epoxide, which is the compound with CAS number [1404220-65-0].

In some examples the method produces a mixture of a compound of Formula (I) and a compound of Formula (I) wherein the compound of Formula (II) is provided in excess, e.g. the compound of Formula (II) and compound of Formula (I) are produced in a ratio of 51:49 to 99:1 (which includes a range of 55:45 to 60:40).

### Method of Separation of a Mixture of Compounds of Formulae (I) and (II)

In an aspect, there is provided a method of separation of a mixture of a compound of Formula (I) and a compound of Formula (II), the method comprising the distillation of the mixture of a compound of Formula (I) and a compound of Formula (II).

In some examples, the distillation comprises separation under vacuum or wiped film distillation (which may be referred to as short path distillation). In some examples, the method of separation of a mixture of a compound of Formula (I) and a compound of Formula (II) comprises the vacuum distillation of the mixture of a compound of Formula (I) and a compound of Formula (II).

In some examples, vacuum distillation comprises distillation at a reduced pressure of up to about 1 mbar, for example, up to about 0.7 mbar, up to about 0.5 mbar, up to about 0.3 mbar, up to about 0.2 mbar, up to about 0.1 mbar, up to about 0.09 mbar, up to about 0.08 mbar, up to about 0.07 mbar, up to about 0.06 mbar, up to about 0.05 mbar, up to about 0.04 mbar, up to about 0.03 mbar, up to about 0.02 mbar, up to about 0.01 mbar. In some examples, vacuum distillation comprises distillation at a reduced pressure of from about 0.01 mbar to 0.5 mbar, for example, from about 0.02 mbar to about 0.4 mbar, about 0.03 mbar to about 0.4 mbar, about 0.04 mbar to about 0.2 mbar, about 0.05 mbar to about 0.1 mbar, about 0.03 mbar to about 0.02 mbar. In some examples, the vacuum distillation may be performed at a pressure of about 0.03 mbar (3 Pa). At 0.03 mbar, the boiling point of a E-Formula (I) is 83° C. and the boiling point to of E-Formula (II) is 78° C.

### Method for Making Compounds of Formulae (V) and (VI)

In another aspect, there is provided a method for making a compound of Formula (V), a compound of Formula (VI) or a mixture thereof,

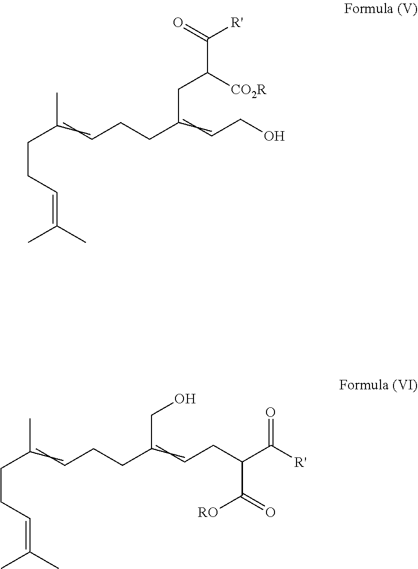

wherein the method comprises contacting a compound of Formula (I), a compound of Formula (II) or a mixture thereof, respectively, with a compound of Formula (VII) and a palladium catalyst, wherein R is selected from the group consisting of hydrogen and a C1 to C6 alkyl group; and R′ is selected from the group consisting of a C1 to C6 alkyl group and an OR group.

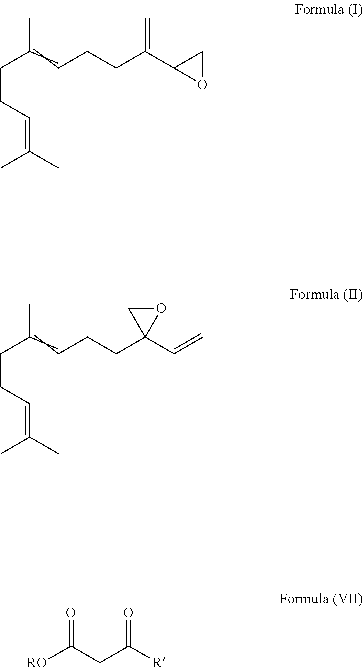

In some examples, the method for making a compound of Formula (V), a compound of Formula (VI) or a mixture thereof, is a method for making a compound of Formula (V), wherein the method comprises contacting a compound of Formula (I) with a compound of Formula (VII) and a palladium catalyst, wherein R is selected from the group consisting of hydrogen and C1 to C6 alkyl groups; and R′ is selected from the group consisting of a C1 to C6 alkyl group and an OR group (for example, as summarised in the schematic below).

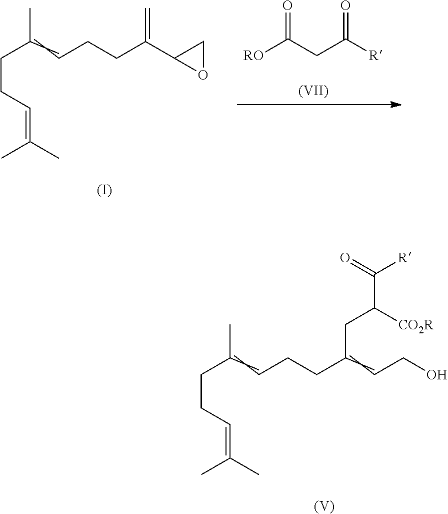

In some examples, the method for making a compound of Formula (V), a compound of Formula (VI) or a mixture thereof is a method for making a compound of Formula (VI), wherein the method comprises contacting a compound of Formula (II) with a compound of Formula (VII) and a palladium catalyst, wherein R is selected from the group consisting of hydrogen and C1 to C6 alkyl groups; and R′ is selected from the group consisting of a C1 to C6 alkyl group and an OR group (for example, as summarised in the schematic below).

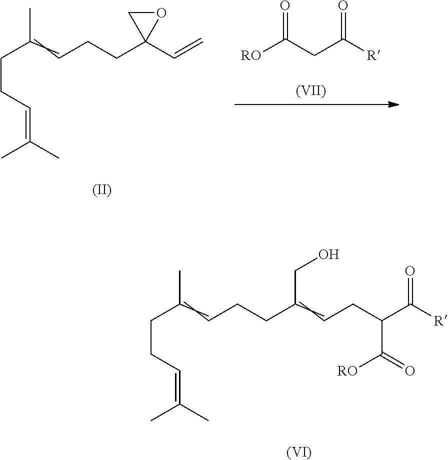

In some examples, the method for making a compound of Formula (V), a compound of Formula (VI) or a mixture thereof is a method for making a mixture of a compound of Formula (V) and a compound of Formula (VI), wherein the method comprises contacting a mixture of a compound of Formula (I) and a compound of Formula (II) with a compound of Formula (VII) and a palladium catalyst, wherein R is selected from the group consisting of hydrogen and C1 to C6 alkyl group; and R′ is selected from the group consisting of a C1 to C6 alkyl group and an OR group.

In some examples, the stereochemistry of the double bond in the reagent (i.e., the double bond between C6 and C7 of the compound of Formula (III)) remains unchanged during the method for making a compound of Formula (V), a compound of Formula (VI) or a mixture thereof.

In some examples, the method produces a mixture of E,E- and E,Z-Formula (V) and/or a mixture of E,E- and E,Z-Formula (VI).

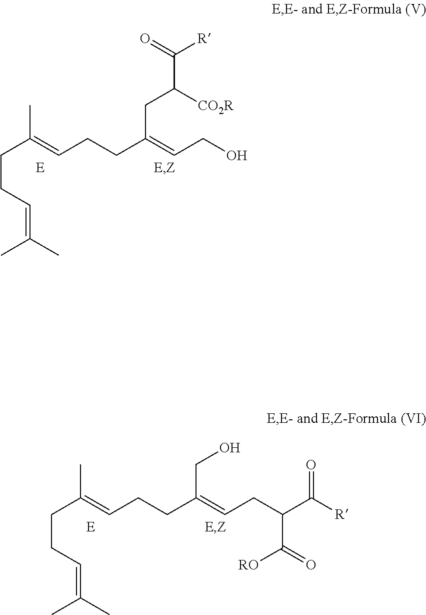

In some examples, the palladium catalyst may be a Pd(0) catalyst, a Pd(II) catalyst or a supported heterogeneous Pd catalyst. In some examples, the palladium catalyst may be selected from Pd(PPh₃)₄, Pd₂(dba)₃ (wherein dba is dibenzylideneacetone), solvated Pd₂(dba)₃ (such as Pd₂(dba)₃·dba and Pd₂(dba)₃·CHCl₃), Pd(OAc)₂ (wherein Ac is acetyl), Pd(acac)₂ (wherein acac is acetylacetonate), Pd(MeCN)₂Cl₂, Pd(COD)Cl₂ (wherein COD is 1,5-cyclooctadiene), [(C₃H₅)PdCl]₂, PdCl₂ or Pd/C. In some examples, the palladium catalyst is palladium (II) bis(acetylacetonate).

In some examples, the palladium catalyst may be a catalyst comprising palladium and a ligand or cation selected from the group consisting of PPh₃, dba, OAc, acac, MeCN, Cl, COD, C₃H₅, DPPF (wherein DPPF is 1,1′-ferrocenediyibis(diphenylphosphine) [CAS 12160-46-8]), DPPB (wherein DPPB is 1,4-bis(diphenylphosphino)butane [CAS 7688-25-7]), BINAP (wherein BINAP is rac-BINAP wherein BINAP is 2,2′.

bis(diphenylphosphino)-1,1′-binaphthalene [CAS 98327-87-8]), DPEphos (wherein DPEphos is bis[(2-diphenyiphosphino)phenyl] ether [CAS 166330-10-5]), tris(4-methoxyphenyl)phosphine [CAS 855-38-9], tris(2-furyl)phosphine [CAS 5518-52-5], Triethyl phosphite P(OEt)₃ [CAS 122-52-1], and triisopropyl phosphite P(iPrO): [CAS 116-17-6].

The compound of Formula (VII) is an oxoalkanoic acid, an oxoalkanoic ester, malonic acid or a malonic ester. The compound of Formula (VII) may be an oxoalkanoic ester or a malonic ester.

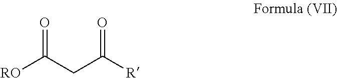

In some examples, R is selected from the group consisting of hydrogen and C1 to C6 alkyl groups. In some examples, R is selected from the group consisting of hydrogen, methyl, ethyl, propyl and isopropyl. In some examples, R is selected from the group consisting of hydrogen, methyl and ethyl. In some examples, R is methyl.

In some examples, R′ is selected from the group consisting of a C1 to C6 alkyl group and an OR group. In some examples, R′ is selected from the group consisting of a C1 to C6 alkyl group, hydroxyl and a C1 to C6 alkoxy group. In some examples, R′ is selected from the group consisting of hydrogen, methyl, ethyl, propyl, isopropyl, hydroxyl, methoxy, ethoxy, propoxy and isopropoxy. In some examples, R′ is selected from the group consisting of hydrogen, methyl, ethyl, propyl, isopropyl. In some examples, R′ is hydroxyl or a methyl group. In some examples, R′is a methyl group.

In some examples, the compound of Formula (VII) is a compound of Formula (VIIa). In some examples, the method comprises contacting a compound of Formula (I), a compound of Formula (II) or a mixture thereof with a compound of Formula (VIIa) and a palladium catalyst. In some examples, R may be as defined above, for example, R may be selected from the group consisting of hydrogen and a C1 to C6 alkyl group (for example, a methyl group).

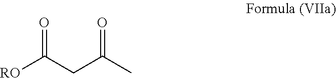

In some examples, the method for making a compound of Formula (V), a compound of Formula (VI) or a mixture thereof comprises a method for making a compound of Formula (V). In some examples, the method for making a compound of Formula (V), a compound of Formula (VI) or a mixture thereof comprises a method for making a compound of Formula (VI). In some examples, the method for making a compound of Formula (V), a compound of Formula (VI) or a mixture thereof comprises a method for making a mixture of a compound of Formula (V) and a compound of Formula (VI).

In some examples, the method for making a compound of Formula (V), a compound of Formula (VI) or a mixture thereof comprises contacting the product of a method for making a compound of Formula (I), a compound of Formula (II) or a mixture thereof with a compound of Formula (VII) and a palladium catalyst. In some examples, the method for making a compound of Formula (V), a compound of Formula (VI) or a mixture thereof comprises making a compound of Formula (I), a compound of Formula (II) or a mixture thereof by a method described herein; and contacting the compound of Formula (I), the compound of Formula (II) or the mixture thereof with a compound of Formula (VII) and a palladium catalyst (for example, as summarised in the schematic below). In some examples, unreacted compound of Formula (III) remains in the reaction mixture during the method for making a compound of Formula (V), a compound of Formula (VI) or a mixture thereof.

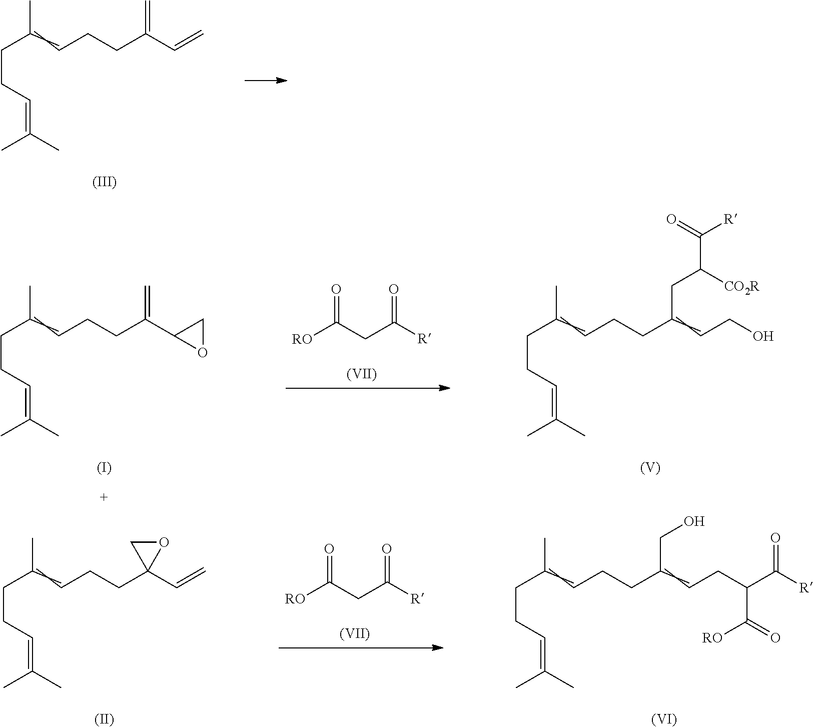

In some examples, the method for making a mixture of a compound of Formula (V) and a compound of Formula (VI) comprises contacting the product of a method for making a mixture of a compound of Formula (I) and a compound of Formula (II) with a compound of Formula (VII) and a palladium catalyst. In some examples, the method for making a mixture of a compound of Formula (V) and a compound of Formula (VI) comprises making a mixture of a compound of Formula (I) and a compound of Formula (II) by a method described herein; and contacting the mixture of a compound of Formula (I) and a compound of Formula (II) with a compound of Formula (VII) and a palladium catalyst. In some examples, unreacted compound of Formula (III) remains in the reaction mixture during the method for making a mixture of a compound of Formula (V) and a compound of Formula (VI).

In some examples, the method for making a compound of Formula (V) comprises contacting the product of a method for making a compound of Formula (I) with a compound of Formula (VII) and a palladium catalyst. In some examples, the method for making a compound of Formula (V) comprises making a compound of Formula (I) by a method described herein and contacting the compound of Formula (I) with a compound of Formula (VII) and a palladium catalyst. In some examples, unreacted compound of Formula (III) remains in the reaction mixture during the method for making a compound of Formula (V).

In some examples, the method for making a compound of Formula (V) comprises making a mixture of a compound of Formula (I) and a compound of Formula (II) by a method described herein; separating the compound of Formula (I) from the mixture of a compound of Formula (I) and a compound of Formula (II); and contacting the compound of Formula (I) with a compound of Formula (VII) and a palladium catalyst. In some examples, the method for making a compound of Formula (V) comprises making a mixture of a compound of Formula (I) and a compound of Formula (II) by a method described herein; separating the compound of Formula (I) from the mixture by distillation to produce the compound of Formula (I); and contacting the compound of Formula (I) with a compound of Formula (VII) and a palladium catalyst. In some examples, unreacted compound of Formula (III) remains in the reaction mixture during the method for making a compound of Formula (V).

In some examples, the method for making a compound of Formula (VI) comprises contacting the product of a method for making a compound of Formula (II) with a compound of Formula (VII) and a palladium catalyst. In some examples, the method for making a compound of Formula (VI) comprises making a compound of Formula (II) by a method described herein and contacting the compound of Formula (II) with a compound of Formula (VII) and a palladium catalyst. In some examples, unreacted compound of Formula (III) remains in the reaction mixture during the method for making a compound of Formula (VI).

In some examples, the method for making a compound of Formula (VI) comprises making a mixture of a compound of Formula (I) and a compound of Formula (II) by a method described herein; separating the compound of Formula (II) from the mixture of a compound of Formula (I) and a compound of Formula (II); and contacting the compound of Formula (II) with a compound of Formula (VII) and a palladium catalyst. In some examples, the method for making a compound of Formula (VI) comprises making a mixture of a compound of Formula (I) and a compound of Formula (II) by a method described herein; separating the compound of Formula (II) from the mixture by distillation to produce the compound of Formula (II); and contacting the compound of Formula (II) with a compound of Formula (VII) and a palladium catalyst. In some examples, unreacted compound of Formula (III) remains in the reaction mixture during the method for making a compound of Formula (VI).

In some examples, the method for making a compound of Formula (V) or a compound of Formula (VI) may comprise making a mixture of a compound Formula (V) and a compound of Formula (VI) and separating the compound of Formula (V) from the compound of Formula (VI). In some examples, unreacted compound of Formula (III) remains in the reaction mixture during the method for making a compound of Formula (VI).

In some examples, the method for making a compound of Formula (V), a compound of Formula (VI) or a mixture thereof produces a mixture of E,E- and E,Z-isomers. In some examples, the method further comprises separating the E,E- and E,Z-isomers. In some examples, the E,E- and E,Z-isomers may be separated by chromatography, for example, flash chromatography.

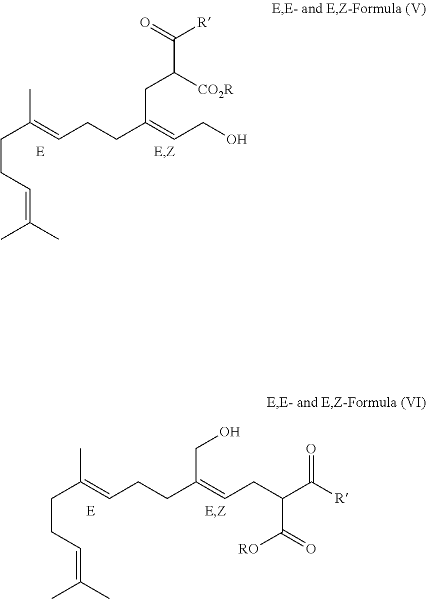

In some examples, the method for making a compound of Formula (V) produces a mixture of E,E- and E,Z-isomers. In some examples, the method further comprises separating the E,E- and E,Z-isomers. In some examples, the E,E- and E,Z-isomers may be separated by chromatography, for example, flash chromatography. In some examples, the E,Z isomer is the major isomer produced by the method. In some examples, the E,E isomer is the major isomer produced by the method. In some examples, the selection of reaction conditions, for example, ligand and or solvent used, affects which isomer is the major isomer produced by the method.

In some examples, the method for making a compound of Formula (VI) produces a mixture of E,E- and E,Z-isomers. In some examples, the method further comprises separating the E,E- and E,Z-isomers. In some examples, the E,E- and E,Z-isomers may be separated by distillation or chromatography, for example, flash chromatography. In some examples, the E,Z-isomer is the major isomer produced by the method.

In some examples, the method for making a mixture of a compound of Formula (V) and a compound of Formula (VI) produces a mixture of E,E-Formula (V), E,Z-Formula (V), E,E-Formula (VI) and E,Z-Formula (VI). In some examples, the method further comprises separating the E,E- and E,Z-isomers and/or separating the compound of Formula (V) and the compound of Formula (VI). In some examples, the E,E- and E,Z-isomers and/or the compound of Formula (V) and the compound of Formula (VI) may be separated by chromatography, for example, flash chromatography.

In some examples, the method for making a compound of Formula (V), a compound of Formula (VI) or a mixture thereof comprises contacting a compound of Formula (I), a compound of Formula (II) or a mixture thereof with a compound of Formula (VII) and a palladium catalyst at room temperature or above, for example, a temperature of up to 50° C., up to 40° C. In some examples, the method for making a compound of Formula (V), a compound of Formula (VI) or a mixture thereof comprises contacting a compound of Formula (I), a compound of Formula (II) or a mixture thereof with a compound of Formula (VII) and a palladium catalyst at a temperature of from 20° C. to 50° C., for example, 25° C. to 45° C., 25° C. to 40° C., or 30° C. to 35° C.

In some examples, the method for making a compound of Formula (V), a compound of Formula (VI) or a mixture thereof comprises contacting a compound of Formula (I), a compound of Formula (II) or a mixture thereof with a compound of Formula (VII) and a palladium catalyst for at least 0.5 hours, for example, up to 72 hours (which includes 1, 5, 10, 12, 24, 30, 36, 42, 48, 54, 60, 66 hours) or even longer.

In some examples, the method for making a compound of Formula (V), a compound of Formula (VI) or a mixture thereof comprises contacting a compound of Formula (I), a compound of Formula (II), or a mixture thereof with a compound of Formula (VII) and a palladium catalyst for from about 0.5 h to about 72 h (which includes the following ranges: 1 -66, 5-60, 10-54, 12-48, 18-42, 24-36, and 30-36 hours.

In some examples, the method for making a compound of Formula (V), a compound of Formula (VI) or a mixture thereof comprises contacting a compound of Formula (I), a compound of Formula (II), or a mixture thereof with a compound of Formula (VII) and a palladium catalyst in the presence of a solvent. In some examples the solvent is selected from the group consisting of tetrahydrofuran, diethyl ether, methyl t-butyl ether, 1,2-dimethoxyethane, dioxane, 2-methyltetrahydrofuran, cyclopentyl methyl ether, ethyl acetate, dimethylformamide, dimethyl sulfoxide, acetonitrile, acetone, and chlorobenzene, or mixtures thereof.

Method for making compounds of Formulae (VIII) and (IX) from compounds of Formulae (V) and (VI)

In another aspect, there is provided a method for making a compound of Formula (VIII), a compound of Formula (IX) or a mixture thereof,

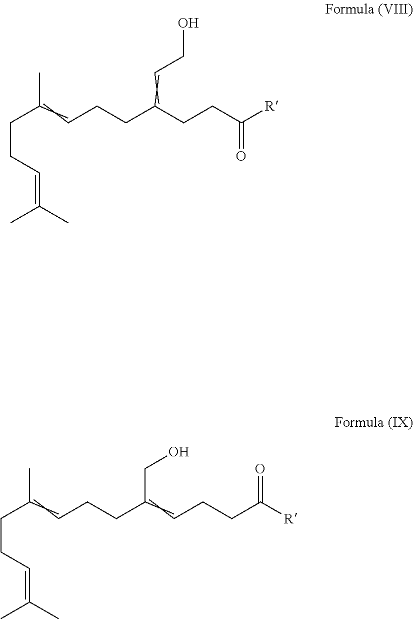

wherein the method comprises hydrolysis and decarboxylation of a compound of Formula (V), a compound of Formula (VI), or a mixture thereof,

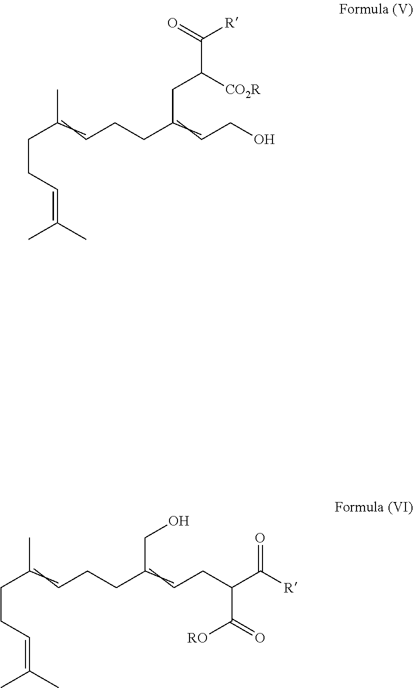

respectively, wherein R′ is selected from the group consisting of a C1 to C6 alkyl group and an OR group, and wherein R is selected from the group consisting of hydrogen or a C1 to C6 alkyl group.

In some examples, the E/Z ratio [of the double bonds] remains unchanged during the method of making a compound of Formula (VIII), a compound of Formula (IX), or a mixture thereof. In some examples, the reagent comprises a mixture of stereoisomers and the proportions of each stereoisomer remain unchanged during the method. In some examples, the reagent comprises a single isomer of the compound of Formula (V) and the E/Z ratio of the double bonds remains unchanged in the product. In some examples, the reagent comprises a single isomer of the compound of Formula (VI) and the E/Z ratio of the double bonds remains unchanged in the product. In some examples, the reagent comprises a single isomer of the compound of Formula (V) and a single isomer of the compound of Formula (VI), and the E/Z ratio of the double bonds remains unchanged in the product.

In some examples, the hydrolysis and decarboxylation may comprise contacting a compound of Formula (V), a compound of Formula (VI), or a mixture thereof with an acid or a base, wherein R′ is selected from the group consisting of a C1 to C6 alkyl group and an OR group, and wherein R is selected from the group consisting of hydrogen or a C1 to C6 alkyl group. In some examples, R′ and R are as defined for the compound of Formula (VII). In some examples, R′ is selected from the group consisting of hydrogen, methyl, ethyl, propyl and isopropyl and R is selected from the group consisting of hydrogen, methyl, ethyl, propyl and isopropyl.

In some examples, the compound of Formula (V) may be any compound of Formula (V) described herein. In some examples, the compound of Formula (VI) may be any compound of Formula (VI) described herein. In some examples, R′is methyl.

In some examples, the hydrolysis and decarboxylation may comprise heating the compound of Formula (V), the compound of Formula (VI) or the mixture thereof. In some examples, the hydrolysis and decarboxylation may comprise heating the compound of Formula (V), the compound of Formula (VI) or the mixture thereof in the presence of water, and/or an acid, a base, a polar solvent (such as DMSO), a salt, boric anhydride (B₂O₃). In some examples, the acid may be HCl or H₂SO₄. In some examples, the acid may be an aqueous acidic solution, for example, aqueous HCl or aqueous H₂SO₄. In some examples, the salt may be a halide salt, a cyanide salt or a thiolate salt. In some examples, the salt may be sodium chloride, lithium chloride, lithium bromide or potassium iodide. In some examples, the hydrolysis and decarboxylation may be a Krapcho decarboxylation reaction. In some examples, the base may be NaOH, KOH, Na₂CO₃, NaHCO₃, LiOH, Ba(OH)₂. In some examples, the base may be an aqueous basic solution, for example, aqueous NaOH or aqueous KOH.

In some examples, the hydrolysis and decarboxylation (which may comprise contacting the compound of Formula (V), the compound of Formula (VI) or the mixture thereof with an acid or a base in the presence of water) may comprise heating to a temperature of at least 20° C., for example, at least 25° C., at least 30° C., at least 35° C., at least 40° C., at least 45° C., at least 50° C., at least 55° C., at least 60° C., at least 65° C., at least 70° C., at least 75° C., at least 80° C., at least 85° C., at least 90° C., at least 95° C., or at least 100° C. In some examples, the hydrolysis and decarboxylation (which may comprise contacting the compound of Formula (V), the compound of Formula (VI) or the mixture thereof with an acid or a base in the presence of water) may comprise heating to a temperature of up to 100° C., for example, up to 95° C., up to 90° C., up to 85° C., up to 80° C., up to 75° C., up to 70° C., up to 65° C., up to 60° C., up to 55° C., up to 50° C., up to 45° C., up to 40° C., up to 35° C., up to 30° C., up to 25° C., or up to 20° C. In some examples, the hydrolysis and decarboxylation (which may comprise contacting the compound of Formula (V), the compound of Formula (VI) or the mixture thereof with an acid or a base in the presence of water) may comprise heating to a temperature of from 20° C. to 100° C., for example, 25° C. to 65° C., 30° C. to 70° C., 35° C. to 75° C., 40° C. to 80° C., 45° C. to 85° C., 50° C. to 90° C., 55° C. to 95° C., or 60° C. to 100° C.

In some examples, the method for making a compound of Formula (VIII), a compound of Formula (IX) or a mixture thereof may comprise contacting a compound of Formula (V), a compound of Formula (VI), or a mixture thereof with an acid. In some examples, the method for making a compound of Formula (VIII), a compound of Formula (IX) or a mixture thereof may comprise contacting a compound of Formula (V), a compound of Formula (VI), or a mixture thereof with a base. In some examples, the acid or base may be any acid or base capable of hydrolysing and decarboxylating the compound of Formula (V), the compound of Formula (VI) or the mixture thereof. In some examples, the base may be NaOH or KOH in the presence of water.

In some examples, the method for making a compound of Formula (VIII), a compound of Formula (IX) or a mixture thereof comprises a method for making a compound of Formula (VIII). In some examples, the method for making a compound of Formula (VIII), a compound of Formula (IX) or a mixture thereof comprises a method for making a compound of Formula (IX). In some examples, the method for making a compound of Formula (VIII), a compound of Formula (IX) or a mixture thereof comprises a method for making a mixture of a compound of Formula (VIII) and a compound of Formula (IX).

In some examples, the method for making a compound of Formula (VIII), a compound of Formula (IX) or a mixture thereof comprises hydrolysis and decarboxylation of the product of a method for making a compound of Formula (V), a compound of Formula (VI) or a mixture thereof, for example, by contacting the product of a method for making a compound of Formula (V), a compound of Formula (VI) or a mixture thereof with an acid or a base in the presence of water. In some examples, the method for making a compound of Formula (VIII), a compound of Formula (IX) or a mixture thereof comprises making a compound of Formula (V), a compound of Formula (VI) or a mixture thereof by a method described herein; and hydrolysing and decarboxylating the compound of Formula (V), the compound of Formula (VI) or the mixture thereof, for example by contacting the compound of Formula (V), the compound of Formula (VI) or the mixture thereof with an acid or a base in the presence of water (for example, as summarised in the schematic below).

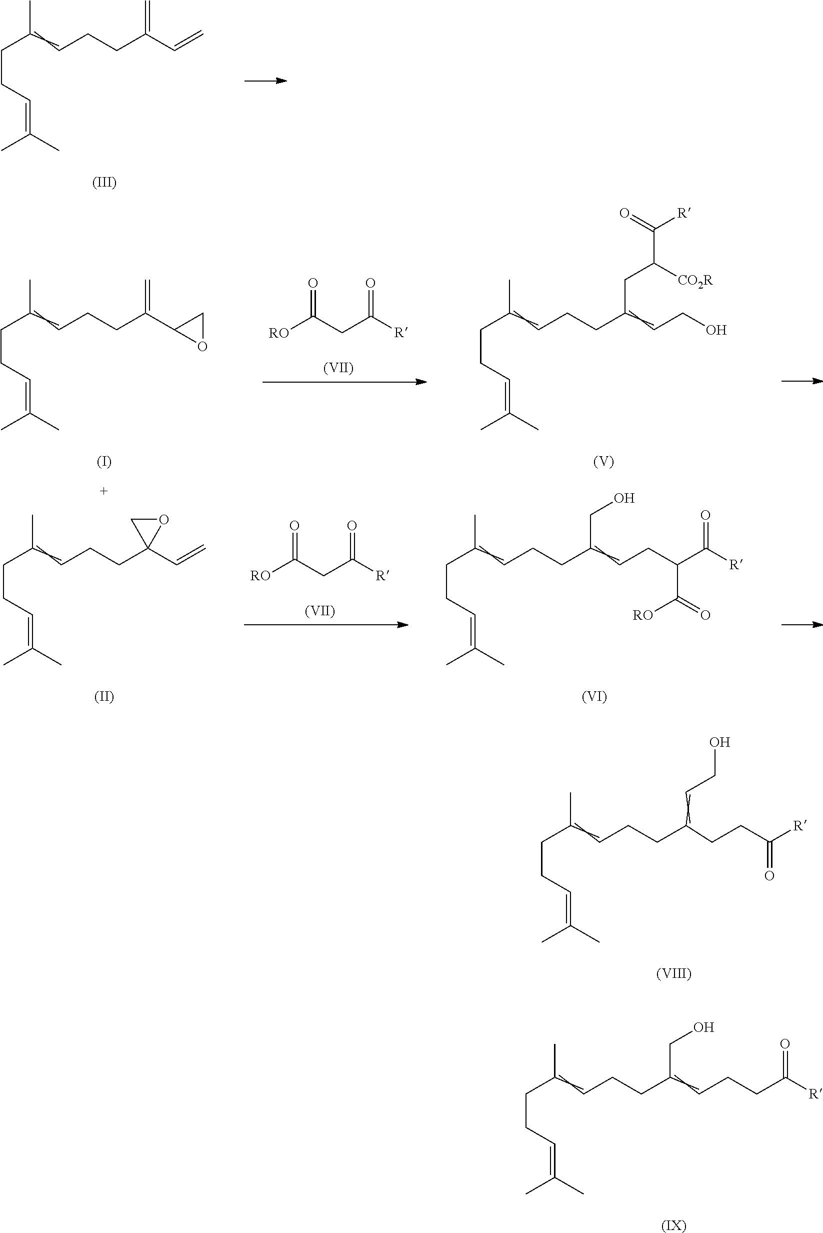

In some examples, the method for making a mixture of a compound of Formula (VIII) and a compound of Formula (IX) comprises hydrolysis and decarboxylation of the product of a method for making a mixture of a compound of Formula (V) and a compound of Formula (VI), for example, by contacting the product of a method for making a mixture of a compound of Formula (V) and a compound of Formula (VI) with an acid or a base. In some examples, the method for making a mixture of a compound of Formula (VIII) and a compound of Formula (IX) comprises making a mixture of a compound of Formula (V) and a compound of Formula (VI) by a method described herein; and hydrolysing and decarboxylating the mixture of the compound of Formula (V) and the compound of Formula (VI), for example, by contacting the mixture of the compound of Formula (V) and the compound of Formula (VI) with an acid or a base in the presence of water (for example, as summarised in the schematic below).

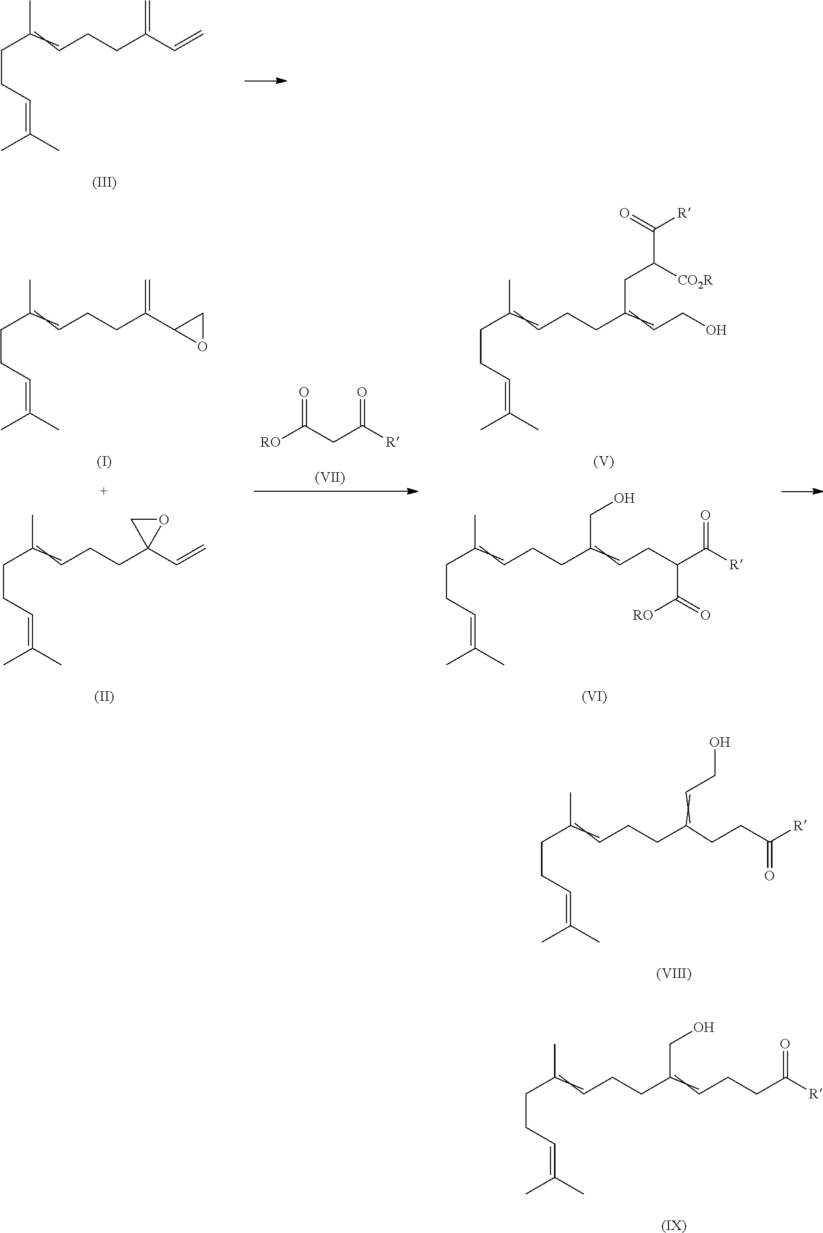

In some examples, the method for making a mixture of a compound of Formula (VIII) and a compound of Formula (IX) comprises making a mixture of a compound of Formula (I) and a compound of Formula (II) by a method described herein, said mixture further comprising unreacted compound of Formula (III), followed by contacting said mixture with a compound of Formula (VII) and a palladium catalyst resulting in a mixture comprising a compound of Formula (V), a compound of Formula (VI) and unreacted compound of Formula (III). In some examples, the method for making a mixture of a compound of Formula (VIII) and a compound of Formula (IX) comprises making a mixture of a compound of Formula (V) and a compound of Formula (VI) starting from a mixture comprising a compound of Formula (I), a compound o Formula (II), and optionally a compound of Formula (III) in a one-pot procedure.

In some examples, the method for making a compound of Formula (VIII) may comprise making a mixture of a compound of Formula (VIII) and a compound of Formula (IX) and separating the compound of Formula (VIII) from the mixture, for example, by flash chromatography or distillation. In some examples, the method for making a compound of Formula (IX) may comprise making a mixture of a compound of Formula (VIII) and a compound of Formula (IX) and separating the compound of Formula (IX) from the mixture, for example, by flash chromatography or distillation.

In some examples, the method produces a mixture of E,E- and E,Z-Formula (VIII) and/or a mixture of E,E- and E,Z-Formula (IX).

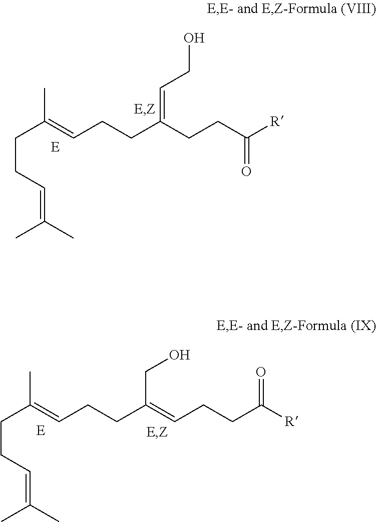

The method may produce a mixture of the E,E- and E,Z-isomers and further comprise separating the E,E- and the E,Z-isomers, for example, by flash chromatography or distillation. In some examples, the mixture of isomers produced by the method corresponds to the mixture of isomers of the compound of Formula (V), the compound of Formula (VI) or the mixture thereof used as the reagent, that is, the method does not change the isomer mixture.

In some examples, the method for making a compound of Formula (IX) comprises hydrolysis and decarboxylation of the product of a method for making a compound of Formula (VI), for example, by contacting the product of a method for making a compound of Formula (VI) with an acid or a base in the presence of water. In some examples, the method for making a compound of Formula (IX) comprises making a compound of

Formula (VI) by a method described herein; and hydrolysing and decarboxylating the compound of Formula (VI), for example, by contacting the compound of Formula (VI) with an acid or a base in the presence of water.

In some examples, the method for making a compound of Formula (IX) comprises making a mixture of the compound of Formula (V) and the compound of Formula (VI) by a method described herein; separating the compound of Formula (VI) from the mixture; and hydrolysing and decarboxylating the compound of Formula (VI), for example, by contacting the compound of Formula (VI) with an acid or a base in the presence of water.

In some examples, the method for making a compound of Formula (IX) comprises making a mixture of a compound of Formula (I) and a compound of Formula (II) by a method described herein; separating the compound of Formula (II) from the mixture, for example, by a method described herein; making a compound of Formula (VI) from the compound of Formula (II) by a method described herein; and making a compound of Formula (IX) from the compound of Formula (VI) by a method described herein.

In some examples, the method for making a compound of Formula (VIII), a compound of Formula (IX) or a mixture thereof may comprise contacting a compound of Formula (V), a compound of Formula (VI) or a mixture thereof with an aqueous acid or an aqueous base for at least 30 min, for example, at least 1 h, at least 2 h, at least 3 h, at least 4 h, at least 5 h, at least 6 h, at least 7 h, at least 8 h, at least 9 h, at least 10 h, at least 11 h, at least 12 h. In some examples, the method for making a compound of Formula (VIII), a compound of Formula (IX) or a mixture thereof may comprise contacting a compound of Formula (V), a compound of Formula (VI) or a mixture thereof with an aqueous acid or an aqueous base for up to 12 h, for example, up to 11 h, up to 10 h, up to 9 h, up to 8 h, up to 7 h, up to 6 h, up to 5 h, up to 4 h, up to 3 h, up to 2 h, up to 1 h, up to 30 min. In some examples, the method for making a compound of Formula (VIII), a compound of Formula (IX) or a mixture thereof may comprise contacting a compound of Formula (V), a compound of Formula (VI) or a mixture thereof with an aqueous acid or an aqueous base for from about 30 min to about 12 h, for example, about 1 h to about 11 h, about 2 h to about 10 h, about 3 h to about 9 h, about 4 h to about 8 h, about 5 h to about 7 h, about 6 h to about 12.

Method for making compounds of Formulae (VIII) and (IX) (R′=Me) from compounds of Formulae (I) and (II) and acetone

In another aspect, there is provided a method for making a compound of Formula (VIII), a compound of Formula (IX) or a mixture thereof,

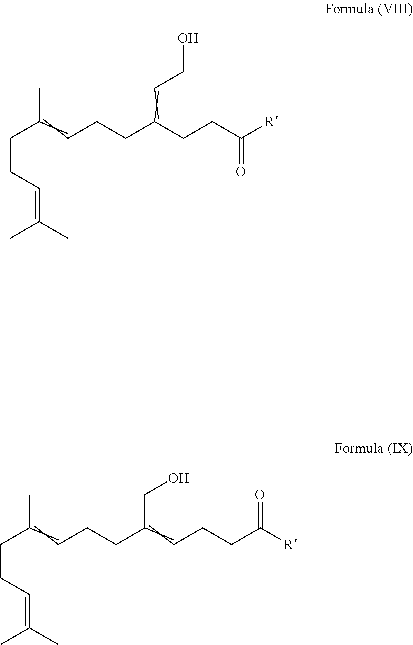

wherein R′ is methyl and wherein the method comprises contacting a compound of Formula (I), a compound of Formula (II) or a mixture thereof with acetone and a palladium catalyst.

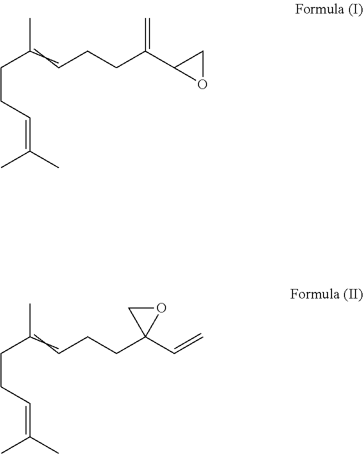 [SMILES: C=C(CCC=C(C)CCC=C(C)C)C1CO1 ; C=CC1(CCC=C(C)CCC=C(C)C)CO1]

In some examples, the stereochemistry of the double bond (i.e., the double bond between C6 and C7 of the compound of Formula (III)) remains unchanged during the method of making a compound of Formula (VIII), a compound of Formula (IX) or a mixture thereof.

The palladium catalyst may be any palladium catalyst described for the method for making compounds of Formulae (V) and (VI).

In some examples, the method for making a compound of Formula (VIII), a compound of Formula (IX) or a mixture thereof, wherein R′ is methyl, comprises contacting a compound of Formula (I), a compound of Formula (II) or a mixture thereof with acetone and a palladium catalyst at room temperature or above, for example, a temperature of up to 50° C., up to 40° C. In some examples, the method for making a compound of Formula (VIII), a compound of Formula (IX) or a mixture thereof, wherein R′ is methyl, comprises contacting a compound of Formula (I), a compound of Formula (II) or a mixture thereof with acetone and a palladium catalyst at temperature of from 20° C. to 50° C., for example, 25° C. to 45° C., 30° C. to 40° C., or 40° C. to 45° C.

In some examples, the method for making a compound of Formula (VIII), a compound of Formula (IX) or a mixture thereof, wherein R′ is methyl, comprises contacting a compound of Formula (I), a compound of Formula (II) or a mixture thereof with acetone and a palladium catalyst for at least 12 h, for example, at least 18 h, at least 24 h, at least 30 h, at least 36 h, at least 42 h, at least 48 h, at least 54 h, at least 60 h, at least 66 h, at least 72 h. In some examples, the method for making a compound of Formula (VIII), a compound of Formula (IX) or a mixture thereof, wherein R′ is methyl, comprises contacting a compound of Formula (I), a compound of Formula (II) or a mixture thereof with acetone and a palladium catalyst for up to 72 h, for example, up to 66 h, up to 60 h, up to 54 h, up to 48 h, up to 42 h, up to 36 h, up to 30 h, up to 24 h, up to 18 h, or up to 12 h. In some examples, the method for making a compound of Formula (VIII), a compound of Formula (IX) or a mixture thereof, wherein R′ is methyl, comprises contacting a compound of Formula (I), a compound of Formula (II) or a mixture thereof with acetone and a palladium catalyst for from about 12 h to about 72 h, for example, 18 h to 66 h, 24 h to 60 h, 30 h to 54 h, 36 h to 48 h, or 36 h to 42 h.

In some examples, the method for making a compound of Formula (VIII), a compound of Formula (IX) or a mixture thereof, wherein R′is methyl, comprises a method for making a compound of Formula (VIII). In some examples, the method for making a compound of Formula (VIII), a compound of Formula (IX) or a mixture thereof, wherein R′ is methyl, comprises a method for making a compound of Formula (IX). In some examples, the method for making a compound of Formula (VIII), a compound of Formula (IX) or a mixture thereof, wherein R′ is methyl, comprises a method for making a mixture of a compound of Formula (VIII) and a compound of Formula (IX).

In some examples, the method for making a compound of Formula (VIII), a compound of Formula (IX) or a mixture thereof, wherein R′ is methyl, comprises contacting the product of a method for making a compound of Formula (I), a compound of Formula (II) or a mixture thereof with acetone and a palladium catalyst. In some examples, the method for making a compound of Formula (VIII), a compound of Formula (IX) or a mixture thereof comprises making a compound of Formula (I), a compound of Formula (II) or a mixture thereof by a method described herein (in some examples, unreacted compound of Formula (III) remains part of the product mixture); and contacting the compound of Formula (I), the compound of Formula (II) or the mixture thereof with acetone and a palladium catalyst (for example, as summarised in the schematic below).

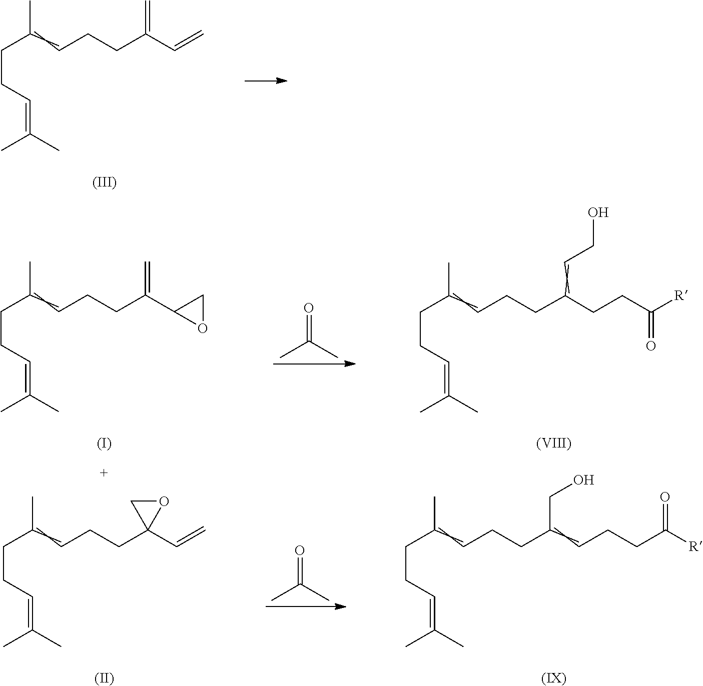

In some examples, the method for making a mixture of a compound of Formula (VIII) and a compound of Formula (IX), wherein R′is methyl, comprises contacting the product of a method for making a mixture of a compound of Formula (I) and a compound of Formula (II) with acetone and a palladium catalyst. In some examples, the method for making a mixture of a compound of Formula (VIII) and a compound of Formula (IX), wherein R′ is methyl, comprises making a mixture of a compound of Formula (I) and a compound of Formula (II) by a method described herein (in some examples, unreacted compound of Formula (III) remains part of the product mixture); and contacting the mixture of the compound of Formula (I) and the compound of Formula (II) with acetone and a palladium catalyst.

In some examples, the method for making a compound of Formula (IX) comprises making a mixture of a compound of Formula (VIII) and a compound of Formula (IX) and separating the compound of Formula (IX) from the mixture.

In some examples, the method for making a compound of Formula (IX), wherein R′ is methyl, comprises contacting the product of a method for making a compound of Formula (II) with acetone and a palladium catalyst. In some examples, the method for making a compound of Formula (IX), wherein R′ is methyl, comprises making a compound of Formula (II) by a method described herein; and contacting the compound of Formula (II) with acetone and a palladium catalyst.

In some examples, the method for making a compound of Formula (IX) comprises making a mixture of a compound of Formula (I) and a compound of Formula (II); separating the compound of Formula (II) from the mixture; and contacting the compound of Formula (II) with acetone and a palladium catalyst.

Method for Making a Compound of Formula (X), a Compound of Formula (XII) or a Mixture Thereof

In a further aspect, there is provided a method for making a compound of Formula (X), a compound of Formula (XII) or a mixture thereof,

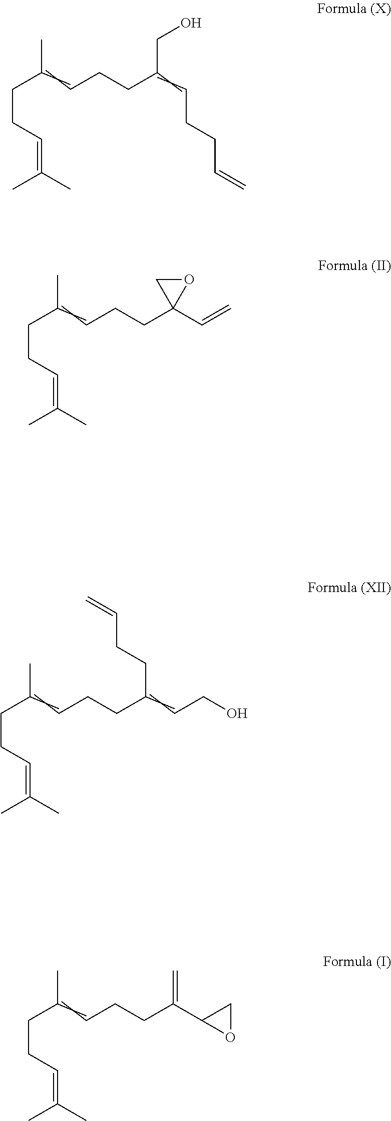 [SMILES: C=CCCC=C(CO)CCC=C(C)CCC=C(C)C ; C=CC1(CCC=C(C)CCC=C(C)C)CO1 ; C=CCCC(=CCO)CCC=C(C)CCC=C(C)C ; C=C(CCC=C(C)CCC=C(C)C)C1CO1]

wherein the method comprises a copper-catalysed alkylation of a compound of Formula (II), a compound of Formula (I) or a mixture thereof.

In a further aspect, there is provided a method for making a compound of Formula (X)

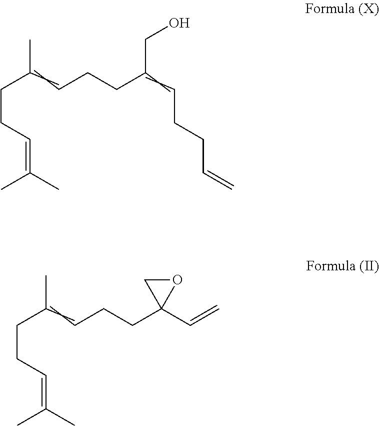 [SMILES: C=CCCC=C(CO)CCC=C(C)CCC=C(C)C ; C=CC1(CCC=C(C)CCC=C(C)C)CO1]

wherein the method comprises a copper-catalysed alkylation of a compound of Formula (II)

In some examples, the stereochemistry of the double bond (i.e., the double bond between C6 and C7 of the compound of Formula (III)) remains unchanged during the process.

In some examples, the copper-catalysed alkylation of a compound of Formula (II), a compound of Formula (I) or a mixture thereof may comprise contacting the compound of Formula (II), the compound of Formula (I), or the mixture thereof with a Grignard reagent. In some examples, the copper-catalysed alkylation of a compound of Formula (II) may comprise contacting the compound of Formula (II) with a Grignard reagent. In some examples, the Grignard reagent may comprise an allyl group. In some examples, the

Grignard reagent may be an allyl magnesium halide. In some examples, the copper-catalysed alkylation of a compound of Formula (II), a compound of Formula (I) or a mixture thereof may comprise contacting a compound of Formula (II), a compound of Formula (I) or a mixture thereof with an allyl magnesium halide. In some examples, the copper-catalysed alkylation of a compound of Formula (II) may comprise contacting a compound of Formula (II) with an allyl magnesium halide.

In some examples, the allyl magnesium halide is selected from allyl magnesium chloride and allyl magnesium bromide. In some examples, the allyl magnesium halide is allyl magnesium chloride.

In some examples, the method for making a compound of Formula (X), a compound of Formula (XII), or a mixture thereof comprises contacting a compound of Formula (II), a compound of Formula (I) or a mixture thereof with a Grignard reagent and a copper catalyst. In some examples, the method for making a compound of Formula (X), a compound of Formula (XII), or a mixture thereof comprises contacting a compound of Formula (II), a compound of Formula (I), or a mixture thereof with an allyl magnesium halide and a copper catalyst. In some examples, the method for making a compound of Formula (X) comprises contacting a compound of Formula (II) with a Grignard reagent and a copper catalyst. In some examples, the method for making a compound of Formula (X) comprises contacting a compound of Formula (II) with an allyl magnesium halide and a copper catalyst. In some examples, the copper catalyst is selected from Cul, CuBr, CuCl or Li₂CuCl₄. In some examples, the copper catalyst is a combination of a Lewis base with one or more of Cul, CuBr or CuCl. In some examples, the copper catalyst is CuBr·SMe₂.

In some examples, the method for making a compound of Formula (X), a compound of Formula (XII) or a mixture thereof produces a mixture of E,E- and E,Z-Formula (X) and/or a mixture of E,E- and E,Z-Formula (XII). In some examples, the method for making a compound of Formula (X), a compound of Formula (XII), or a mixture thereof produces a mixture of E,E- and E,Z-Formula (X) and/or a mixture of E,E- and E,Z-Formula (XII) and the method further comprises separating the E,E- and the E,Z-isomers. In some examples, the method for making a compound of Formula (X) produces a mixture of E,E- and E,Z-Formula (X). In some examples, the method for making a compound of Formula (X) produces a mixture of E,E- and E,Z-Formula (X) and the method further comprises separating the E,E- and the E,Z-isomers.

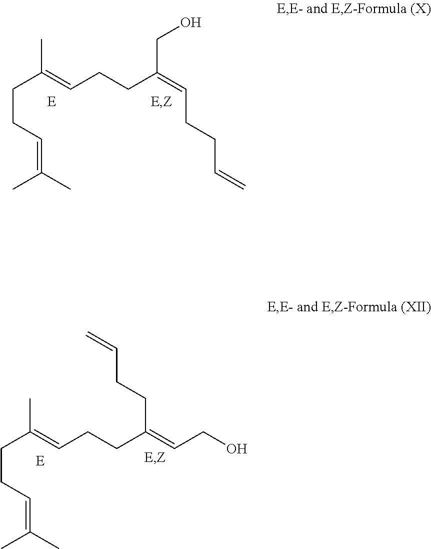 [SMILES: C=CCC/C=C(/CO)CC/C=C(\C)CCC=C(C)C ; C=CCC/C(=C\CO)CC/C=C(\C)CCC=C(C)C]

In some examples, the mixture of E,Z- and E,E-Formula (X) is a 10:1 to 1:10 mixture, for example, a 9:1 to 1:1 mixture, an 8:1 to 2:1 mixture, a 7:1 to 4:1 mixture, a 6:1 to 5:1 mixture. In some examples, the mixture of E,Z- and E,E-Formula (X) is a 6:1 mixture. In some examples, the major isomer produced by the method for making a compound of Formula (X) is E,Z-Formula (X).

In some examples, the method for making a compound of Formula (X) produces a mixture of E,E- and E,Z-Formula (X) and further comprises separating the E,E- and E,Z-isomers. In some examples, the method for making a compound of Formula (XII) produces a mixture of E,E- and E,Z-Formula (XII) and further comprises separating the E,E- and E,Z-isomers. In some examples, the method for making a mixture of a compound of Formula (X) and a compound of Formula (XII) produces a mixture of E,E- and E,Z-Formula (X) and a mixture of E,E- and E,Z-Formula (XII) and further comprises separating the compound of Formula (X) from the compound of Formula (XII) and/or separating the E,E- and E,Z-isomers.

In some examples, the method for making a compound of Formula (X), a compound of Formula (XII), or a mixture thereof comprises the copper-catalysed alkylation of the product of a method for making a compound of Formula (I), a compound of Formula (II), or a mixture thereof, for example, by contacting the product of a method for making a compound of Formula (I), a compound of Formula (II) or a mixture thereof with a Grignard reagent. In some examples, the method for making a compound of Formula (X), a compound of Formula (XII), or a mixture thereof comprises making a compound of Formula (I), a compound of Formula (II) or a mixture thereof by a method described herein; and performing a copper-catalysed alkylation of the compound of Formula (I), the compound of Formula (II), or the mixture thereof (for example, as summarised in the schematic below).

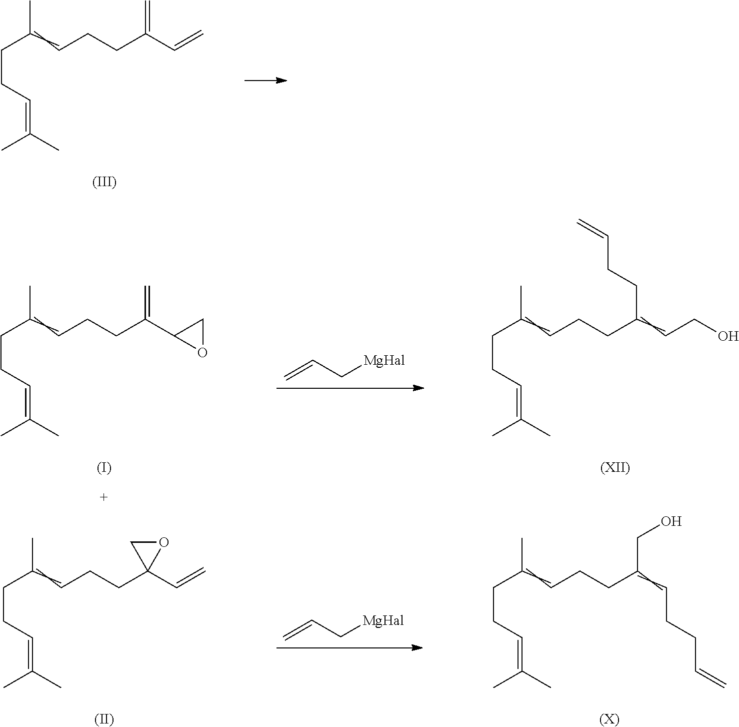

In some examples, the method for making a compound of Formula (X) comprises the copper-catalysed alkylation of the product of a method for making a compound of Formula (II), for example, by contacting the product of a method for making a compound of Formula (II) with a Grignard reagent, such as an allyl magnesium halide. In some examples, the method for making a compound of Formula (X) comprises making a compound of Formula (II) by a method described herein; and performing a copper-catalysed alkylation of the compound of Formula (II), for example, by contacting the compound of Formula (II) with a Grignard reagent, such as an allyl magnesium halide.

In some examples, the method for making a compound of Formula (X) comprises making a mixture of a compound of Formula (I) and a compound of Formula (II); separating the compound of Formula (II) from the mixture; and performing a copper-catalysed alkylation of the compound of Formula (II), for example, by contacting the compound of Formula (II) with a Grignard reagent, such as an allyl magnesium halide.

In some examples, the method for making a compound of Formula (X), a compound of Formula (XII), or a mixture thereof comprises contacting a compound of Formula (II), a compound of Formula (I), or a mixture thereof with an allyl magnesium halide under cooling, for example, at a temperature of room temperature or below. In some examples, the cooling is to a temperature of −30° C. or above, for example, −25° C. or above, −20° C. or above, −15° C. or above, −10° C. or above, −5° C. or above, 0° C. or above, 5° C. or above, 10° C. or above, 15° C. or above, 20° C. or above. In some examples, the cooling is to a temperature of from −30° C. to 20° C., for example, −30° C. to 0° C., −30° C. to −10° C., -25° C. to −20° C. In some examples, the method for making a compound of Formula (X), a compound of Formula (XII), or a mixture thereof comprises contacting a compound of Formula (II), a compound of Formula (I), or a mixture thereof with an allyl magnesium halide for at least 30 min, for example, at least 1 h, at least 2 h, at least 3 h, at least 4 h, at least 5 h, at least 6 h, at least 7 h, at least 8 h, at least 9 h, at least 10 h. In some examples, the method for making a compound of Formula (X), a compound of Formula (XII), or a mixture thereof comprises contacting a compound of Formula (II), a compound of Formula (I), or a mixture thereof with an allyl magnesium halide for up to 10 h, for example, up to 9 h, up to 8 h, up to 7 h, up to 6 h, up to 5 h, up to 4 h, up to 3 h, up to 2 h, up to 1 h, up to 30 min. In some examples, the method for making a compound of Formula (X), a compound of Formula (XII), or a mixture thereof comprises contacting a compound of Formula (II), a compound of Formula (I), or a mixture thereof with an allyl magnesium halide for from about 0.5 h to about 10 h, for example, 1 h to 9 h, 2 h to 8 h, 3 h to 7 h, 4 h to 6 h, 5 h to 19 h.

Method for making a compound of Formula (IX) and/or a compound of Formula (VIII) from a compound of Formula (X) and/or a compound of Formula (XII)

In another aspect, there is provided a method for making a compound of Formula (IX), a compound of Formula (VIII) or a mixture thereof, wherein R′ is methyl, the method comprising a palladium-catalysed Wacker oxidation of a compound of Formula (X), a compound of Formula (XII) or a mixture thereof. In some examples, the palladium-catalysed Wacker oxidation of a compound of Formula (X), a compound of Formula (XII) or a mixture thereof comprises contacting a compound of Formula (X), a compound of Formula (XII) or a mixture thereof with a palladium catalyst (e.g., palladium chloride) and gaseous oxygen. In some examples, the method for making a compound of Formula (IX), a compound of Formula (VIII) or a mixture thereof , wherein R′is methyl, comprises contacting a compound of Formula (X), a compound of Formula (XII) or a mixture thereof with a palladium catalyst (e.g., palladium chloride) and gaseous oxygen.

In some examples, the E/Z-ratio of the double bonds remains unchanged during the process. In some examples, the reagent comprises a mixture of stereoisomers and the isomeric ratio remains unchanged during the reaction. In some examples, the reagent comprises a single stereoisomer.

In another aspect, there is provided a method for making a compound of Formula (IX), wherein R′ is methyl, the method comprising a palladium-catalysed Wacker oxidation of a compound of Formula (X). In some examples, the palladium-catalysed Wacker oxidation of a compound of Formula (X) comprises contacting a compound of Formula (X) with a palladium catalyst (e.g. palladium chloride) and gaseous oxygen. In some examples, the method for making a compound of Formula (IX), wherein R′ is methyl, comprises contacting a compound of Formula (X) with a palladium catalyst (e.g. palladium chloride) and gaseous oxygen.

In some examples, the palladium catalyst is selected from palladium chloride, Pd(OAc)₂, Pd(TFA)₂, Pd(PhCN)Cl₂, Pd(MeCN)Cl₂, or supported Pd (P/C, Pd/ZrO₂).

In some examples, the method for making a compound of Formula (IX), a compound of Formula (VIII), or a mixture thereof, wherein R′ is methyl, comprises contacting a compound of Formula (X), a compound of Formula (XII), or a mixture thereof with a palladium catalyst (e.g. palladium chloride), gaseous oxygen and a copper catalyst (e.g. copper chloride). In some examples, the method for making a compound of Formula (IX), wherein R′ is methyl comprises contacting a compound of Formula (X) with a palladium catalyst (e.g. palladium chloride), gaseous oxygen and a copper catalyst (e.g., copper chloride). In some examples, the copper catalyst may be selected from CuCl, CuCl₂, and Cu(OAc)₂.

In some examples, the method for making a compound of Formula (IX), a compound of Formula (VIII), or a mixture thereof, wherein R′ is methyl, comprises performing a palladium-catalysed Wacker oxidation of the product of a method for making a compound of Formula (X), a compound of Formula (XII), or a mixture thereof, for example, by contacting the product of a method for making a compound of Formula (X), a compound of Formula (XII), or a mixture thereof with a palladium catalyst (e.g., palladium chloride) and gaseous oxygen. In some examples, the method for making a compound of Formula (IX), a compound of Formula (VIII), or a mixture thereof, wherein R′ is methyl, comprises making a compound of Formula (X), a compound of Formula (XII), or a mixture thereof by a method described herein; and performing a palladium-catalysed Wacker oxidation of the compound of Formula (X), the compound of Formula (XII), or the mixture thereof, for example, by contacting the compound of Formula (X), the compound of Formula (XII), or the mixture thereof with a palladium catalyst (e.g., palladium chloride) and gaseous oxygen (for example, as summarised in the schematic below).

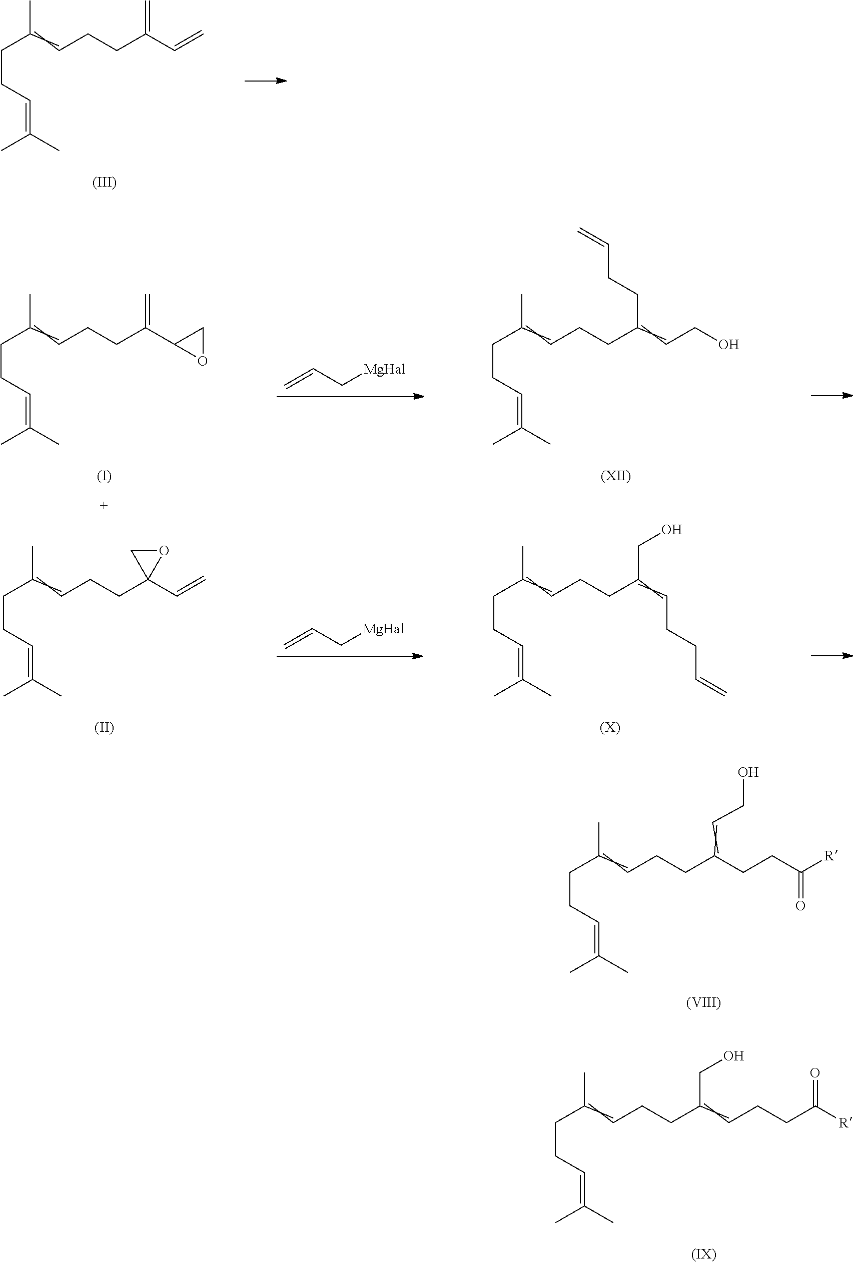

In some examples, the method for making a compound of Formula (IX), wherein R′ is methyl, comprises performing a palladium-catalysed Wacker oxidation of the product of a method for making a compound of Formula (X), for example, by contacting the product of a method for making a compound of Formula (X) with palladium chloride and gaseous oxygen. In some examples, the method for making a compound of Formula (IX), wherein R′ is methyl, comprises making a compound of Formula (X) by a method described herein; and performing a palladium-catalysed Wacker oxidation of the compound of Formula (X), for example, by contacting the compound of Formula (X) with palladium chloride and gaseous oxygen.

In some examples, the method for making a compound of Formula (IX), wherein R′ is methyl, comprises making a compound of Formula (II) by a method described herein; making a compound of Formula (X) from the compound of Formula (II) by a method described herein; and performing a palladium-catalysed Wacker oxidation of the compound of Formula (X), for example, by contacting the compound of Formula (X) with palladium chloride and gaseous oxygen.

In some examples, the method for making a compound of Formula (IX), wherein R′ is methyl, comprises making a mixture of a compound of Formula (I) and a compound of Formula (II) by a method described herein; separating the compound of Formula (II) from the mixture, for example, by a method described herein; making a compound of Formula (X) from the compound of Formula (II) by a method described herein; and performing a palladium-catalysed Wacker oxidation of the compound of Formula (X), for example, by contacting the compound of Formula (X) with palladium chloride and gaseous oxygen (for example, as summarised in the schematic below).

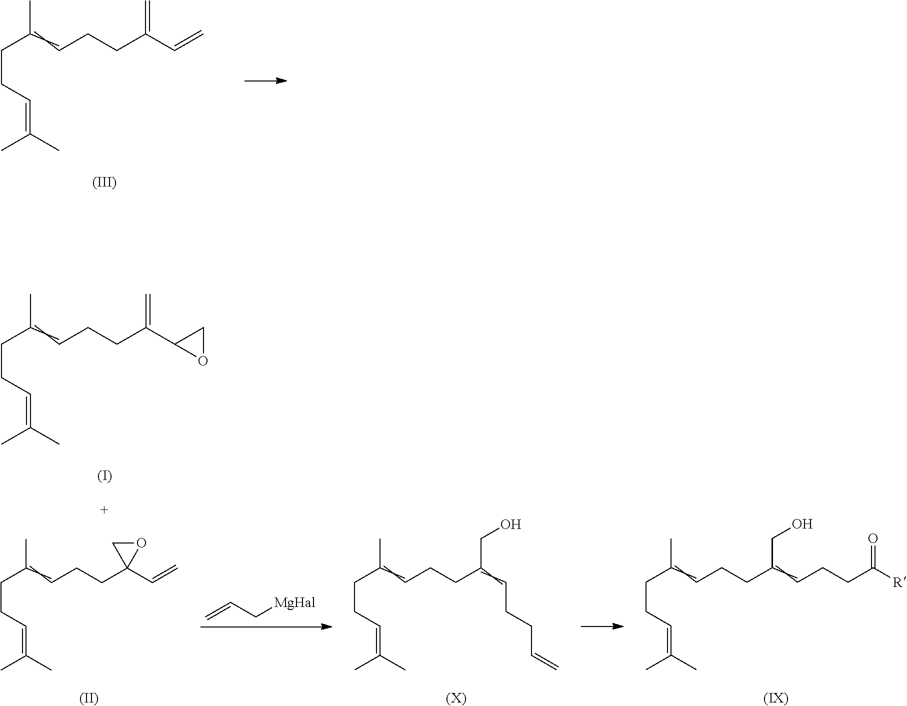

In some examples, the method for making a compound of Formula (IX), a compound of Formula (VIII) or a mixture thereof, wherein R′ is methyl, comprises a method for making a compound of Formula (IX). In some examples, the method for making a compound of Formula (IX), a compound of Formula (VIII) or a mixture thereof, wherein R′ is methyl, comprises a method for making a compound of Formula (VIII). In some examples, the method for making a compound of Formula (IX), a compound of Formula (VIII) or a mixture thereof, wherein R′ is methyl, comprises a method for making a mixture of a compound of Formula (IX) and a compound of Formula (VIII).

In some examples, the method for making a compound of Formula (IX), a compound of Formula (VIII) or a mixture thereof, wherein R′ is methyl, comprises a method for making a mixture of E,E- and E,Z-Formula (IX) and/or a mixture of E,E- and E,Z-Formula (VIII). In some examples, the method further comprises separating the E,E- and E,Z-isomers. In some examples, the ratio of the mixture of isomers produced by the method corresponds to the ratio of the mixture of isomers of the reagent used in the method.

In some examples, the method for making a compound of Formula (IX), wherein R′ is methyl, comprises a method for making a mixture of E,E- and E,Z-Formula (IX). In some examples, the method for making a compound of Formula (IX), wherein R′ is methyl, comprises a method for making E,E-Formula (IX). In some examples, the method for making E,E-Formula (IX) comprises making a mixture of E,E- and E,Z-Formula (IX) and separating E,E-Formula (IX) from the mixture. In some examples, the method for making a compound of Formula (IX), wherein R′ is methyl, comprises a method for making E,Z-Formula (IX). In some examples, the method for making E,Z-Formula (IX) comprises making a mixture of E,E- and E,Z-Formula (IX) and separating E,Z-Formula (IX) from the mixture.

In some examples, the method for making E,E-Formula (IX) comprises contacting the product of a method for making E,E-Formula (X) with palladium chloride and gaseous oxygen. In some examples, the method for making E,E-Formula (IX) comprises making E,E-Formula (X) by a method described herein; and contacting E,E-Formula (X) with palladium chloride and gaseous oxygen. In some examples, the method for making E,E-Formula (IX) comprises making a mixture of E,E- and E,Z-Formula (X) by a method described herein; separating E,E-Formula (X) from the mixture; and contacting E,E-Formula (X) with palladium chloride and gaseous oxygen.

In some examples, the method for making E,Z-Formula (IX) comprises contacting the product of a method for making E,Z-Formula (X) with palladium chloride and gaseous oxygen. In some examples, the method for making E,Z-Formula (IX) comprises making E,Z-Formula (X) by a method described herein; and contacting E,Z-Formula (X) with palladium chloride and gaseous oxygen. In some examples, the method for making E,Z-Formula (IX) comprises making a mixture of E,E- and E,Z-Formula (X) by a method described herein; separating E,Z-Formula (X) from the mixture; and contacting E,Z-Formula (X) with palladium chloride and gaseous oxygen.

The following numbered paragraphs define additional aspects of the present disclosure:
1. A method for making a compound of Formula (VIII), a compound of Formula (IX) or a mixture thereof,

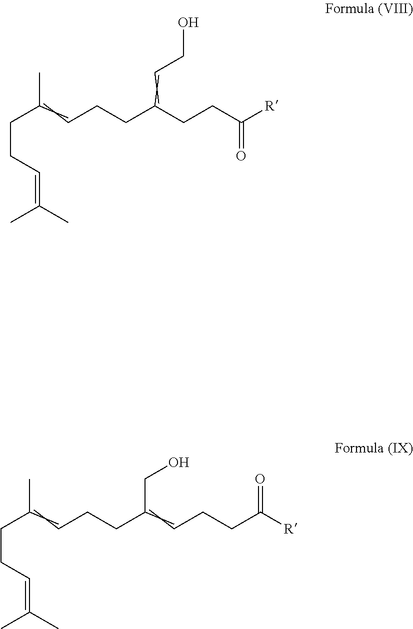

wherein the method comprises hydrolysis and decarboxylation of a compound of Formula (V), a compound of Formula (VI), or a mixture thereof,

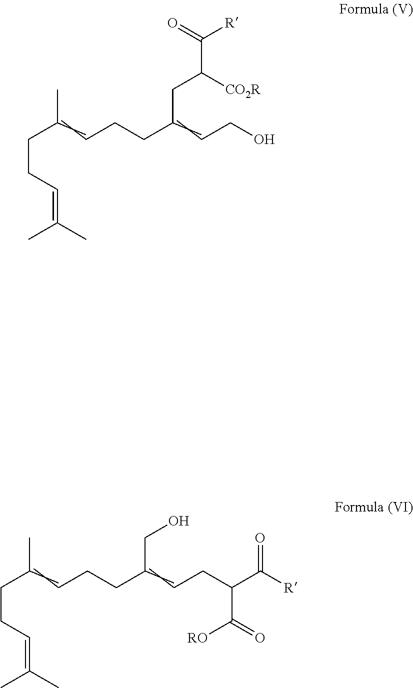

wherein R′ is selected from the group consisting of a C1 to C6 alkyl group and an OR group, and wherein R is selected from the group consisting of hydrogen or a C1 to C6 alkyl group.
2. The method of paragraph 1, wherein hydrolysis and decarboxylation of a compound of Formula (V), a compound of Formula (VI), or a mixture thereof comprises contacting the compound of formula (V), the compound of Formula (VI), or the mixture thereof with an acid, a base, optionally in the presence of water, or a nucleophile.
3. The method of any of paragraphs 1 or 2, wherein R′ is methyl.

4. The method of any of paragraphs 1 to 3, wherein the method comprises contacting a compound of Formula (V), a compound of Formula (VI), or a mixture thereof with a base, for example, NaOH or KOH.
5. A method for making a compound of Formula (VIII), a compound of Formula (IX) or a mixture thereof, wherein R′ is methyl,

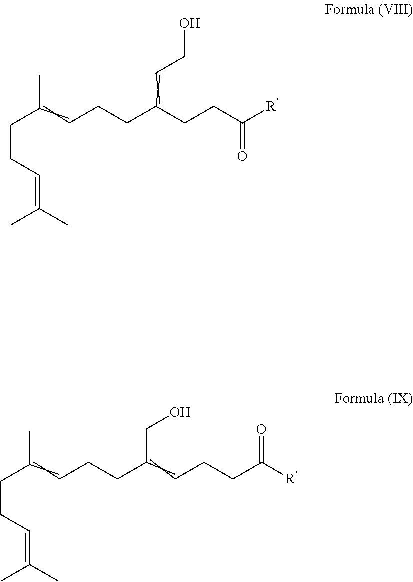

wherein the method comprises contacting a compound of Formula (I), a compound of Formula (II) or a mixture thereof with acetone and a palladium catalyst.

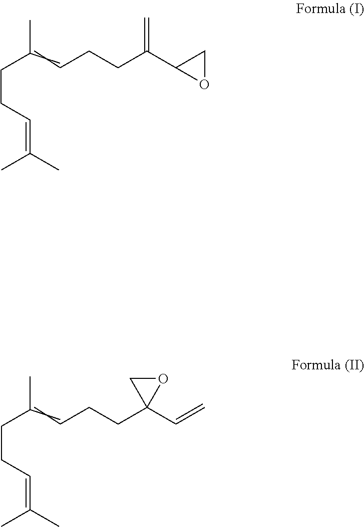 [SMILES: C=C(CCC=C(C)CCC=C(C)C)C1CO1 ; C=CC1(CCC=C(C)CCC=C(C)C)CO1]

6. The method of paragraph 5, wherein the palladium catalyst is selected from a Pd(0) catalyst, such as Pd(PPh₃)₄, Pd₂(dba)₃ or solvated Pd₂(dba)₃ (such as Pd₂(dba)₃·dba or Pd₂(dba)₃·CHCl₃); a Pd(II) catalyst, such as Pd(OAc)₂, Pd(acac)₂, Pd(MeCN)₂Cl₂, Pd(COD)Cl₂, [(C₃H₅)PdCl]₂, or PdCl₂; or a supported heterogeneous Pd catalyst, such as Pd/C.
7. A method for making a compound of Formula (IX), a compound of Formula (VIII), or a mixture thereof, wherein R′ is methyl,

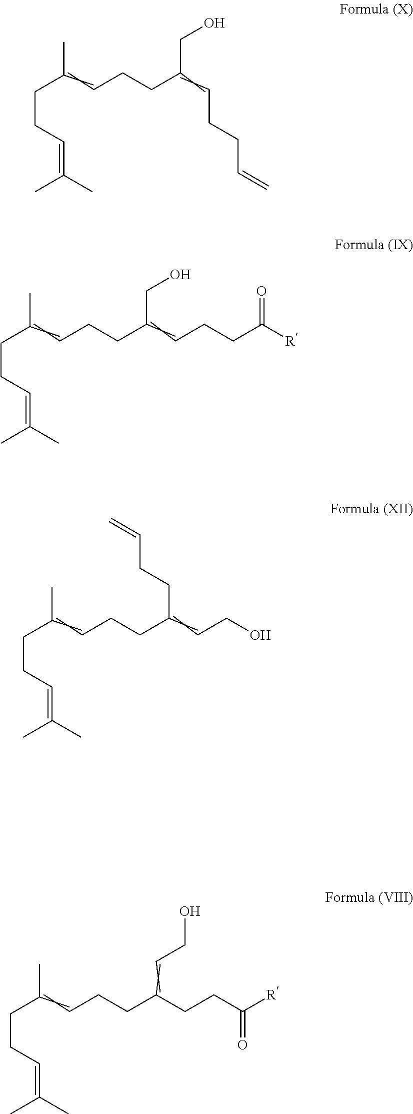

the method comprising a palladium-catalysed Wacker oxidation of a compound of Formula (X), a compound of Formula (XII), or a mixture thereof.
8. The method of paragraph 7, wherein the method comprises contacting the compound of Formula (X), the compound of Formula (XII), or the mixture thereof with a palladium catalyst (such as palladium (II) chloride) and gaseous oxygen.
9. The method of any of paragraphs 7 or 8, wherein contacting a compound of Formula (X), a compound of Formula (XII), or a mixture thereof with a palladium catalyst (such as palladium (II) chloride) and gaseous oxygen comprises contacting a compound of Formula (X), a compound of Formula (XII), or a mixture thereof with palladium chloride, gaseous oxygen and a copper catalyst (such as copper chloride).

10. The method of any of paragraphs 7 to 9, wherein the compound of Formula (X) is a mixture of E,E- and E,Z-Formula (X) and the method further comprises separating E,E- and E,Z-Formula (IX); and/or wherein the compound of Formula (XII) is a mixture of E,E- and E,Z-Formula (XII) and the method further comprises separating E,E- and E,Z-Formula (VIII).
11. A method for making a compound of Formula (V), a compound of Formula (VI) or a mixture thereof,

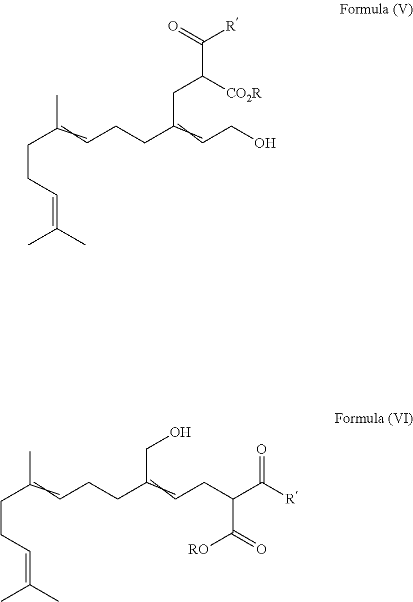

wherein the method comprises contacting a compound of Formula (I), a compound of Formula (II) or a mixture thereof with a compound of Formula (VII) and a palladium catalyst, wherein R is selected from the group consisting of hydrogen and a C1 to C6 alkyl group; and R′ is selected from the group consisting of a C1 to C6 alkyl group and an OR group.

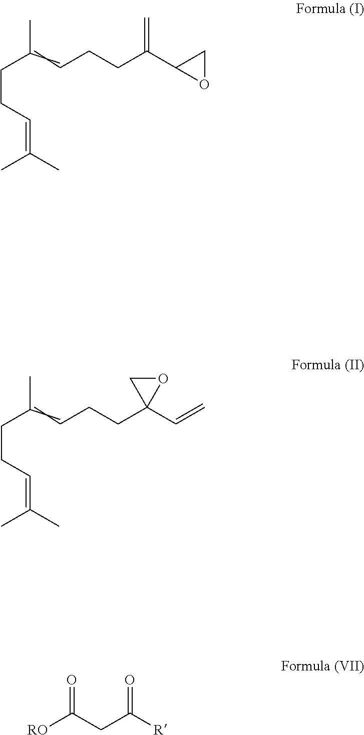

12. The method of paragraph 11, wherein the method produces a mixture of E,E- and E,Z-Formula (V) and/or a mixture of E,E- and E,Z-Formula (VI).

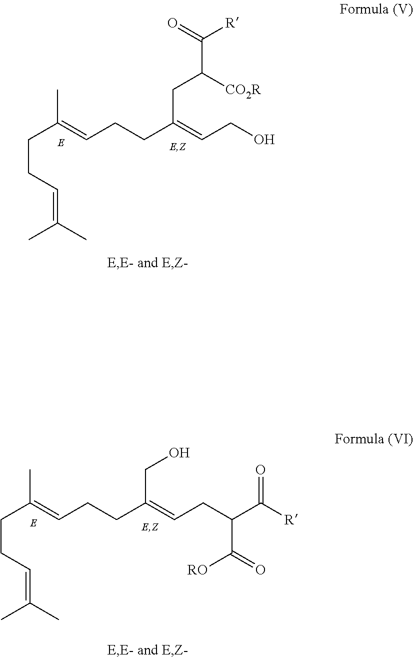

13. The method of any of paragraphs 11 or 12, wherein R′ is methyl.
14. The method of any of paragraphs 11 to 13, wherein the palladium catalyst is selected from a Pd(0) catalyst, such as Pd(PPh₃)₄, Pd₂(dba)₃ or solvated Pd₂(dba)₃ (such as Pd₂(dba)₃∜dba or Pd₂(dba)₃·CHCl₃; a Pd(II) catalyst, such as Pd(OAc)₂, Pd(acac)₂, Pd(MeCN)₂Cl₂, Pd(COD)Cl₂, [(C₃H₅)PdCl]₂, or PdCl₂; or a supported heterogeneous Pd catalyst, such as Pd/C.
15. The method of any of paragraphs 11 to 14, wherein the method produces a mixture of the compounds of Formula (V) and (VI) and further comprises separating the compounds of Formula (V) and (VI).
16. The method of any of paragraphs 11 to 15, wherein the method further comprises separating the E,E- and the E,Z-isomers, for example, by flash chromatography.
17. The method of any of paragraphs 1 to 4, wherein the compound of Formula (V), the compound of Formula (VI) or the mixture thereof is produced by the method of any of paragraphs 11 to 16.
18. A method for making a compound of Formula (X), a compound of Formula (XII), or a mixture thereof.

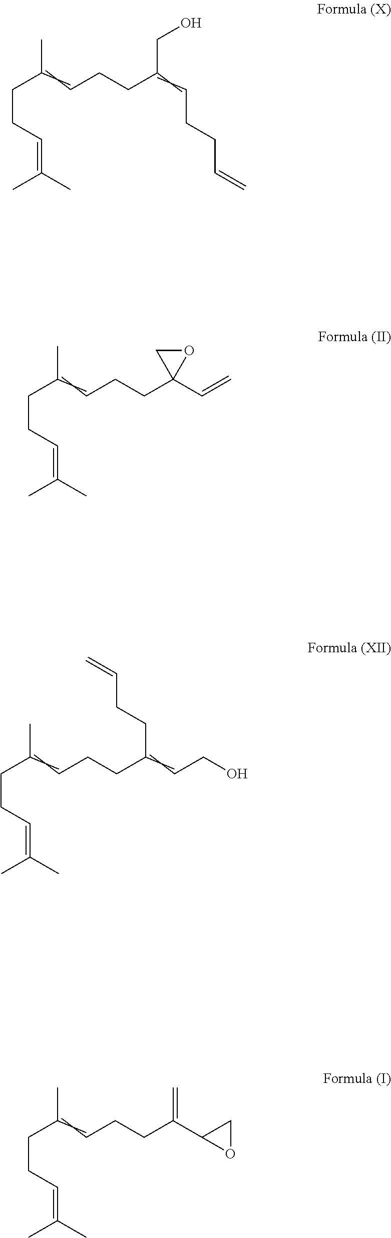 [SMILES: C=CCCC=C(CO)CCC=C(C)CCC=C(C)C ; C=CC1(CCC=C(C)CCC=C(C)C)CO1 ; C=CCCC(=CCO)CCC=C(C)CCC=C(C)C ; C=C(CCC=C(C)CCC=C(C)C)C1CO1]

wherein the method comprises a copper-catalysed alkylation of a compound of Formula (II), a compound of Formula (I), or a mixture thereof.
19. The method of claim 18, wherein the copper-catalysed alkylation of a compound of Formula (II), a compound of Formula (I), or a mixture thereof comprises contacting a compound of Formula (II), a compound of Formula (I), or a mixture thereof with a copper catalyst and a Grignard reagent.
20. The method of paragraph 19, wherein the Grignard reagent is an allyl magnesium halide.
21. The method of paragraph 20, wherein the allyl magnesium halide is allyl magnesium chloride.
22. The method of any of paragraphs 19 to 21, wherein the copper catalyst is CuBr·SMe₂.
23. The method of any of paragraphs 18 to 22, wherein the method produces a mixture of E,E- and E,Z-Formula (X) and/or a mixture of E,E- and E,Z-Formula (XII) and the method further comprises separating the E,E- and the E,Z-isomers

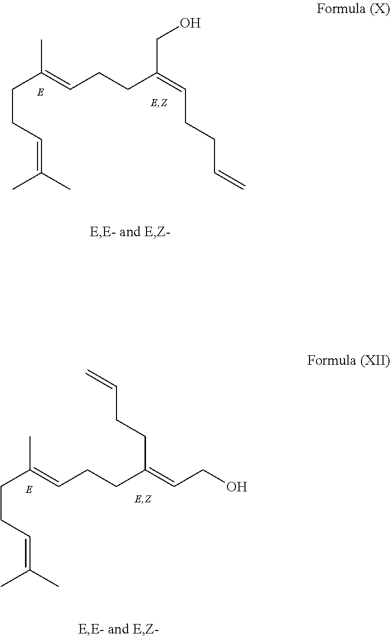 [SMILES: C=CCC/C=C(/CO)CC/C=C(\C)CCC=C(C)C ; C=CCC/C(=C\CO)CC/C=C(\C)CCC=C(C)C]

24. The method of any of paragraphs 7 to 9, wherein the compound of Formula (X), the compound of Formula (XII), or the mixture thereof is produced by the method of any of paragraphs 18 to 23.
25. The method of paragraph 10, wherein the compound of Formula (X), the compound of Formula (XII), or the mixture thereof is produced by the method of any of paragraphs 17 to 21.
26. A method for making a compound of Formula (I), a compound of Formula (II) or a mixture thereof,

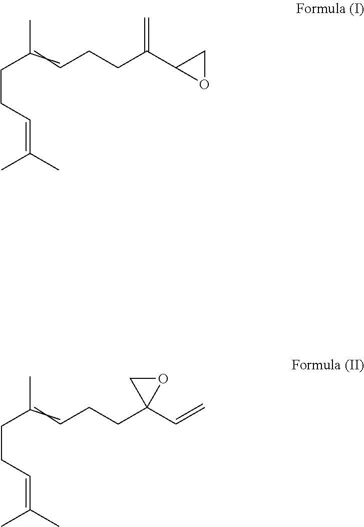 [SMILES: C=C(CCC=C(C)CCC=C(C)C)C1CO1 ; C=CC1(CCC=C(C)CCC=C(C)C)CO1]

wherein the method comprises contacting a compound of Formula (III) with a catalyst comprising a metal complexed with a salen ligand or a porphyrin ligand, wherein the salen ligand has Formula (IV) and wherein the porphyrin ligand has Formula (XI),

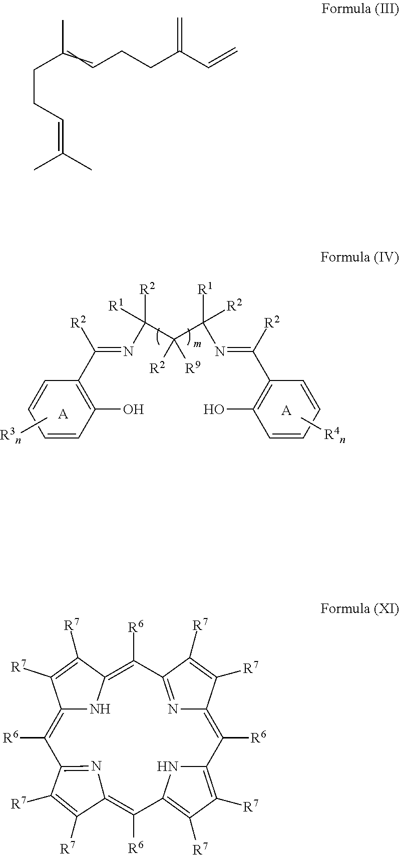

wherein
each ring A is independently selected from phenyl or naphthyl;
each R¹ is independently hydrogen, an alkyl group, an aryl group, or both R¹ together with the carbon atoms to which R¹ are attached form a cycloalkylene group, an arylene group, two or more cycloalkylene groups connected by a single bond or an alkyl group, two or more arylene groups connected by a single bond or an alkyl group, or a cycloalkylene and an arylene group connected by a single bond or an alkyl group;
each R² is independently hydrogen or an alkyl group;
R³ is independently an alkyl group, an alkoxy group, an alkaryl group, an alkylsilyl ether group, or a halide;
R⁴ is independently an alkyl group, an alkoxy group, an alkaryl group, an alkylsilyl ether group, or a halide;
R⁹ is selected from hydrogen, an optionally substituted alkyl group, an optionally substituted aryl group, hydroxyl or an amino group;
each n is selected from 0 to 4;
m is selected from 0 to 4;
each R⁶ is independently selected from hydrogen, an optionally substituted alkyl group, an optionally substituted alkylidene group, an optionally substituted aryl group, or an optionally substituted heteroaromatic group; and
each R⁷ is independently selected from hydrogen, an alkyl group, an ester-containing alkyl group or a carboxylic acid containing alkyl group.
27. The method of paragraph 26, wherein the metal catalyst comprising a salen ligand is catalyst selected from the compounds of Formula (IVa), Formula (IVb), Formula (IVc), and Formula (IVd).

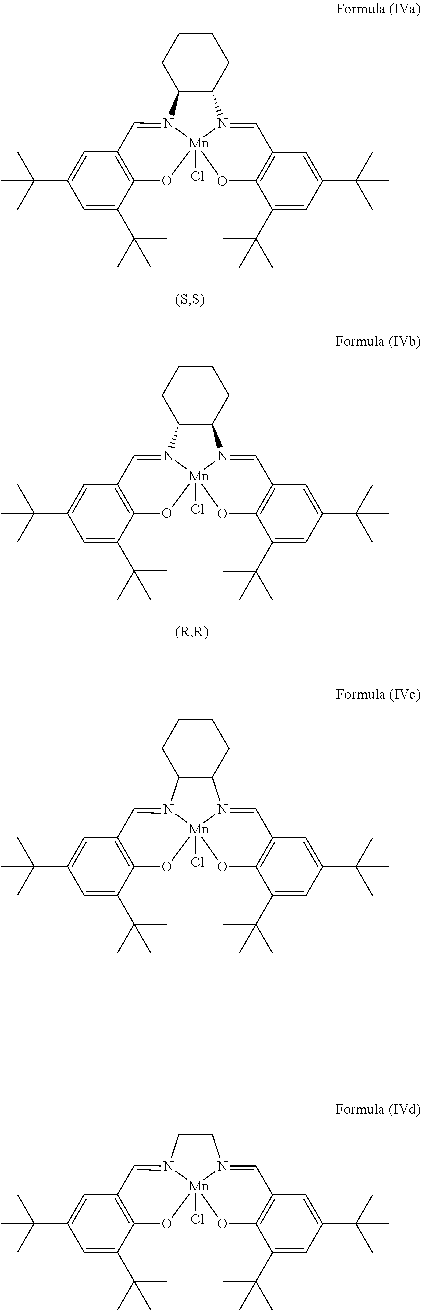

28. The method of any of paragraphs 26 or 27, wherein the compound of Formula (III) is (6E)-ß-farnesene (7,11-dimethyl-3-methylene-1,6E,10-dodecatriene).
29. The method of any of paragraphs 26 to 28, wherein contacting the compound of Formula (III) with a catalyst comprising a metal complexed with a salen ligand or a porphyrin ligand (for example, a salen ligand) comprises contacting the compound of Formula (III) with a catalyst comprising a metal complexed with a salen ligand or a porphyrin ligand (for example, a salen ligand), an oxidant and/or a co-catalyst.
30. The method of any of paragraphs 26 to 29, wherein contacting the compound of Formula (III) with a catalyst comprising a metal complexed with a salen ligand comprises contacting the compound of Formula (III) with a catalyst comprising a metal complexed with a salen ligand and a) aqueous NaOCl, Me₃NO and PhMe; or b) aqueous H₂O₂, NH₄BF₄, Na₃PO₄ and MeOH; or c) aqueous NaOCl and toluene, benzene, chlorobenzene, ethyl acetate, or (trifluoromethyl)benzene.
31. The method of any of paragraphs 26 to 30, wherein the method produces a mixture of the compounds of Formula (I) and (II) and further comprises separating the compounds of Formula (I) and (II), for example, by distillation.
32. A method of separation of a mixture of a compound of Formula (I) and a compound of Formula (II),

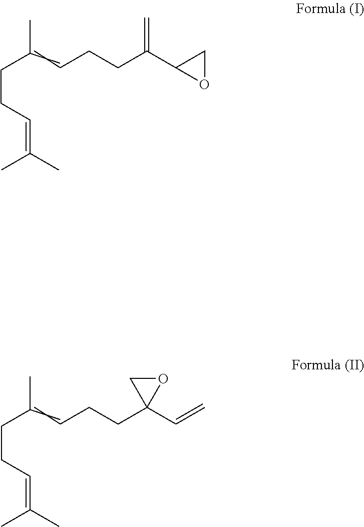 [SMILES: C=C(CCC=C(C)CCC=C(C)C)C1CO1 ; C=CC1(CCC=C(C)CCC=C(C)C)CO1]

wherein the method comprises the distillation of a mixture of a compound of Formula (I) and a compound of Formula (II).
33. The method of paragraph 32, wherein the distillation is vacuum distillation.
34. The method of paragraph 33, wherein the vacuum distillation is at a reduced pressure of from 0.1 mbar to 0.01 mbar.
35. The method of any of paragraphs 32 to 34, wherein the mixture of a compound of Formula (I) and a compound of Formula (II) is produced by a method of any of paragraphs 26 to 30.
36. The method of any of paragraphs 5, 6 or 11 to 25, wherein the compound of Formula (I), the compound of Formula (II) or the mixture thereof is produced by a method of any of paragraphs 26 to 35.
37. A compound of Formula (V), a compound of Formula (VI) or a mixture thereof, wherein R is selected from the group consisting of hydrogen and a C1 to C6 alkyl group; and R′ is selected from the group consisting of a C1 to C6 alkyl group and an OR group.
38. The compound of Formula (V), the compound of Formula (VI) or the mixture thereof of paragraph 36 obtained by or obtainable by the method of any of paragraphs 11 to 17 or 36.
39. A compound of Formula (X), a compound of Formula (XII), or a mixture thereof.
40. The compound of paragraph 39 obtained by or obtainable by the method of any of paragraphs 18 to 23 or 36.
41. A compound of Formula (VIII), a compound of Formula (IX) or a mixture thereof obtained by or obtainable by the method of any of paragraphs 1 to 6, 17 or 36.
42. A compound of Formula (IX) obtained by or obtainable by the method of the any of paragraphs 7 to 10, 24, 25, or 36.
43. A compound of Formula (I), a compound of Formula (II) or a mixture thereof obtained by or obtainable by the method any of paragraphs 26 to 35.
44. A composition comprising a compound of any of paragraphs 37 to 43.
45. Use of a composition of paragraph 44 in a method for making a fragrance composition.
46. A method for making a fragrance composition comprising a method according to any of paragraphs 1 to 36.
47. A method for making a compound of Formula (V), a compound of Formula (VI), or a mixture thereof, according to any of paragraphs 11 to 16, wherein the method comprising contacting a composition comprising a compound of Formula (I), a compound of Formula (II), and a compound of Formula (III).
48. A method for making a compound of Formula (VIII), a compound of Formula (IX), or a mixture thereof according to any of paragraphs 1 to 4, wherein the method comprising contacting a composition further comprising a compound of Formula (III).
49. A method for making a compound of Formula (VIII), a compound of Formula (IX), or a mixture thereof according to any of paragraphs 5 to 10, wherein the method comprising contacting a composition further comprising a compound of Formula (III).
50. The method of paragraph 48, wherein the compound of Formula (V), the compound of Formula (VI) or the mixture thereof is produced by the method of paragraph 47.
51. A method according to paragraph 50 wherein the method is a one-pot procedure.
52. A method according to any of paragraphs 26, 29, 30, 47, 48, and 49 wherein the compound of Formula (III) is replaced by a mixture comprising a compound of Formula (III) and a further isomer thereof, for example a-farnesene (3,7,11-trimethyldodeca-1,3,6,10-tetraene).
53. A method according to paragraph 52 wherein the further isomer is isomerised to a compound of Formula (III).

### Compounds, Compositions and Uses

In another aspect, there is provided a compound of Formula (V), a compound of Formula (VI) or a mixture thereof,

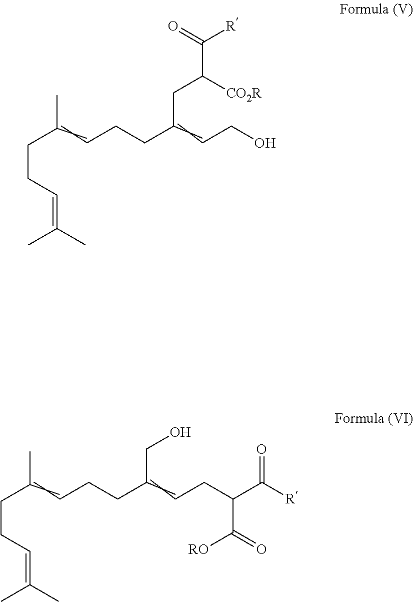

wherein R is selected from the group consisting of hydrogen and C1 to C6 alkyl; and R′ is selected from the group consisting of a C1 to C6 alkyl and an OR group.

The compound of Formula (V), the compound of Formula (VI) or the mixture thereof may be any compound of Formula (V), compound of Formula (VI) or mixture thereof described herein. The compound of Formula (V) may be any compound of Formula (V) described herein. The compound of Formula (VI) may be any compound of Formula (VI) described herein. The mixture of a compound of Formula (V) and a compound of Formula (VI) may be any mixture of a compound of Formula (V) and a compound of Formula (VI) described herein.

In another aspect, there is provided a compound of Formula (V), a compound of Formula (VI) or a mixture thereof obtained by or obtainable by a method described herein, for example, by the method of the fourth aspect. The compound of Formula (V) may be any compound of Formula (V) obtained or obtainable by any method described herein, for example, by the method of the fourth aspect. The compound of Formula (VI) may be any compound of Formula (VI) obtained or obtainable by any method described herein, for example, by the method of the fourth aspect. The mixture of a compound of Formula (V) and a compound of Formula (VI) may be any mixture of a compound of Formula (V) and a compound of Formula (VI) obtained or obtainable by any method described herein, for example, by the method of the fourth aspect.

In another aspect, there is provided a composition comprising a compound of Formula (V), a compound of Formula (VI) or a mixture thereof, wherein R is selected from the group consisting of hydrogen and C1 to C6 alkyl; and R′ is selected from the group consisting of a C1 to C6 alkyl and an OR group. The composition may comprise any compound of Formula (V) described herein, any compound of Formula (VI) described herein or any mixture thereof described herein. In some examples, R′ may be methyl. In some examples, R may be methyl.

The composition may comprise or consist of a compound of Formula (V). The composition may comprise or consist of a compound of Formula (V) as described herein. The composition may comprise or consist of a compound of Formula (V) produced by or producible by a method described herein.

The composition may comprise or consist of a compound of Formula (VI). The composition may comprise or consist of a compound of Formula (VI) as described herein. The composition may comprise or consist of a compound of Formula (VI) produced by or producible by a method described herein.

The composition may comprise or consist of a mixture of a compound of Formula (V) and a compound of Formula (VI). The composition may comprise or consist of a mixture of a compound of Formula (V) and a compound of Formula (VI) as described herein. The composition may comprise or consist of a mixture of a compound of Formula (V) and a compound of Formula (VI) produced by or producible by a method described herein.

In another aspect, there is provided a compound of Formula (X), a compound of Formula (XII), or a mixture thereof.

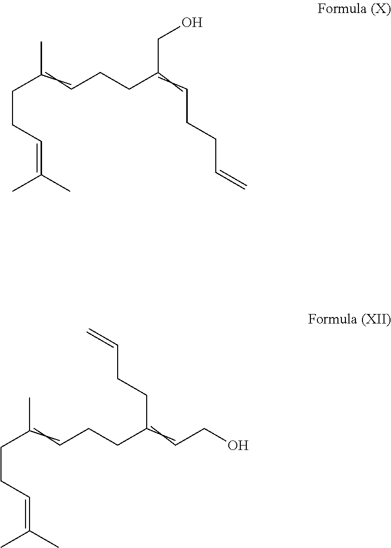 [SMILES: C=CCCC=C(CO)CCC=C(C)CCC=C(C)C ; C=CCCC(=CCO)CCC=C(C)CCC=C(C)C]

The compound of Formula (X) may be any compound of Formula (X) described herein. The compound of Formula (XII) may be any compound of Formula (XII) described herein. The mixture of a compound of Formula (X) and a compound of Formula (XII) may be any mixture of a compound of Formula (X) and a compound of Formula (XII) described herein.

In some examples, the compound of Formula (X) may be any compound of Formula (X) obtained by or obtainable by any method described herein, for example, the method of the fifth aspect. In some examples, the compound of Formula (XII) may be any compound of Formula (XII) obtained by or obtainable by any method described herein, for example, the method of the fifth aspect. In some examples, a mixture of a compound of Formula (X) and a compound of Formula (XII) may be any mixture of a compound of Formula (X) and a compound of Formula (XII) obtained by or obtainable by any method described herein, for example, the method of the fifth aspect.

In another aspect, there is provided a composition comprising a compound of Formula (X), a compound of Formula (XII), or a mixture thereof. The composition may comprise or consist of a compound of Formula (X), a compound of Formula (XII), or a mixture thereof. The composition may comprise or consist of any compound of Formula (X) described herein, any compound of Formula (XII) described herein or any mixture thereof described herein. The composition may comprise or consist of a compound of Formula (X) produced by or producible by a method described herein, a compound of Formula (XII) produced by or producible by a method described herein or a mixture thereof produced by or producible by a method described herein. In another aspect, there is provided a composition comprising a compound of Formula (X). The composition may comprise or consist of a compound of Formula (X). The composition may comprise or consist of any compound of Formula (X) described herein. The composition may comprise or consist of a compound of Formula (X) produced by or producible by a method described herein.

In another aspect, there is provided a compound of Formula (VIII), a compound of Formula (IX), or a mixture thereof obtained by or obtainable by a method described herein, for example, the method of the first aspect or the method of the second aspect. The compound of Formula (VIII) may be obtained by or obtainable by a method described herein, for example, the method of the first aspect or the second aspect. The compound of Formula (IX) may be obtained by or obtainable by a method described herein, for example, the method of the first aspect, the second aspect or the third aspect. The mixture of a compound of Formula (VIII) and a compound of Formula (IX) may be obtained by or obtainable by a method described herein, for example, the method of the first aspect or the second aspect.

The compound of Formula (IX) may be obtained by or obtainable by the method of the third aspect.

In another aspect, there is provided a compound of Formula (II), a compound of Formula (I) or a mixture thereof obtained by or obtainable by a method described herein, for example, by the method of the sixth aspect or the seventh aspect. The compound of Formula (II) may be obtained by or obtainable by the method of the sixth aspect or the seventh aspect. The compound of Formula (I) may be obtained by or obtainable by the method of the sixth aspect or the seventh aspect. The mixture of the compound of Formula (II) and the compound of Formula (I) may be obtained by or obtainable by the method of the sixth aspect. In one aspect, the obtained mixture comprises unreacted compound of Formula (III).

In another aspect, there is also provided the use of a composition described herein in a method for making a fragrance composition. For example, a composition comprising any compound described herein may be used in a method for making a fragrance composition. In some examples, the methods described herein may comprise methods for making a fragrance composition. In some examples, the methods described herein may comprise part of a method for making a fragrance composition. In some examples, there is provided a method for making a fragrance composition comprising a method described herein. In some examples, the method for making a fragrance composition may comprise a method as described in WO patent application number PCT/EP20217059618, wherein, for example, a compound obtained or obtainable by a method described herein is contacted with an SHC enzyme or enzyme variant. In some examples, the method for making a fragrance composition may comprise a method as described in WO patent application number PCT/EP20217059618, wherein the compound of Formula (VIII), the compound of Formula (IX), or a mixture thereof obtained or obtainable by a method described herein is contacted with an SHC enzyme or enzyme variant.

In some examples, a composition comprising or consisting of a compound of Formula (V), a compound of Formula (VI) or a mixture thereof may be used in a method for making a fragrance composition. In some examples, a composition comprising or consisting a compound of Formula (V) may be used in a method for making a fragrance composition. In some examples, a composition comprising or consisting of a compound of Formula (VI) may be used in a method for making a fragrance composition. In some examples, a composition comprising or consisting of a mixture of a compound of Formula (V) and a compound of Formula (VI) may be used in a method for making a fragrance composition.

In some examples, a composition comprising or consisting of a compound of Formula (X), a compound of Formula (XII), or a mixture thereof may be used in a method for making a fragrance composition. In some examples, a composition comprising or consisting of a compound of Formula (X) may be used in a method for making a fragrance composition. In some examples, a composition comprising or consisting of a compound of Formula (XII) may be used in a method for making a fragrance composition. In some examples, a composition comprising or consisting of a mixture of a compound of Formula (X) and a compound of Formula (XII) may be used in a method for making a fragrance composition.

In some examples, a composition comprising or consisting of a compound of Formula (I), a compound of Formula (II) or a mixture thereof may be used in a method for making a fragrance composition. In some examples, a composition comprising or consisting of a compound of Formula (I) may be used in a method for making a fragrance composition.

In some examples, a composition comprising or consisting of a compound of Formula (II) may be used in a method for making a fragrance composition. In some examples, a composition comprising or consisting of a mixture of a compound of Formula (I) and a compound of Formula (II) may be used in a method for making a fragrance composition. In some examples, a composition comprising or consisting of a mixture of a compound of Formula (I) a compound of Formula (II) and a compound of Formula (III) may be used in a method for making a fragrance composition.

In some examples, a composition comprising or consisting of a compound of Formula (VIII), a compound of Formula (IX) or a mixture there of may be used in a method for making a fragrance composition. In some examples, a composition comprising or consisting of a compound of Formula (VIII) may be used in a method for making a fragrance composition. In some examples, a composition comprising or consisting of a compound of Formula (IX) may be used in a method for making a fragrance composition. In some examples, a composition comprising or consisting of a mixture of a compound of Formula (VIII) and a compound of Formula (IX) may be used in a method for making a fragrance composition.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. The term “comprising” also means “including” as well as “consisting” e.g. a composition “comprising” X may consist exclusively of X or may include something additional e.g. X+Y. It must be noted also that, as used in this specification and the appended claims, the singular forms “a”, “an” and “the” include plural referents unless the content clearly dictates otherwise. By way of example, a reference to “an enzyme” is a reference to “one or more enzymes”.

It is to be understood that this disclosure is not limited to the particular methodology, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by the person skilled in the art. In accordance with the present disclosure there may be conventional techniques employed which are within the skill of the art.

This disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting.

Several documents are cited throughout the text of this specification. Each of the documents cited herein (including all patents, patent applications, scientific publications, manufacturer's specifications, instructions, GenBank Accession Number sequence submissions etc.), whether supra or infra, is hereby incorporated by reference in its entirety.

As used herein, “alkyl”, or similar expressions such as “alk” in alkoxy, may refer to a branched, unbranched, or cyclic saturated hydrocarbon, which may, in some examples, contain from about 1 to about 20 carbon atoms, or about 1 to about 10 carbon atoms, or 1 to about 6 carbon atoms.

As used herein, “alkylene” may refer to divalent group derived by removal of two hydrogen atoms from one carbon atom or two hydrogen atoms from different carbon atoms of an alkane, which may, in some examples, contain from about 1 to about 20 carbon atoms, or about 1 to about 10 carbon atoms, or 1 to about 6 carbon atoms.

As used herein, “cycloalkylene” may refer to a cyclic divalent group derived by removal of two hydrogen atoms from one carbon atom or two hydrogen atoms from different carbon atoms of a cycloalkane, which may, in some examples, contain from about 1 to about 20 carbon atoms, or about 1 to about 10 carbon atoms, or 1 to about 6 carbon atoms.

As used herein, “arylene” may refer to a cyclic divalent group derived by removal of two hydrogen atoms from different carbon atoms of an aryl group, which may, in some examples, contain from about 1 to about 20 carbon atoms, or about 1 to about 10 carbon atoms, or 1 to about 6 carbon atoms.

As used herein, “heteroaromatic” may refer to an aromatic hydrocarbon containing at least one heteroatom, which may contain from about 3 to about 20 carbon atoms, or about 5 to 10 carbon atoms and from 1 to about 5 heteroatoms, or 2, or 3 heteroatoms (such as N, O and/or S).

As used herein, the crossed double bond

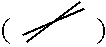

is used to indicates a double bond of unspecified configuration or a mixture comprising compounds of different double bond configurations.

The examples described herein are illustrative of the present disclosure and are not intended to be limitations thereon. Different embodiments of the present disclosure have been described according to the present disclosure. Many modifications and variations may be made to the techniques described and illustrated herein without departing from the spirit and scope of the disclosure. Accordingly, it should be understood that the examples are illustrative only and are not limiting upon the scope of the disclosure.

### EXAMPLES

The following illustrates examples of the methods and other aspects described herein. Thus, these Examples should not be considered as limitations of the present disclosure, but are merely in place to teach how to make examples of the present disclosure.

### Example 1: (E)-2-(4,8-dimethylnona-3,7-dien-1-yl)-2-vinyloxirane (E-Formula (II)) and (E)-2-(6,10-dimethylundeca-1,5,9-trien-2-yl)oxirane (E-Formula (I))

A solution of (E)-beta farnesene (E-β-Formula (III), 27.5 g, 0.135 mol) and Jacobsen's catalyst ((S,S)-(+)-N,N-Bis(3,5-di-tert-butylsalicylidene)-1,2-cyclohexanediamino-manganese(III) chloride, Formula (IVa) [135620-04-1]; 3.42 g, 5.4 mmol, 0.04 eq.) in CH₂Cl₂ (120 ml) was cooled to 0° C. and treated dropwise with a cold aqueous solution (4° C.) of 10% NaOCl (159 g, 0.214 mol, 1.6 eq.). The resulting mixture was allowed to reach room temperature (r.t.), stirred under N₂ at r.t. for 24 h, and extracted with Et₂O (2×100 ml). The combined organic phases were washed with water (2×50 mL) and brine (50 ml), dried over Na₂SO₄, leading after solvent evaporation under reduced pressure to 33.4 g of a crude mixture of (E)-beta farnesene and (E)-beta farnesene epoxides (GC-(E)-2-(4,8-dimethylnona-3,7-dien-1-yl)-2-purity: 8% (E)-beta farnesene, 30% vinyloxirane, 28% (E)-2-(6,10-dimethylundeca-1,5,9-trien-2-yl)oxirane). The crude mixture was purified by chromatography on neutral Al₂O₃ (deactivated, 5% water), elution with 95:5 heptane/AcOEt led to 18.2 g of a 64:36 mixture of (E)-2-(4,8-dimethylnona-3,7-dien-1-yl)-2-vinyloxirane (E-Formula (II))/(E)-2-(6, 10-dimethylundeca-1,5,9-trien-2-yl)oxirane (E-Formula (I)) (GC-purity: 74%).

### Example 2: Isolation of (E)-2-(6,10-dimethylundeca-1,5,9-trien-2-yl)oxirane (E-Formula (I))

A solution of (E)-beta farnesene (E-β-Formula (III) 10.22 g, 0.05 mol) and Jacobsen's catalyst ((S,S)-(+)-N,N-Bis(3,5-di-tert-butylsalicylidene)-1,2-cyclohexanediamino-manganese(III) chloride, Formula (IVa), [135620-04-1]; 1.27 g, 2.0 mmol, 0.04 eq.) in CH₂Cl₂ (50 ml) was cooled to 0° C.and treated dropwise with a cold aqueous solution (4° C.) of 10% NaOCl (45 g, 0.076 mol, 1.5 eq.). The resulting mixture was allowed to reach r.t., stirred under N₂ at r.t. for 24 h, and extracted with Et₂O (2×50 ml). The combined organic phases were washed with water (2×50 mL) and brine (50 ml), dried over Na₂SO₄, leading after solvent evaporation under reduced pressure to 13.40 g of a crude mixture of (E)-beta farnesene and (E)-beta farnesene epoxides. The crude mixture was purified by chromatography on SiO₂; elution with 90:10 heptane/AcOEt (1% Et₃N) led to the loss of (E)-2-(4,8-dimethylnona-3,7-dien-1-yl)-2-vinyloxirane (E-Formula (II)) by decomposition on the column and to the isolation of (E)-2-(6, 10-dimethylundeca-1,5,9-trien-2-yl)oxirane (E-Formula (I)) (2.2 g, 20% yield).

### Example 3: Epoxide Mixture Separated by Distillation

A solution of (E)-beta farnesene (E-β-Formula (III), 120 g, 0.587 mol) and Jacobsen's catalyst ((S,S)-(+)-N,N-Bis(3,5-di-tert-butylsalicylidene)-1,2-cyclohexanediamino-manganese(III) chloride, Formula (IVa), [135620-04-1]; 14.9 g, 23.5 mmol, 0.04 eq.) in CH₂Cl₂ (500 ml) was cooled to 0° C. and treated dropwise with a cold aqueous solution (4° C.) of 10% NaOCl (525 ml, 0.881 mol, 1.5 eq.). The resulting mixture was allowed to reach r.t. (exotherm to 35° C. after 15 min. at RT), stirred under N₂ at r.t. for 24 h, and extracted with pentane (3×50 ml). The combined organic phases were washed with water (2×50 ml) and brine (50 ml), dried over Na₂SO₄, leading after solvent evaporation under reduced pressure to 109.2 g of a crude mixture of (E)-beta farnesene and (E)-beta farnesene epoxides. Wiped-film distillation of the crude mixture led to 75 g of a mixture (E)-beta farnesene and (E)-beta farnesene epoxides that was distilled using a Sulzer column leading to (E)-beta farnesene (8 g,; bp 72° C., 0.06 mbar), (E)-2-(4,8-dimethylnona-3,7-dien-1-yl)-2-vinyloxirane (E-Formula (II), 18.1 g, bp 78° C., 0.03 mbar), (E)-2-(6,10-dimethylundeca-1,5,9-trien-2-yl)oxirane (E-Formula (I), 1 g, bp 83° C., 0.03 mbar), and a mixture of (E)-2-(4,8-dimethylnona-3,7-dien-1-yl)-2-vinyloxirane/(E)-2-(6, 10-dimethylundeca-1,5,9-trien-2-yl)oxirane (8.6 g, approx. 45:55, E-Formula (II)/E-Formula (I)).

### Example 4: Scale-Up of the Distillative Separation

A dark brown liquid (452 g) obtained from (E)-beta farnesene (E-β-Formula (III), 457 g, 2.2 mol) by Jacobsen epoxidation (catalyst Formula (IVc) prepared from cis/trans-1,2-diaminocyclohexane, [694-83-7]), were wiped-film distilled (130° C., 0.05 mbar) to give a residue that was discarded and a light yellow liquid (353 g) that was rectified over a 50×2.5 cm Sulzer EX packed column (3% tocopherol were added as stabilizer). First the unreacted (E)-beta farnesene (E-β-Formula (III)) was collected (72° C., 0.03 mbar) and then the lower boiling (E)-2-(4,8-dimethylnona-3,7-dien-1-yl)-2-vinyloxirane (E-Formula (II)) could be isolated in its pure state. The distillation resulted in equal parts of pure lower boiling epoxide, mixed fractions and pure higher boiling (E)-2-(6, 10-dimethylundeca-1,5,9-trien-2-yl)oxirane (E-Formula (I)). The mixed fractions were redistilled to obtain pure material.

### Example 5: (E)-2-(4,8-dimethylnona-3,7-dien-1-yl)-2-vinyloxirane (E-Formula (II) and (E)-2-(6,10-dimethylundeca-1,5,9-trien-2-yl)oxirane (E-Formula (I))

Using NH₄BF₄/Na₃PO₄ as additives in the presence of H₂O₂ as oxidant (instead of NaOCl) according to: Ebrahimian, G. R.; Mollat du Jourdin, X.; Fuchs, P. L. Org. Lett., 2012, 14, 2630-2633 (Jacobsen Protocols for Large-Scale Epoxidation of Cyclic Dienyl Sulfones: Application to the (+)-Pretazettine Core).

A mixture of (E)-beta farnesene (E-β-Formula (III), 10 g, 48.9 mmol) and MeOH (50 ml) was cooled to 0° C., treated with Na₃PO₄ (3.34 g, 96% pure, 19.57 mmol, 0.4 eq.), NH₄BF₄ (6.35 g, 97% pure, 58.7 mmol, 1.2 eq.), Jacobsen's catalyst ((R,R)-(+)-N,N-Bis(3,5-di-tert-butylsalicylidene)-1,2-cyclohexanediamino-manganese(III) chloride, Formula (IVb), [138124-32-0]; 0.093 g, 0.147 mmol, 0.003 eq.), and the resulting mixture was treated dropwise and slowly with an aqueous solution of 30% H₂O₂ (5 ml, 48.9 mmol, 1.0 eq.), stirred at 5° C. for 1 h, allowed to reach r.t., stirred at r.t. for 24 h, diluted with AcOEt, quenched with aq. Na₂S₂O₄ and extracted three times with AcOEt. The combined organic phases were washed with aq. sat. NaCl, dried over MgSO₄, leading after solvent evaporation under reduced pressure to 10.3 g of a crude mixture of (E)-beta farnesene (E-β-Formula (III)) and (E)-beta farnesene epoxides (1H-NMR: 65:20:14 (E)-beta farnesene/(E)-2-(4,8-dimethylnona-3,7-dien-1-yl)-2-vinyloxirane (E-Formula (II))/(E)-2-(6, 10-dimethylundeca-1,5,9-trien-2-yl)oxirane (E-Formula (I))).

### Example 6a: (E)-2-(4,8-dimethylnona-3,7-dien-1-yl)-2-vinyloxirane (E-Formula (II) and (E)-2-(6, 10-dimethylundeca-1,5,9-trien-2-yl)oxirane (E-Formula (I))

Using Me₃NO·2H₂O as additive in the presence of NaOCl as oxidant and 0.04 eq. Jacobsen's catalyst in AcOEt.

A mixture of (E)-beta farnesene (E-β-Formula (III), 5 g, 24.5 mmol), Jacobsen's catalyst ((R,R)-(+)-N,N-Bis(3,5-di-tert-butylsalicylidene)-1,2-cyclohexanediaminomanganese (III) chloride, Formula (IVb), [138124-32-0], 0.622 g, 0.979 mmol, 0.04 eq.), and Me₃NO·2H₂O (0.549 g, 4.9 mmol, 0.2 eq.) in AcOEt (25 ml) was cooled to 5° C. and treated dropwise with a cold aqueous solution (6° C.) of 15% NaOCl (18.2 g, 36.7 mmol, 1.5 eq.). After 1 h, the resulting mixture was allowed to reach r.t., stirred at r.t. for 19 h, and the aq. phase was extracted with AcOEt (2×25 ml). The combined organic phases were washed with water (2×50 mL) and brine (50 ml), dried over MgSO₄, leading after solvent evaporation under reduced pressure to 5.6 g of a crude mixture of (E)-beta farnesene (E-β-Formula (III)) and (E)-beta farnesene epoxides (GC-purity: 33% (E)-beta farnesene, 23% (E)-2-(4,8-dimethylnona-3,7-dien-1-yl)-2-vinyloxirane (E-Formula (II)), 23% (E)-2-(6,10-dimethylundeca-1,5,9-trien-2-yl)oxirane (E-Formula (I))).

### Example 6b: (E)-2-(4,8-dimethylnona-3,7-dien-1-yl)-2-vinyloxirane (E-Formula (II)) and (E)-2-(6, 10-dimethylundeca-1,5,9-trien-2-yl)oxirane (E-Formula (I))

Using Me₃NO·2H₂O as additive in the presence of NaOCl as oxidant and 0.004 eq. Jacobsen's catalyst in PhMe.

A mixture of (E)-beta farnesene (E-β-Formula (III), 3 g, 14.7 mmol), Jacobsen's catalyst ((R,R)-(+)-N,N-Bis(3,5-di-tert-butylsalicylidene)-1,2-cyclohexanediaminomanganese (III) chloride, Formula (IVb), [138124-32-0]; 0.037 g, 0.059 mmol, 0.004 eq.), and Me₃NO·2H₂O (0.33 g, 2.9 mmol, 0.2 eq.) in PhMe (15 ml) was cooled to 5° C. and treated dropwise with a cold aqueous solution (5° C.) of 15% NaOCl (10.9 g, 22.0 mmol, 1.5 eq.). After 1 h, the resulting mixture was allowed to reach r.t., stirred at r.t. for 28 h, diluted with AcOEt, poured into water and extracted with AcOEt (3×25 ml). The combined organic phases were washed with brine, dried over MgSO₄, leading after solvent evaporation under reduced pressure to 3 g of a crude mixture of (E)-beta farnesene (E-β-Formula (III)) and (E)-beta farnesene epoxides (GC-purity: 46% (E)-beta farnesene, 26% (E)-2-(4,8-dimethylnona-3,7-dien-1-yl)-2-vinyloxirane (E-Formula (II)), 20% (E)-2-(6,10-dimethylundeca-1,5,9-trien-2-yl)oxirane (E-Formula (I)); 1H-NMR: 43:34:23 (E)-beta farnesene/(E)-2-(4,8-dimethylnona-3,7-dien-1-yl)-2-vinyloxirane/(E)-2-(6,10-dimethylundeca-1,5,9-trien-2-yl)oxirane).

### Example 6c: (E)-2-(4,8-dimethylnona-3,7-dien-1-yl)-2-vinyloxirane (E-Formula (II)) and (E)-2-(6, 10-dimethylundeca-1,5,9-trien-2-yl)oxirane (E-Formula (I))

Without Me₃NO·2H₂O and 0.004 eq. Jacobsen's catalyst in PhMe.

A mixture of (E)-beta farnesene (E-β-Formula (III), 3 g, 14.7 mmol), Jacobsen's catalyst ((R,R)-(+)-N,N-Bis(3,5-di-tert-butylsalicylidene)-1,2-cyclohexanediaminomanganese (III) chloride, Formula (IVb), [138124-32-0]; 0.037 g, 0.059 mmol, 0.004 eq.) in PhMe (15 ml) was cooled to 5° C. and treated dropwise with a cold aqueous solution (5° C.) of 15% NaOCl (10.9 g, 22.0 mmol, 1.5 eq.). After 1 h, the resulting mixture was allowed to reach r.t., stirred at r.t. for 47 h, diluted with AcOEt, poured into water and extracted with AcOEt (3×25 ml). The combined organic phases were washed with brine, dried over MgSO₄, leading after solvent evaporation under reduced pressure to 2.8 g of a crude mixture of (E)-beta farnesene (E-β-Formula (III)) and (E)-beta farnesene epoxides (GC-purity: 59% (E)-beta farnesene, 21% (E)-2-(4,8-dimethylnona-3,7-dien-1-yl)-2-vinyloxirane (E-Formula (II)), 17% (E)-2-(6,10-dimethylundeca-1,5,9-trien-2-yl)oxirane (E-Formula (I));

¹H-NMR: 53:28:18 (E)-beta farnesene/(E)-2-(4,8-dimethylnona-3,7-dien-1-yl)-2-vinyloxirane/(E)-2-(6, 10-dimethylundeca-1,5,9-trien-2-yl)oxirane).

### Example 7a: Analytical data of (E)-2-(4,8-dimethylnona-3,7-dien-1-yl)-2-vinyloxirane (E-Formula (II))

¹H NMR (400 MHZ, CDCl₃) δ ppm 5.77 (dd, J=17.4, 10.8 Hz, 1 H), 5.34 (dd, J=17.4, 1.2 Hz, 1 H), 5.20 (dd, J=10.8, 1.2 Hz, 1 H), 5.15-5.05 (m, 2 H), 2.80 (d, J=5.3 Hz, 1 H), 2.65 (d, J=5.4 Hz, 1 H), 2.14-2.02 (m, 4 H), 2.00-1.94 (m, 2 H), 1.78-1.69 (m, 2H), 1.67 (br. s, 3 H), 1.59 (s, 6 H).

¹³C NMR (100 MHZ, CDCl₃) δ ppm 137.43 (d, 1 C), 135.55 (s, 1 C), 131.21 (s, 1 C), 124.18 (d, 1 C), 123.38 (d, 1 C), 116.29 (t, 1 C), 58.40 (s, 1 C), 54.95 (t, 1 C), 39.58 (t, 1 C), 33.54 (t, 1 C), 26.55 (t, 1 C), 25.60 (q, 1 C), 23.50 (t, 1 C), 17.59 (q, 1 C), 15.89 (q, 1 C).

GC-MS (EI): 220 (1, [M]+·), 202 (1), 187 (1), 177 (6), 159 (3), 149 (6), 137 (2), 135 (6), 123 (9), 107 (12), 93 (13), 83 (3), 81 (44), 79 (18), 69 (100), 67 (25), 55 (21), 53 (20), 41 (67), 39 (16).

### Example 7b: Analytical data of (E)-2-(6,10-dimethylundeca-1,5,9-trien-2-yl)oxirane (E-Formula (I)

¹H NMR (400 MHZ, CDCl₃) δ ppm 5.16 (br. s, 1H), 5.15-5.06 (m, 2H), 4.97 (q, J=1.4, 1H), 3.35 (br. dd, J=2.8, 4.0, 1H), 2.88 (dd, J=4.2, 5.6, 1H), 2.65 (dd, J=2.7, 5.6, 1H), 2.21-2.12 (m, 2H), 2.12-1.94 (m, 6H), 1.68 (br. s, 3H), 1.61 (s, 6H).

¹³C NMR (100 MHZ, CDCl₃) δ ppm 145.25 (s, 1 C), 135.59 (s, 1 C), 131.29 (s, 1 C), 124.24 (d, 1 C), 123.51 (d, 1 C), 112.26 (t, 1 C), 53.77 (d, 1 C), 47.91 (t, 1 C), 39.63 (t, 1 C), 30.83 (t, 1 C), 26.63 (t, 1 C), 26.57 (t, 1 C), 25.65 (q, 1 C), 17.65 (q, 1 C), 16.00 (q, 1 C).

GC-MS (EI): 220 (1, [M]+·), 205 (1), 187 (1), 159 (3), 149 (4), 137 (4), 135 (4), 123 (6), 107 (15), 91 (15), 83 (3), 81 (25), 79 (17), 69 (100), 67 (18), 55 (18), 53 (16), 41 (62), 39 (16).

### Example 8: (E)-2-(4,8-dimethylnona-3,7-dien-1-yl)-2-vinyloxirane (E-Formula (II) and (E)-2-(6,10-dimethylundeca-1,5,9-trien-2-yl)oxirane (E-Formula (I)) prepared in ethyl acetate at reflux conditions

A solution of (E)-beta farnesene (E-β-Formula (III), 50 g, 0.245 mol) and Jacobsen's catalyst N,N-Bis(3,5-di-tert-butylsalicylidene)-1,2-ethanediaminomanganese(III) chloride, Formula (IVd); 2.84 g, 4.89 mmol, 0.02 eq.) and tetrabutylammonium bromide (0.5 g, 1.55 mmol) in EtOAc (450 ml) was heated to reflux (77° C. and treated dropwise with an aqueous solution of 10% NaOCl (182 g, 0.367 mol, 1.5 eq.) over the course of 3.5 hours. Stirring was continued at reflux for 1 hour after the addition was complete.

The resulting mixture was allowed to reach room temperature (r.t.) and then the layers were separated. The dark brown, organic layer was washed twice with saturated NH₄Cl solution (200 ml) and then transferred to an Erlenmeyer flask (1000 ml). Water (160 ml) and H₂O₂ (35%) and tetrabutylammonium bromide (0.5 g, 1.55 mmol) were added. The reaction was exothermal, the temperature rose to 35° C.and gas evolution was observed.

The mixture was stirred at rt. for 1.5 hours resulting in a clear brown mixture. The layers were separated and the organic solution was washed once with sodium metabisulfite solution (5%, 250 ml) and twice with water/brine (2:1, 300 ml) and then one more time with brine (100 ml). The solution was dried over MgSO₄ and concentrated in vacuum to give a clear, brown liquid (57.8 g). The crude product mixture was wipe-film distilled (85° C., 0.04 mbar) to give 42.3 g of a colourless mixture containing (E)-beta farnesene and (E)-beta farnesene epoxides (GC-purity: 29% (E)-beta farnesene, 32% (E)-2-(4,8-dimethylnona-3,7-dien-1-yl)-2-vinyloxirane (E-Formula (II)), 22% (E)-2-(6,10-dimethylundeca-1,5,9-trien-2-yl)oxirane) (E-Formula (I)). The crude mixture was used as is for the subsequent steps.

### Example 9: Methyl (4Z,8E)-2-acetyl-5-(hydroxymethyl)-9,13-dimethyltetradeca-4,8,12-trienoate (E,Z-Formula (VI) and Methyl (4E,8E)-2-acetyl-5-(hydroxymethyl)-9,13-dimethyltetradeca-4,8,12-trienoate (E,E-Formula (VI))

A solution of Pd(PPh₃)₄ (21.0 mg, 0.018 mmol, 0.02 eq.) in THF (20 ml) was treated at r.t. with (E)-2-(4,8-dimethylnona-3,7-dien-1-yl)-2-vinyloxirane (E-Formula (II), 200 mg, 0.91 mmol), stirred for 5 min and the resulting solution treated with methyl acetoacetate (Formula (VII), R=Me, R′=Me; 158 mg, 1.36 mmol, 1.5 eq.) and heated at 50° C. for 2 h. The resulting mixture was cooled to r.t. and treated with aq. saturated NaCl solution. The organic phase was separated, washed with aq. saturated NaCl solution (2×20 ml) and dried over Na₂SO₄. After solvent evaporation under reduced pressure a crude mixture (300 mg) was isolated which was purified by flash chromatography (petrol ether/Et₂O 1:1) on SiO₂ yielding methyl (4Z,8E)-2-acetyl-5-(hydroxymethyl)-9,13-dimethyltetradeca-4,8, 12-trienoate (E,Z-Formula (VI), R=Me, R′=Me, 170 mg, 55% yield) and methyl (4E,8E)-2-acetyl-5-(hydroxymethyl)-9,13-dimethyltetradeca-4,8,12-trienoate (E,E-Formula (VI), R=Me, R′=Me, 30 mg, 9% yield).

Methyl (4Z,8E)-2-acetyl-5-(hydroxymethyl)-9, 13-dimethyltetradeca-4,8, 12-trienoate ¹H NMR (500 MHZ, CDCl₃) δ ppm 5.13 (t, J=7.7, 1H), 5.08-5.03 (m, 2H), 4.13 (d, J=11.9, 1H), 4.07 (d, J=12.2, 1H), 3.70 (s, 3H), 3.52 (t, J=7.3, 1H), 2.66-2-54 (m, 2H), 2.40-2.20 (br. s, OH), 2.21 (s, 3H, MeCO), 2.14-1.97 (m, 6H), 1.96-1.90 (m, 2H), 1.65 (br. s, 3H), 1.57 (s, 3H), 1.56 (s, 3H).

¹³C NMR (125 MHZ, CDCl₃) δ ppm 202.90 (s, 1 C, C═O), 170.09 (s, 1 C, CO₂), 142.06 (s, 1 C), 135.47 (s, 1 C), 131.33 (s, 1 C), 124.29 (d, 1 C), 123.74 (d, 1 C), 123.25 (d, 1 C), 60.02 (t, 1 C, CH₂OH), 59.39 (d, 1 C), 52.56 (q, 1 C, OMe), 39.71 (t, 1 C), 35.54 (t, 1 C), 29.47 (q, 1 C, MeCO), 26.73 (t, 2 C), 26.48 (t, 1 C), 25.70 (q, 1 C), 17.68 (q, 1 C), 16.07 (q, 1 C).

GC-MS (EI): 336 (1), 175 (4), 167 (5), 159 (8), 139 (19), 133 (17), 121 (12), 107 (15), 93 (14), 81 (31), 79 (21), 69 (100), 67 (17), 59 (5), 55 (16), 43 (62), 41 (56), 31 (2).

Methyl (4E,8E)-2-acetyl-5-(hydroxymethyl)-9,13-dimethyltetradeca-4,8, 12-trienoate:

¹H NMR (400 MHZ, CDCl₃) δ ppm 5.31 (t, J=7.3, 1H), 5.15-5.03 (m, 2H), 4.01 (s, 2H), 3.73 (s, 3H), 3.48 (t, J=7.5, 1H), 2.65-2-57 (m, 2H), 2.23 (s, 3H, MeCO), 2.16-2.01 (m, 6H), 2.01-1.94 (m, 2H), 1.92-1.85 (br. s, OH), 1.67 (br. s, 3H), 1.59 (s, 6H).

¹³C NMR (100 MHZ, CDCl₃) δ ppm 202.57 (s, 1 C, C═O), 169.81 (s, 1 C, CO₂), 141.98 (s, 1 C), 135.83 (s, 1 C), 131.35 (s, 1 C), 124.17 (d, 1 C), 123.48 (d, 1 C), 121.23 (d, 1 C), 66.50 (t, 1 C, CH₂OH), 59.39 (d, 1 C), 52.41 (q, 1 C, OMe), 39.63 (t, 1 C), 29.13 (q, 1 C, MeCO), 28.08 (t, 1 C), 26.67 (t, 1 C), 26.60 (t, 1 C), 26.14 (t, 1 C), 25.62 (q, 1 C), 17.62 (q, 1 C), 15.96 (q, 1 C).

GC-MS (EI): 336 (1), 318 (1), 303 (1), 175 (4), 167 (4), 159 (6), 139 (12), 133 (16), 121 (10), 107 (12), 95 (13), 93 (13), 81 (32), 79 (19), 69 (100), 67 (17), 59 (5), 55 (14), 43 (56), 41 (53), 31 (2).

### Example 10: Methyl (4Z,8E)-2-acetyl-5-(hydroxymethyl)-9,13-dimethyltetradeca-4,8,12-trienoate (E,Z-Formula (VI), R=Me, R′=Me), Methyl (4E,8E)-2-acetyl-5-(hydroxymethyl)-9,13-dimethyltetradeca-4,8,12-trienoate (E,E-Formula (VI), R=Me, R′=Me), Methyl (4Z,7E)-2-acetyl-4-(2-hydroxyethylidene)-8,12-dimethyltrideca-7,11-dienoate (E,Z-Formula (V), R=Me, R′=Me) and Methyl (4E,7E)-2-acetyl-4-(2-hydroxyethylidene)-8,12-dimethyltrideca-7.11-dienoate (E,E-Formula (V), R=Me, R′=Me)

A solution of Pd(PPh₃)₄ (1.05 g, 0.908 mmol, 0.1 eq.) in THF (250 ml) was treated at r.t. with a 1:1 mixture of (E)-2-(4,8-dimethylnona-3,7-dien-1-yl)-2-vinyloxirane (E-Formula (II))/(E)-2-(6,10-dimethylundeca-1,5,9-trien-2-yl)oxirane (E-Formula (I)) (2 g, 9.08 mmol), stirred for 5 min and the resulting solution treated with methyl acetoacetate (Formula (VII), R=Me, R′=Me; 3.16 g, 27.2 mmol, 3 eq.) and stirred at r.t. for 24 h. The resulting mixture was treated with aq. saturated NaCl solution, the organic phase was separated and dried over Na₂SO₄. After solvent evaporation under reduced pressure a crude mixture was isolated which was purified by flash chromatography (heptane/AcOEt 9:1 to 1:9) on SiO₂ yielding methyl (4Z,8E)-2-acetyl-5-(hydroxymethyl)-9,13-dimethyltetradeca-4,8, 12-trienoate (E,Z-Formula (VI), R=Me, R′=Me; 0.79 g, 25% yield) and methyl (4Z,7E)-2-acetyl-4-(2-hydroxyethylidene)-8, 12-dimethyltrideca-7,11-dienoate (E,Z-Formula (V), R=Me, R′=Me; 410 mg, 13% yield) and a mixture of methyl (4E,8E)-2-acetyl-5-(hydroxymethyl)-9,13-dimethyltetradeca-4,8,12-trienoate (E,E-Formula (VI), R=Me, R′=Me) and methyl (4E,7E)-2-acetyl-4-(2-hydroxyethylidene)-8,12-dimethyltrideca-7,11-dienoate (E,E-Formula (V), R=Me, R′=Me).

Analytical data for methyl (4Z,8E)-2-acetyl-5-(hydroxymethyl)-9,13-dimethyltetradeca-4,8, 12-trienoate and methyl (4E,8E)-2-acetyl-5-(hydroxymethyl)-9,13-dimethyl-tetradeca-4,8,12-trienoate are described in Example 9.

Methyl (4Z,7E)-2-acetyl-4-(2-hydroxyethylidene)-8,12-dimethyltrideca-7,11-dienoate:

¹H NMR (500 MHZ, CDCl₃) δ ppm 5.51 (t, J=7.2, 1H), 5.07-5.01 (m, 2H), 4.15-4.02 (m, 2H), 3.69 (s, 3H), 3.66 (dd, J=6.7, 8.5, 1H), 2.66 (dd, J=8.8, 14.0, 1H), 2.54 (dd, J=6.7, 14.0, 1H), 2.40-2.23 (br. s, OH), 2.20 (s, 3H, MeCO), 2.12-1.88 (m, 8H), 1.63 (br. s, 3H), 1.55 (s, 6H).

¹³C NMR (125 MHZ, CDCl₃) δ ppm 202.43 (s, 1 C, C═O), 169.99 (s, 1 C, CO₂), 138.20 (s, 1 C), 135.66 (s, 1 C), 131.18 (s, 1 C), 127.02 (d, 1 C), 124.06 (d, 1 C), 123.08 (d, 1 C), 58.33 (t, 1 C, CH₂OH), 57.48 (d, 1 C), 52.37 (q, 1 C, OMe), 39.49 (t, 1 C), 35.65 (t, 1 C), 29.26 (q, 1 C, MeCO), 28.29 (t, 1 C), 26.50 (t, 1 C), 26.21 (t, 1 C), 25.51 (q, 1 C), 17.50 (q, 1 C), 15.88 (q, 1 C).

Methyl (4E,7E)-2-acetyl-4-(2-hydroxyethylidene)-8, 12-dimethyltrideca-7,11-dienoate:

¹H NMR (400 MHZ, CDCl₃) δ ppm 5.34 (t, J=6.9, 1H), 5.12-4.99 (m, 2H), 4.05 (d, J=6.9, 2H), 3.68 (s, 3H), 3.64 (t, J=7.6, 1H), 2.60-2.52 (m, 2H), 2.19 (s, 3H, MeCO), 2.25-2.04 (br. s, OH), 2.12-1.87 (m, 8H), 1.63 (br. s, 3H), 1.55 (s, 6H).

¹³C NMR (100 MHZ, CDCl₃) δ ppm 202.39 (s, 1 C, C═O), 169.73 (s, 1 C, CO2), 138.70 (s, 1 C), 136.05 (s, 1 C), 131.23 (s, 1 C), 126.33 (d, 1 C), 124.00 (d, 1 C), 122.95 (d, 1 C), 58.58 (t, 1 C, CH₂OH), 57.94 (d, 1 C), 52.30 (q, 1 C, OMe), 39.49 (t, 1 C), 34.44 (t, 1 C), 30.25 (t, 1 C), 28.85 (q, 1 C, MeCO), 26.63 (t, 1 C), 26.46 (t, 1 C), 25.50 (q, 1 C), 17.50 (q, 1 C), 15.87 (q, 1 C).

### Example 11: (5Z,9E)-6-(hydroxymethyl)-10,14-dimethylpentadeca-5,9,13-trien-2-one

A mixture of methyl (4Z,8E)-2-acetyl-5-(hydroxymethyl)-9,13-dimethyltetradeca-4,8,12-trienoate (E,Z-Formula (VI), R=Me, R′=Me, 1.7 g, 5.05 mmol) and 10% aqueous NaOH solution (40 ml) was heated at reflux for 1 h. The resulting mixture was cooled to r.t., neutralized to pH 7 by addition of aq. saturated NH₄Cl solution, and extracted with Et₂O. The organic phase was separated and dried over Na₂SO₄, leading after solvent evaporation under reduced pressure to 2.0 g of a crude mixture as a dark oil. Purification by Kugelrohr distillation (175° C., 0.018 mbar) yielded (5Z,9E)-6-(hydroxymethyl)-10,14-dimethylpentadeca-5,9,13-trien-2-one (E,Z-Formula (IX), R′=Me, 0.855 g, 60% yield) as an oil.

¹H NMR (400 MHZ, CDCl₃) δ ppm 5.17 (t, J=7.6, 1H), 5.13-5.05 (m, 2H), 4.14 (s, 2H), 3.00-2.55 (br. s, OH), 2.55 (t, J=6.7, 2H), 2.35 (dt, J=6.9, 7.1, 2H), 2.13 (s, 3H, MeCO), 2.17-2.01 (m, 6H), 2.00-1.92 (m, 2H), 1.67 (br. s, 3H), 1.60 (br. s, 3H), 1.59 (br. s, 3H).

¹³C NMR (100 MHZ, CDCl₃) δ ppm 209.02 (s, 1 C, C═O), 139.79 (s, 1 C), 135.30 (s, 1 C), 131.27 (s, 1 C), 126.65 (d, 1 C), 124.26 (d, 1 C), 123.85 (d, 1 C), 60.20 (t, 1 C), 43.19 (t, 1 C), 39.66 (t, 1 C), 35.60 (t, 1 C), 30.15 (q, 1 C), 26.74 (t, 1 C), 26.69 (t, 1 C), 25.66 (q, 1 C), 21.73 (t, 1 C), 17.65 (q, 1 C), 16.01 (q, 1 C).

GC-MS (EI): 278 (1), 260 (1, [M-H₂O]^{+•}), 245 (1), 217 (3), 202 (1), 178 (5), 159 (4), 141 (5), 137 (4), 133 (19), 123 (13), 105 (11), 95 (16), 93 (16), 81 (34), 79 (15), 69 (100), 67 (19), 55 (15), 43 (87), 41 (62), 31 (1).

### Example 12: (5E,9E)-6-(hydroxymethyl)-10,14-dimethylpentadeca-5,9,13-trien-2-one:

A mixture of methyl (4E,8E)-2-acetyl-5-(hydroxymethyl)-9,13-dimethyltetradeca-4,8, 12-trienoate (E,E-Formula (VI), R=Me, R′=Me, 0.811 g, 2.4 mmol) and 10% aqueous NaOH solution (10 ml) was heated at reflux for 1 h. The resulting mixture was cooled to r.t., extracted with Et₂O and the organic phase was dried over Na₂SO₄, leading after solvent evaporation under reduced pressure to 0.602 g of a crude mixture as a dark oil. Purification by flash chromatography on SiO₂ yielded (5E,9E)-6-(hydroxymethyl)-10, 14-dimethylpentadeca-5,9,13-trien-2-one (E,E-Formula (IX), R′=Me, 0.455 g, 67% yield) as an oil.

¹H NMR (400 MHZ, CDCl₃) δ ppm 5.33 (t, J=7.2, 1H), 5.13-5.01 (m, 2H), 3.98 (br. s, 2H), 2.46 (t, J=7.3, 2H), 2.29 (dt, J=7.1, 7.3, 2H), 2.22-2.05 (br. s, OH), 2.10 (s, 3H, MeCO), 2.13-1.98 (m, 6H), 1.97-1.90 (m, 2H), 1.64 (br. s, 3H), 1.56 (br. s, 6H).

¹³C NMR (100 MHZ, CDCl₃) δ ppm 208.37 (s, 1 C), 139.91 (s, 1 C), 135.46 (s, 1 C), 131.19 (s, 1 C), 124.42 (d, 1 C), 124.11 (d, 1 C), 123.60 (d, 1 C), 66.54 (t, 1 C), 43.40 (t, 1 C), 39.57 (t, 1 C), 29.75 (q, 1 C), 27.96 (t, 1 C), 26.75 (t, 1 C), 26.51 (t, 1 C), 25.52 (q, 1 C), 21.68 (t, 1 C), 17.51 (q, 1 C), 15.84 (q, 1 C). GC-MS (EI): 278 (1), 260 (1, [M-H₂O]^{+•}), 245 (1), 217 (3), 202 (2), 178 (5), 159 (5), 141 (6), 137 (5), 133 (24), 123 (12), 105 (11), 95 (16), 93 (17), 81 (36), 69 (100), 67 (18), 58 (3), 55 (15), 53 (13), 43 (85), 41 (63), 31 (1).

### Example 13: (5Z,8E)-5-(2-hydroxyethylidene)-9,13-dimethyltetradeca-8,12-dien-2-one and (5E,8E)-5-(2-hydroxyethylidene)-9,13-dimethyltetradeca-8,12-dien-2-one

Step a) A mixture of a 20:70 mixture of (E)-2-(4,8-dimethylnona-3,7-dien-1-yl)-2-vinyloxirane (E-Formula (II))/(E)-2-(6, 10-dimethylundeca-1,5,9-trien-2-yl)oxirane (E-Formula (I)) (1 g, 4.54 mmol), methyl acetoacetate (Formula (VII), R=Me, R′=Me; 1.17 g, 9.98 mmol, 2.2 eq.), palladium (II) bis(acetylacetonate) (1.9 mg, 0.0091 mmol, 0.002 eq.), triphenylphosphine (9.6 mg, 0.036 mmol, 0.008 eq.), and potassium carbonate (1.28 g, 9.08 mmol, 2.0 eq.) in THF (9 ml) was stirred at 40° C. for 24 h. The reaction mixture was cooled to r.t., treated with water and extracted three time with EtOAc. The organic phases were combined, washed with aq. saturated NaCl solution, dried over Na₂SO₄, leading after solvent evaporation under reduced pressure to a crude mixture (1.9 g as a yellow oil).

Step b) A solution of the crude product obtained in the previous step (1.89 g) in MeOH (5.3 ml) was treated with 2M aq. KOH (5.6 ml, 11.3 mmol) and heated at 40° C. for 6 h. The reaction mixture was cooled to r.t., diluted with EtOAc, quenched with 2M aq. HCl and the aq. phase was extracted with EtOAc. The organic phases were combined, dried over MgSO₄, leading after solvent evaporation under reduced pressure to a crude mixture (1.24 g as a brown oil; ¹H-NMR: 20% (5Z,9E)-6-(hydroxymethyl)-10,14-dimethylpentadeca-5,9, 13-trien-2-one (E,Z-Formula (IX), R′=Me) and 80% of a 80:20 mixture of (5Z,8E)-5-(2-hydroxyethylidene)-9,13-dimethyltetradeca-8,12-dien-2-one (E,Z-Formula (VIII), R′=Me) and (5E,8E)-5-(2-hydroxyethylidene)-9,13-dimethyltetradeca-8, 12-dien-2-one (E,E-Formula (VIII), R′=Me). Purification by flash chromatography (heptane/AcOEt 1:0 to 2:8) on SiO2 yielded first a mixture of (5Z,9E)-6-(hydroxymethyl)-10, 14-dimethylpentadeca-5,9,13-trien-2-one (56%) and (5Z,8E)/(5E,8E)-isomers (44%, 2:1) (0.19 g, 15% yield) and a second fraction of a mixture of (5Z,8E)-5-(2-hydroxyethylidene)-9, 13-dimethyltetradeca-8, 12-dien-2-one (E,Z-Formula (VIII), R′=Me) and (5E,8E)-5-(2-hydroxyethylidene)-9,13-dimethyltetra-deca-8, 12-dien-2-one (E,E-Formula (VIII), R′=Me, 0.48 g, 38% yield) in a 2:1 ratio.

(5Z,8E)-5-(2-hydroxyethylidene)-9,13-dimethyltetradeca-8, 12-dien-2-one:

¹H NMR (400 MHZ, CDCl₃) δ ppm 5.45 (t, J=7.2, 1H), 5.10-5.02 (m, 2H), 4.11 (d, J=7.1, 2H), 2.53 (t, J=7.4, 2H), 2.32 (t, J=7.4, 2H), 2.11 (s, 3H, MeCO), 2.10-1.90 (m, 8H), 1.65 (br. s, 3H), 1.57 (s, 6H).

¹³C NMR (100 MHZ, CDCl₃) δ ppm 208.47 (s, 1 C), 141.50 (s, 1 C), 135.45 (s, 1 C), 131.19 (s, 1 C), 124.81 (d, 1 C), 124.15 (d, 1 C), 123.42 (d, 1 C), 58.50 (t, 1 C), 41.89 (t, 1 C), 39.56 (t, 1 C), 36.14 (t, 1 C), 29.96 (q, 1 C), 26.57 (t, 1 C), 26.34 (t, 1 C), 25.56 (q, 1 C), 23.96 (t, 1 C), 17.55 (q, 1 C) 15.92 (q, 1 C).

(5E,8E)-5-(2-hydroxyethylidene)-9,13-dimethyltetradeca-8,12-dien-2-one:

¹H NMR (400 MHZ, CDCl₃) δ ppm 5.35 (br. t, J=6.9, 1H), 5.10-5.02 (m, 2H), 4.10 (d, J=6.9, 2H), 2.57-2.52 (m, 2H), 2.28 (t, J=7.1, 2H), 2.13 (s, 3H, MeCO), 2.10-1.90 (m, 8H), 1.65 (br. s, 3H), 1.57 (s, 6H).

¹³C NMR (100 MHZ, CDCl₃) δ ppm 208.23 (s, 1 C), 141.48 (s, 1 C), 135.92 (s, 1 C), 131.28 (s, 1 C), 124.27 (d, 1 C), 124.08 (d, 1 C), 123.25 (d, 1 C), 58.77 (t, 1 C), 41.93 (t, 1 C), 39.56 (t, 1 C), 30.64 (t, 1 C), 30.16 (t, 1 C), 29.80 (q, 1 C), 26.81 (t, 1C), 26.52 (t, 1 C), 25.56 (q, 1 C), 17.55 (q, 1 C), 15.89 (q, 1 C).

Mixture of (5Z,8E)- and (5E,8E)-5-(2-hydroxyethylidene)-9,13-dimethyltetradeca-8, 12-dien-2-one:

GC-MS (EI) : 260 (1, [M]^{+•}-H₂O), 217 (2), 209 (1), 202 (2), 191 (4), 175 (4), 159 (4), 149 (5), 133 (32), 121 (12), 105 (16), 93 (21), 91 (19), 81 (32), 79 (16), 69 (100), 67 (16), 55 (15), 43 (65), 41 (59).

### Example 14: (5Z,9E)-6-(hydroxymethyl)-10,14-dimethylpentadeca-5,9,13-trien-2-one

(E,Z-Formula (IX), R′=Me), (5E,9E)-6-(hydroxymethyl)-10, 14-dimethylpentadeca-5,9,13-trien-2-one (E,E-Formula (IX), R′=Me), (5Z,8E)-5-(2-hydroxyethylidene)-9,13-dimethyltetradeca-8.12-dien-2-one (E,Z-Formula (VIII), R′=Me) and (5E,8E)-5-(2-hydroxyethylidene)-9,13-dimethyltetradeca-8,12-dien-2-one (E,E-Formula (VIII), R′=Me)

Step a): A mixture of a 64:36 mixture of (E)-2-(4,8-dimethylnona-3,7-dien-1-yl)-2-vinyloxirane (E-Formula (II))/(E)-2-(6,10-dimethylundeca-1,5,9-trien-2-yl)oxirane (E-Formula (I)), (2 g, 9.08 mmol), methyl acetoacetate (Formula (VII), R=Me, R′=Me, 2.13 g, 18.15 mmol, 2.0 eq.), bis(acetonitrile)palladium (II) dichloride (1.190 mg, 0.00454 mmol, 0.0005 eq.), 1,1′-bis(diphenylphosphino)ferrocene [CAS 12150-46-8] (5.18 mg, 0.009 mmol, 0.001 eq.), and potassium carbonate (2.56 g, 18.15 mmol, 2.0 eq.) in THF (8 ml) was stirred at 40° C. for 24 h. The reaction mixture was cooled to r.t., treated with water and extracted three time with EtOAc. The organic phases were combined, washed with aq. saturated NaCl solution, dried over Na₂SO₄, leading after solvent evaporation under reduced pressure to a crude mixture (3.45 g as an orange oil).

Step b): A mixture of the crude product obtained in the previous step (3.34 g), 32% aq. NaOH (4.37 ml, 34.9 mmol), and water (4.37 ml) was heated at reflux for 1 h. The reaction mixture was cooled to r.t. and extracted three times with MTBE (methyl tert-butyl ether). The combined organic phases were washed with aq. saturated NH₄Cl solution and with aq. saturated NaCl solution, dried over MgSO₄, leading after solvent evaporation under reduced pressure to a crude mixture (2.16 g as an orange oil; ¹H-NMR: 32:27 (5E,9E)-6-(hydroxymethyl)-10, 14-dimethylpentadeca-5,9,13-trien-2-one/(5Z,9E)-6-(hydroxymethyl)-10, 14-dimethylpentadeca-5,9,13-trien-2-one and 16:24 (5Z,8E)-5-(2-hydroxyethylidene)-9,13-dimethyltetradeca-8,12-dien-2-one/(5E,8E)-5-(2-hydroxyethylidene)-9, 13-dimethyltetradeca-8, 12-dien-2- one. Purification by bulb-to-bulb (Kugelrohr) distillation (180° C., 0.04-0.01mbar) yielded a distilled fraction of isomers (1.53 g, 91% purity, 57% yield over two steps).

### Example 15: (5Z,9E)-6-(hydroxymethyl)-10,14-dimethylpentadeca-5,9,13-trien-2-one (E,Z-Formula (IX), R′=Me), (5E,9E)-6-(hydroxymethyl)-10,14-dimethylpentadeca-5,9,13-trien-2-one (E,E-Formula (IX), R′=Me), (5Z,8E)-5-(2-hydroxyethylidene)-9,13-dimethyltetradeca-8,12-dien-2-one (E,Z-Formula (VIII), R′=Me) and (5E,8E)-5-(2-hydroxyethylidene)-9,13-dimethyltetradeca-8,12-dien-2-one (E,E-Formula (VIII), R′=Me)

Step a): In a 25 ml two-neck flask equipped with a magnetic stirrer, a reflux condenser and a thermometer, a mixture of beta farnesene/(E)-2-(4,8-dimethylnona-3,7-dien-1-yl)-2-vinyloxirane (E-Formula (II))/(E)-2-(6, 10-dimethylundeca-1,5,9-trien-2-yl)oxirane (E-Formula (I)) (1 g; 32% beta farnesene, 28% E-Formula (II), 23% E-Formula (I); 2.36 mmol epoxides), methyl acetoacetate ((Formula (VII), R=Me, R′=Me; 0.554 g, 4.7 mmol, 2.0 eq.), palladium (II) bis(acetylacetonate) (2 mg, 0.009 mmol, 0.004 eq.), PPh₃ (10 mg, 0.038 mmol, 0.016 eq.), and potassium carbonate (0.666 g, 4.7 mmol, 2.0 eq.) in tetrahydrofuran (4 ml) was stirred at 40° C. overnight. The reaction mixture was cooled to room temperature, diluted with MTBE (methyl t-butyl ether) and poured into water. The aqueous phase was extracted three time with MTBE. The organic phases were combined, washed with aq. saturated NaCl solution, dried over MgSO₄, leading after solvent evaporation under reduced pressure to a crude mixture (1.27 g as a yellow oil).

Step b): In a 25 ml two-neck flask equipped with a magnetic stirrer, a reflux condenser and a thermometer, a mixture of the crude product obtained in the previous step (1.27 g), 32% aq. NaOH (0.98 ml, 7.85 mmol), and water (0.98 ml) was heated at reflux for 1 h. The reaction mixture was cooled to room temperature, extracted three times with MTBE and the combined organic phases were washed with aq. saturated NH₄Cl solution and with aq. saturated NaCl solution, dried over MgSO₄, leading after solvent evaporation under reduced pressure to a crude mixture (1.0 g as an orange oil; GC: 41% beta farnesene, 45% products isomers (E,E-Formula (IX), R′=Me)/E,Z-Formula (IX), R′=Me/E,Z-Formula (VIII), R′=Me/E,E-Formula (VIII), R′=Me); ¹³C-NMR: 10:31 (5E,9E)-6-(hydroxymethyl)-10,14-dimethylpentadeca-5,9,13-trien-2-one/(5Z,9E)-6-(hydroxymethyl)-10, 14-dimethylpentadeca-5,9,13-trien-2-one and 38:13 (5Z,8E)-5-(2-hydroxyethylidene)-9, 13-dimethyltetradeca-8, 12-dien-2-one/(5E,8E)-5-(2-hydroxyethylidene)-9,13-dimethyltetradeca-8, 12- dien-2-one) that was purified by Kugelrohr distillation yielding at 100° C. (0.03 mbar) a distilled fraction of beta farnesene (0.21 g, 83% purity, 62% yield) and at 180° C. (0.03 mbar) a distilled fraction of isomers ((E,E-Formula (IX), R′=Me)/E,Z-Formula (IX), R′=Me/E,Z-Formula (VIII), R′=Me/E,E-Formula (VIII), R′=Me; 0.25 g, 88% purity, 40% yield)

### Example 16: (5Z.9E)-6-(hydroxymethyl)-10, 14-dimethylpentadeca-5,9,13-trien-2-one (E,Z-Formula (IX), R′=Me), (5E,9E)-6-(hydroxymethyl)-10,14-dimethylpentadeca-5,9,13-trien-2-one (E,E-Formula (IX), R′=Me), (5Z,8E)-5-(2-hydroxyethylidene)-9,13-dimethyltetradeca-8,12-dien-2-one (E,Z-Formula (VIII), R′=Me) and (5E,8E)-5-(2-hydroxyethylidene)-9,13-dimethyltetradeca-8,12-dien-2-one (E,E-Formula (VIII), R′=Me)

In a 100 ml three-neck flask equipped with a magnetic stirrer, a reflux condenser and a thermometer, a mixture of beta farnesene/(E)-2-(4,8-dimethylnona-3,7-dien-1-yl)-2-vinyloxirane (E-Formula (II))/(E)-2-(6,10-dimethylundeca-1,5,9-trien-2-yl)oxirane (E-Formula (I)) (10 g; 29% beta farnesene, 32% E-Formula (II), 22% E-Formula (I); 25 mmol epoxides), methyl acetoacetate (Formula (VII), R=Me, R′=Me; 5.87 g, 50 mmol, 2.0 eq.), palladium (II) bis(acetylacetonate) (10.4 mg, 0.05 mmol, 0.002 eq.), PPh₃ (53 mg, 0.20 mmol, 0.008 eq.), and potassium carbonate (7.05 g, 50 mmol, 2.0 eq.) in tetrahydrofuran (40 ml) was stirred at 40° C. for 6 h. The reaction mixture was then treated with water (15 ml), cooled to 5° C., treated dropwise with 32% aq. NaOH (12.5 ml, 100 mmol, 2 eq.) and heated at reflux (66° C.) for 1 h. The resulting mixture was cooled to room temperature and diluted with MTBE (methyl t-butyl ether) and water. The aq. phase was extracted three times with MTBE and the combined organic phases were washed with aq. saturated NH₄Cl solution and with aq. saturated NaCl solution, dried over MgSO₄, leading after solvent evaporation under reduced pressure to a crude mixture (11.3 g as an orange oil; GC: 39% beta farnesene, 48% products isomers (E,E-Formula (IX), R′=Me)/E,Z-Formula (IX), R′=Me/E,Z-Formula (VIII), R′=Me/E,E-Formula (VIII), R′=Me); ¹³C-NMR: 14:40 (5E,9E)-6-(hydroxymethyl)-10,14-dimethylpentadeca-5,9, 13-trien-2-one/(5Z,9E)-6-(hydroxymethyl)-10,14- dimethylpentadeca-5,9,13-trien-2-one and 33:14 (5Z,8E)-5-(2-hydroxyethylidene)-9,13-dimethyltetradeca-8, 12-dien-2-one/(5E,8E)-5-(2-hydroxyethylidene)-9,13-dimethyltetradeca-8, 12- dien-2-one that was purified by Kugelrohr distillation yielding at 100° C. (0.03 mbar) a distilled fraction of beta farnesene (2.0 g, 90% purity, 63% yield) and at 180° C. (0.02 mbar) a distilled fraction of isomers (E,E-Formula (IX), R′=Me)/E,Z-Formula (IX), R′=Me/E,Z-Formula (VIII), R′=Me/E,E-Formula (VIII), R′=Me; 5.0 g, 92% purity, 66% yield).

### Example 17: (5Z,9E)-6-(hydroxymethyl)-10, 14-dimethylpentadeca-5,9,13-trien-2-one:

A solution of (E)-2-(4,8-dimethylnona-3,7-dien-1-yl)-2-vinyloxirane (E-Formula (II), 150 mg, 0.68 mmol) in acetone (20 ml) was treated at r.t. with Pd(PPh₃)₄ (31.5 mg, 0.027 mmol, 0.04 eq.) and heated at 50° C. for 24 h. The resulting mixture was cooled to r.t., treated with aq. saturated NaCl solution and the organic phase was separated and dried over Na₂SO₄, filtered through a pad of SiO₂ leading after solvent evaporation under reduced pressure to a crude mixture that was purified by flash chromatography (heptane/EtOAc 88:12 to 0:100) on SiO₂ yielding (5Z,9E)-6-(hydroxymethyl)-10,14-dimethylpentadeca-5,9,13-trien-2-one (E,Z-Formula (IX), R′=Me, 9.8 mg, 5% yield) as an oil.

Rf 0.28 (heptane/AcOEt 1:1)

### Example 18: (2E,5E)-6,10-dimethyl-2-(pent-4-en-1-ylidene)undeca-5,9-dien-1-ol (E,E-Formula (X)) and (2Z.5E)-6,10-dimethyl-2-(pent-4-en-1-ylidene)undeca-5,9-dien-1-ol (E,Z-Formula (X))

At −30 ° C., a stirred solution of the (E)-2-(4,8-dimethylnona-3,7-dien-1-yl)-2-vinyloxirane (E-Formula (II), 200 mg, 0.908 mmol) in THF (0.6 ml) was treated with copper bromide dimethyl sulfide complex (4.85 mg, 0.024 mmol, 0.026 eq.) followed by the dropwise addition (1 h) of a solution of 2M allyl magnesium chloride in THF (0.681 ml, 1.361 mmol, 1.5 eq.). After stirring at −30° C. for 2 h, the reaction mixture was warmed to −20° C., treated with aq. saturated NH₄Cl and brine solution. The organic phase was separated, dried (Na₂SO₄), and concentrated leading to a residue (295 mg) that was purified by flash chromatography (heptane/AcOEt 95:5 to 60.40) to afford a 85:15 mixture of (2E,5E)-6, 10-dimethyl-2-(pent-4-en-1-ylidene)undeca-5,9-dien-1-ol and (2Z,5E)-6,10-dimethyl-2-(pent-4-en-1-ylidene)undeca-5,9-dien-1-ol (165 mg, 69% yield, colorless oil).

¹³C NMR (100 MHZ, CDCl₃, major isomer (2E,5E)) δ ppm 139.17 (s, 1 C), 138.20 (d, 1 C), 135.38 (s, 1 C), 131.19 (s, 1 C), 126.06 (d, 1 C), 124.20 (d, 1 C), 123.79 (d, 1 C), 114.61 (t, 1 C), 66.90 (t, 1 C), 39.61 (t, 1 C), 33.75 (t, 1 C), 28.07 (t, 1 C), 26.86 (t, 2 C), 26.57 (t, 1 C), 25.57 (q, 1 C), 17.56 (q, 1 C), 15.87 (q, 1 C).

¹³C NMR (100 MHZ, CDCl₃, minor isomer (2Z,5E)) δ ppm 138.65 (s, 1 C), 138.08 (d, 1 C), 135.20 (s, 1 C), 131.15 (s, 1 C), 127.73 (d, 1 C), 124.23 (d, 1 C), 123.90 (d, 1 C), 114.86 (t, 1 C), 59.97 (t, 1 C), 39.61 (t, 1 C), 34.99 (t, 1 C) 33.95 (t, 1 C), 26.92 (t, 1 C), 26.74 (t, 1 C), 26.63 (t, 1 C), 25.57 (q, 1 C), 17.56 (q, 1 C), 15.95 (q, 1 C).

### Data of the Isomer Mixture:

¹H NMR (400 MHZ, CDCl₃) 0 ppm 5.88-5.73 (m, 1H), 5.42 (br. t, J=6.8, 0.85 H), 5.30 (br. t, J=7.2, 0.15 H), 5.17-4.93 (m, 4H), 4.10 (s, 2×0.15 H), 4.02 (s, 2×0.85 H), 2.21-1.93 (m, 12H), 1.93-1.80 (br. s, 1H, OH), 1.67 (br. s, 3H), 1.59 (s, 6H).

GC-MS (EI): 262 (1), 244 (1), 229 (1), 219 (1), 201 (2), 173 (2), 162 (4), 147 (3), 137 (7), 133 (7), 121 (15), 107 (9), 105 (9), 95 (18), 93 (16), 91 (16), 81 (34), 79 (17), 69 (100), 67 (21), 59 (1), 55 (19), 43 (8), 41 (62), 31 (2).

### Example 19: (5E,9E)-6-(hydroxymethyl)-10,14-dimethylpentadeca-5,9,13-trien-2-one (E,E-Formula (IX), R′=Me) and (5Z,9E)-6-(hydroxymethyl)-10,14-dimethylpentadeca-5,9,13-trien-2-one (E,Z-Formula (IX), R′=Me)

A solution of CuCl (64.7 mg, 0.65 mmol, 1 eq.) and PdCl₂ (19.3 mg, 0.109 mmol, 0.2 eq.) in 6:1 DMF/H₂O (7 mL) was stirred under O₂ atmosphere at r.t. for 2 h. The 85:15 mixture of (2E,5E)-6, 10-dimethyl-2-(pent-4-en-1-ylidene)undeca-5,9-dien-1-ol (E,E-Formula (X)) and (2Z,5E)-6,10-dimethyl-2-(pent-4-en-1-ylidene)undeca-5,9-dien-1-ol (E,Z-Formula (X)) obtained from Example 17 (165 mg, 0.63 mmol) was added and the resulting solution was stirred overnight, diluted with ether and treated with aq. saturated NH₄Cl solution. The organic layer was washed three times with water and with brine, dried (Na₂SO₄), and concentrated leading to 160 mg of crude product that was purified by flash chromatography (heptane/AcOEt) to afford a 85:15 mixture of (5E,9E)-6-(hydroxymethyl)-10, 14-dimethylpentadeca-5,9,13-trien-2-one and (5Z,9E)-6-(hydroxymethyl)-10, 14-dimethylpentadeca-5,9,13-trien-2-one (75 mg, 43% yield, colorless oil).

While the invention has been described with reference to certain examples, those skilled in the art will appreciate that various modifications, changes, omissions, and substitutions can be made without departing from the spirit of the disclosure. It is intended, therefore, that the invention be limited by the scope of the following claims and their equivalents. Unless otherwise stated, the features of any dependent claim can be combined with the features of any of the other dependent claims and any of the independent claims.

1. A method for making a compound of Formula (I), a compound of Formula (II) or a mixture thereof, 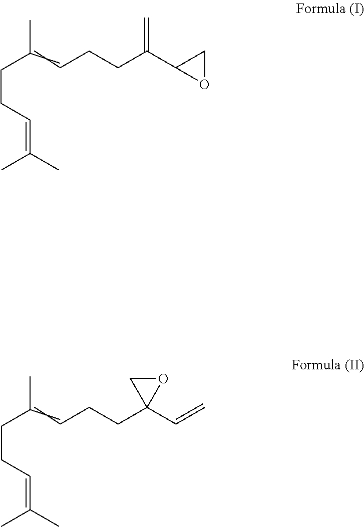 [SMILES: C=C(CCC=C(C)CCC=C(C)C)C1CO1 ; C=CC1(CCC=C(C)CCC=C(C)C)CO1]
wherein the method comprises contacting a compound of Formula (III) with a catalyst comprising a metal complexed with a salen ligand or a porphyrin ligand, wherein the salen ligand has Formula (IV) and wherein the porphyrin ligand has Formula (XI), 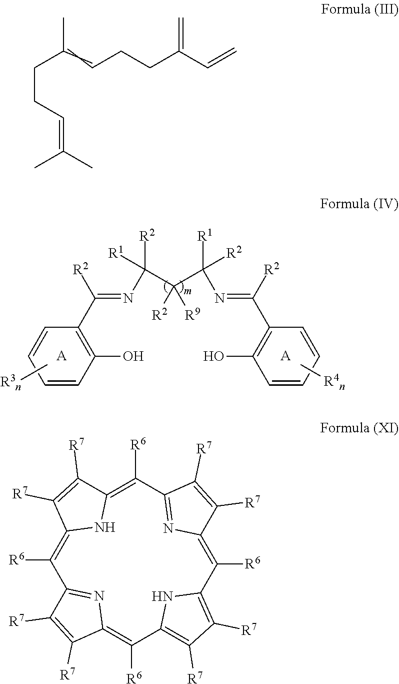
wherein
each ring A is independently selected from phenyl or naphthyl;
each R¹ is independently hydrogen, an alkyl group, an aryl group, or both R¹ together with the carbon atoms to which R¹ are attached form a cycloalkylene group, an arylene group, two or more cycloalkylene groups connected by a single bond or an alkyl group, two or more arylene groups connected by a single bond or an alkyl group, or a cycloalkylene and an arylene group connected by a single bond or an alkyl group;
each R² is independently hydrogen or an alkyl group;
R³ is independently an alkyl group, an alkoxy group, an alkaryl group, an alkylsilyl ether group, or a halide;
R⁴ is independently an alkyl group, an alkoxy group, an alkaryl group, an alkylsilyl ether group, or a halide;
R⁹ is selected from hydrogen, an optionally substituted alkyl group, an optionally substituted aryl group, hydroxyl or an amino group;
each n is selected from 0 to 4;
m is selected from 0 to 4;
each R⁶ is independently selected from hydrogen, an optionally substituted alkyl group, an optionally substituted alkylidene group, an optionally substituted aryl group, or an optionally substituted heteroaromatic group; and
each R⁷ is independently selected from hydrogen, an alkyl group, an ester-containing alkyl group or a carboxylic acid containing alkyl group.

2. A method for making a compound of Formula (V), a compound of Formula (VI) or a mixture thereof, 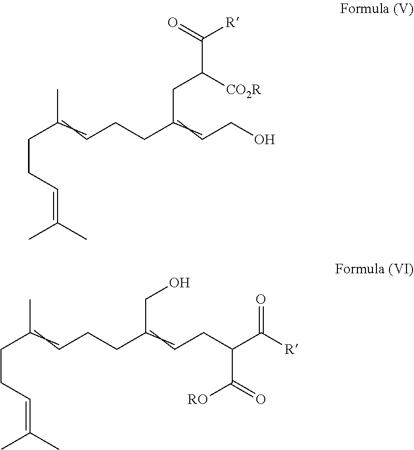
wherein the method comprises contacting a compound of Formula (I), a compound of Formula (II) or a mixture thereof with a compound of Formula (VII) and a palladium catalyst 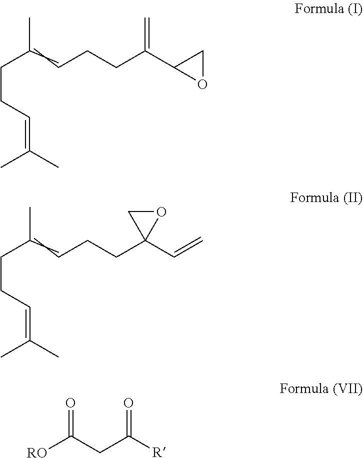
wherein R is selected from the group consisting of hydrogen and a C1 to C6 alkyl group; and R′ is selected from the group consisting of a C1 to C6 alkyl group and an OR group.

3. The method of claim 2, wherein the method produces a mixture of E,E- and E,Z-Formula (V) and/or a mixture of E,E- and E,Z-Formula (VI)

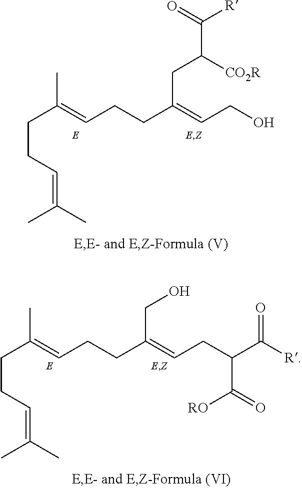

4. A method for making a compound of Formula (VIII), a compound of Formula (IX) or a mixture thereof, 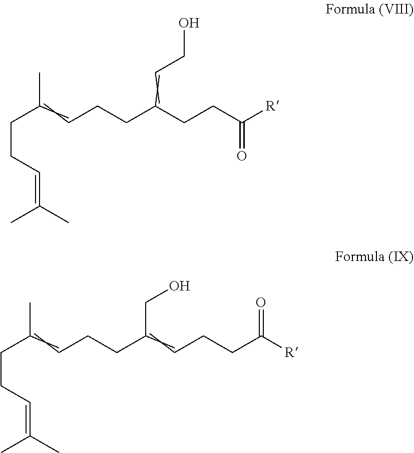
wherein the method comprises hydrolysis and decarboxylation of a compound of Formula (V), a compound of Formula (VI), or a mixture thereof, 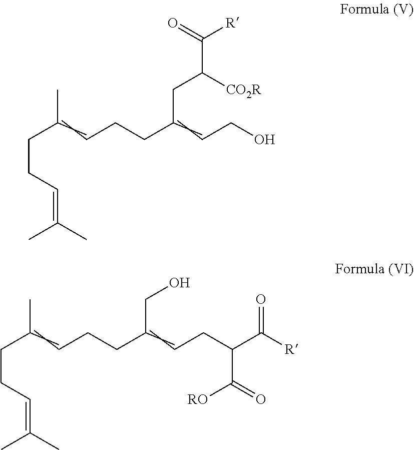
wherein R′ is selected from the group consisting of a C1 to C6 alkyl group and an OR group, and wherein R is selected from the group consisting of hydrogen or a C1 to C6 alkyl group.

5. The method of claim 4, wherein hydrolysis and decarboxylation of a compound of Formula (V), a compound of Formula (VI), or a mixture thereof comprises contacting the compound of Formula (V), the compound of Formula (VI), or the mixture thereof with an acid, a base, optionally in the presence of water, or a nucleophile.

6. A method for making a compound of Formula (VIII), a compound of Formula (IX) or a mixture thereof, wherein R′ is methyl, 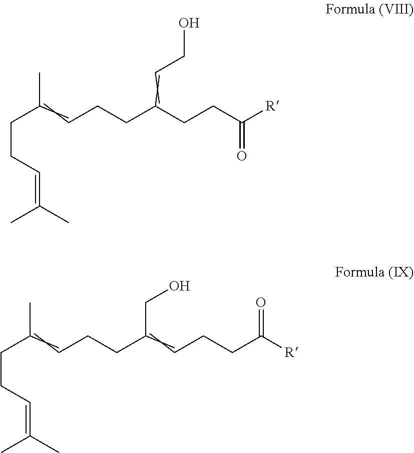
wherein the method comprises contacting a compound of Formula (I), a compound of Formula (II) or a mixture thereof with acetone and a palladium catalyst.

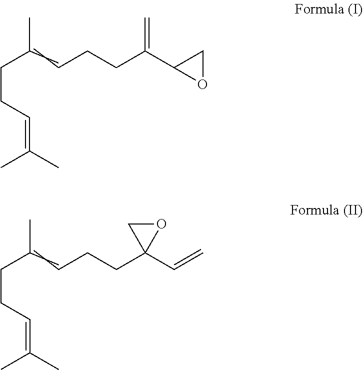 [SMILES: C=C(CCC=C(C)CCC=C(C)C)C1CO1 ; C=CC1(CCC=C(C)CCC=C(C)C)CO1]

7. A method for making a compound of Formula (IX), a compound of Formula (VIII), or a mixture thereof, wherein R′ is methyl, 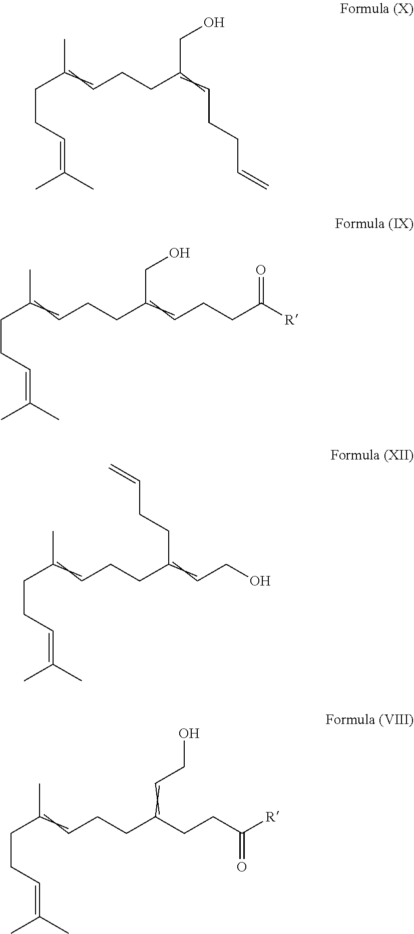
the method comprising a palladium-catalysed Wacker oxidation of a compound of Formula (X), a compound of Formula (XII), or a mixture thereof.

8. A method for making a compound of Formula (X), a compound of Formula (XII), or a mixture thereof, 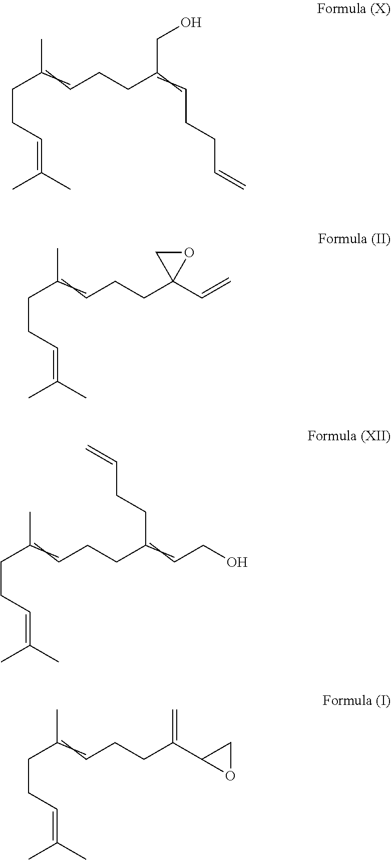
wherein the method comprises a copper-catalysed alkylation of a compound of Formula (II), a compound of Formula (I), or a mixture thereof

9. (canceled)

10. A method of separation of a mixture of a compound of Formula (I) and a compound of Formula (II), 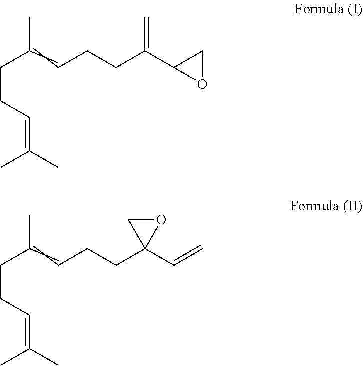 [SMILES: C=C(CCC=C(C)CCC=C(C)C)C1CO1 ; C=CC1(CCC=C(C)CCC=C(C)C)CO1]
wherein the method comprises the distillation of a mixture of a compound of Formula (I) and a compound of Formula (II).

11. A compound of Formula (V), a compound of Formula (VI) or a mixture thereof, 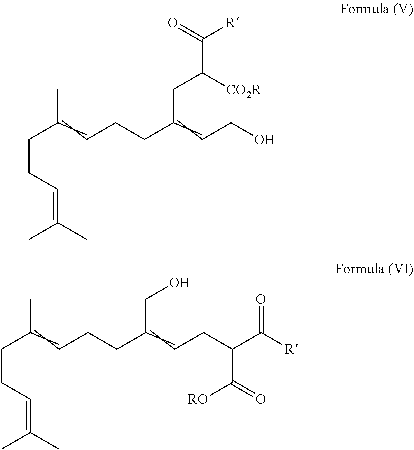
wherein R is selected from the group consisting of hydrogen and a C1 to C6 alkyl group; and R′ is selected from the group consisting of a C1 to C6 alkyl group and an OR group.

12. A compound of Formula (X), a compound of Formula (XII), or a mixture thereof

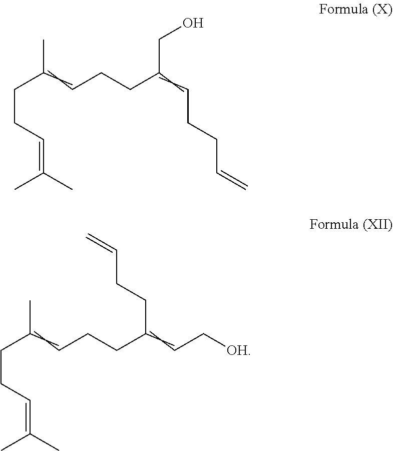

13. (canceled)

14. (canceled)

15. (canceled)

16. The method of claim 1, further comprising contacting the compound of Formula (I), the compound of Formula (II), or the mixture thereof with a compound of Formula (VII) and a palladium catalyst to obtain a compound of Formula (V), a compound of Formula (VI), or a mixture thereof

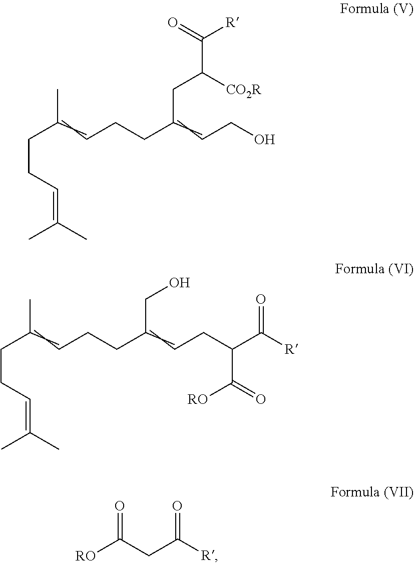

17. The method of claim 16, further comprising optionally followed by hydrolysis and decarboxylation of the compound of Formula (V), the compound of Formula (VI), or a mixture thereof to obtain a compound of Formula (VIII), a compound of Formula (IX), or a mixture thereof 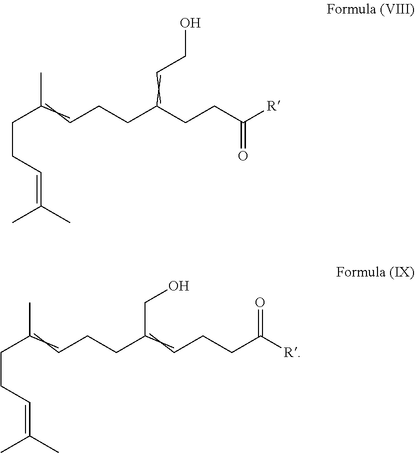
wherein R is selected from the group consisting of hydrogen and a C1 to C6 alkyl group;
and R′ is selected from the group consisting of a C1 to C6 alkyl group and an OR group.

18. A compound of Formula (VIII), a compound of Formula (IX) or a mixture thereof produced by the method of claim 5.

19. A compound of Formula (VIII), a compound of Formula (IX) or a mixture thereof produced by the method of claim 6.
